(19) United States
(12) Patent Application Publication
(10) Pub. No.: US 2024/0287454 A1
(43) Pub. Date: Aug. 29, 2024

(54) SINGLE VESSEL EXPANSION OF LYMPHOCYTES

(71) Applicants: TIGEN PHARMA SA, Lausanne (CH); LUDWIG INSTITUTE FOR CANCER RESEARCH LTD, Zürich (CH); CENTRE HOSPITALIER UNIVERSITAIRE VAUDOIS (CHUV), Lausanne (CH)

(72) Inventors: George Coukos, Lausanne (CH); Alexandre Harari, Lausanne (CH); Simone Steiner, Lausanne (CH); Florence Salmon, Lausanne (CH)

(73) Assignees: TIGEN PHARMA SA, Lausanne (CH); LUDWIG INSTITUTE FOR CANCER RESEARCH LTD, Zürich (CH); CENTRE HOSPITALIER UNIVERSITAIRE VAUDOIS (CHUV), Lausanne (CH)

(21) Appl. No.: 18/288,893

(22) PCT Filed: Apr. 29, 2022
(86) PCT No.: PCT/EP2022/061639

§ 371 Date: Oct. 30, 2023

(30) Foreign Application Priority Data
Apr. 30, 2021 (EP) 21171565.1

(51) Int. Cl. C12N 5/0783; A61K 35/17

(52) U.S. Cl. CPC C12N 5/0636; A61K 35/17; C12N 2501/2302; C12N 2502/1107

(57) ABSTRACT

The present invention relates to a population of lymphocytes comprising at least 90% CD3+ T cells and less than 5% B cells, wherein at least 70% of said T cell portion are viable, at least 20% are CD27/CD28 double positive and less than 10% are triple positive for CD45RA, CD57 and KLRG1 and a method for expansion of a population of lymphocytes specific for one or more antigens comprising a single culture phase.

### 1. BACKGROUND

The present invention relates to lymphocytes for use in targeted tumor immunotherapies such as adoptive T cell therapy, as well as methods of production and kits comprising such cells. The lymphocytes are preferably human lymphocytes such as NK cells or T cells, including CD3+ T cells, CD8+ T cells, CD4+ T cells, and γδ T cells. Most preferably, the cells of the invention are primary human T cells. The invention provides a population of fit lymphocytes exhibiting a specific marker profile (i.e. high CD27/CD28 expression and low CD45RA/CD57/KLRG1 expression) and specificity for one or more defined antigens. Such antigens can be antigens characteristic of disease state, including infectious disease (such as viral or bacterial infections) and cancers, and/or may be neoantigens selected from known neoantigens or identified in samples obtained from the subject, e.g. patient to be treated. Provided are also pharmaceutical compositions comprising such lymphocytes, in particular, for use in a method of treatment of diseases characterized by the antigen or neoantigen expression.

The use of adoptive cell therapy (ACT), e.g. T cell therapy, has been demonstrated as an effective treatment for multiple diseases, including cancers. Adoptive cell therapy is a powerful treatment approach using naturally occurring antigen-specific lymphocytes, e.g. T cells, or lymphocytes rendered antigen-specific by genetic engineering, e.g. to express recombinant T cell receptors or chimeric antigen receptors. However, a particular issue facing the more widespread development and use of such therapies has been the complexity and costs associated with development and selection of the cell therapeutic, i.e. the selection and expansion of cells having desired specificity in the quantities and quality required.

A common drawback of adoptive cell therapy is that reaching sufficient cell numbers (approximately 109 cells) usually requires expanding cells ex vivo for several weeks and/or involves the use of multiple culture phases wherein the cells are typically frozen between phases. As a consequence, a large fraction of the cells may be lost to the effects of freeze-thawing; additionally prolonged culturing can cause T cells to become terminal effector cells that may die shortly after infusion to the patient before reaching a target cell, tissue and/or organ. Accordingly, there is a need in the art for shorter expansion protocols that avoid freeze-thawing cycles and result in younger and fitter lymphocyte populations, i.e. populations of antigen specific T cells that are not terminally differentiated and comprise a low fraction of terminal effectors.

### 2. SUMMARY

The present invention relates to an improved method for expanding lymphocytes, in particular antigen-specific lymphocytes, ex vivo. The methods of the invention bears the advantage that high cell numbers (e.g. at least approximately 10 cells) can be achieved from a patient sample in a controlled single culture vessel without the need to transfer the cell culture to a larger culture vessel during the process. Furthermore, the methods of the invention provide a more rapid expansion of the cells relative to available methods. As a consequence, younger cell populations, characterized by a small fraction of terminal effector cells and, preferably, high stemness can be obtained. Those characteristics allow younger cells to efficiently proliferate after re-infusion, reaching target cells, tissue(s) or organ(s) before differentiating into terminal effector cells. While the terminal effector cells are involved in the immediate attack of cancer cells, the younger cells are expected to provide a durable response.

The invention relates to the following items:
A1. A population of lymphocytes comprising at least 90% CD3+ T cells and less than 5% B cells, wherein at least 70% of said T cell portion are viable, at least 50% are CD27/CD28 double positive and less than 10% are triple positive for CD45RA, CD57 and KLRG1.
A2. The population of lymphocytes according to item A1, wherein said T cells are specific for one or more markers and are not positive for KLRG1 as determined by flow cytometry.
A3. The population of lymphocytes according to any one of items A1 or A2, wherein said T cell portion has an average telomere length of at least 5 kb, 6 kb, 7 kb, 8 kb, 9 kb.
A4. The population of lymphocytes according to any one of items A1 to A3, wherein less than 10% of said T cell portion secrete at least one protein from the group consisting of: IFN-γ, TNF-α, IL-4, IL-5, Granzyme B and Perforin.
A5. The population of lymphocytes according to any one of items A1 to A4, wherein at least 50%, at least 60%, at least 70%, at least 80% or at least 90% of the T cells in said T cell portion are CD8+ T cells.
A6. The population of lymphocytes according to any one of items A1 to A5, wherein at least two T cells in said T cell portion are directed against different antigens.
A7. A method for expansion of a population of lymphocytes specific for one or more antigens comprising a single culture phase, wherein said single phase comprises
   a) culturing a tissue or blood sample from a subject in the presence of said one or more antigens, which sample is known or suspected to contain lymphocytes; or
   b) culturing T cells in the presence of said one or more antigens, which T cells are isolated from a tissue or blood sample from a subject;
   wherein said culturing is continued until said T cell population is at least 10×10⁸ cells, and wherein said culturing is at temperatures greater than 0° C. during said single culture phase.
A8. The method according to item A7(a) or A7(b) wherein said sample or said T cells are maintained at temperatures greater than 0° C. subsequent to isolation from said subject and prior to said culture.
A9. The method according to item A7 or A8, wherein said antigens are isolated or determined from said subject or said sample.
A10. The method according to any one of items A7 to A9, wherein said one or more antigens are soluble peptides including in the culture medium at concentrations of 0.1 to 10 μg/m.
A11. The method according to any one of items A7 to A10, wherein said culturing comprises co-culture with antigen-presenting cells (APCs) engineered to present said one or more antigens.
A12. The method according to item All, wherein said APCs comprise B cells.
A13. The method according to item A12, wherein said B cells are recombinantly engineered to express said one or more antigens.
A14. A population of T cells obtainable by the method of any one of items A7 to A13.
A15. The population of lymphocytes according to any one of items A1 to A6, or the population of T cells according to item A14 for use as a medicament.
B1. A method for expansion of a population of lymphocytes specific for one or more antigens in a controlled single culture vessel, the method comprising the steps of:
   a) culturing a tissue or blood sample from a subject in the presence of said one or more antigens, which sample is known or suspected to contain lymphocytes; or
   b) culturing lymphocytes in the presence of said one or more antigens, which lymphocytes are isolated from a tissue or blood sample from a subject;
   wherein the lymphocytes are cultured in a conditioned culture medium.
B2. The method according to item B1, wherein the conditioned culture medium is a culture medium in which at least one of the following parameters is monitored and adjusted if necessary: pH, dissolved oxygen (DO) concentration, glucose concentration, lactate concentration and/or temperature.
B3. The method according to item B2, wherein the conditioned culture medium is a culture medium in which pH, dissolved oxygen (DO) concentration, glucose concentration, lactate concentration and temperature are monitored and adjusted if necessary.
B4. The method according to any one of items B1 to B3, the method comprising a step of adjusting he culture volume to the expansion rate of the lymphocytes.
B5. The method according to item B4, wherein the culture volume increases at least by a factor of 2, 3, 4, 5 or 6 during expansion of the lymphocytes.
B6. The method according to any one of items B1 to B5, the method comprising a step of dynamic culturing the lymphocyte culture with the conditioned culture medium.
B7. The method according to any one of items B1 to B6, wherein the tissue sample is derived from a tumor, in particular wherein the tissue sample is a tumor sample.
B8. The method according to item B7, wherein the tumor comprises at least one neoantigen.
B9. The method according to any one of items B1 to B8, wherein the lymphocytes comprise tumor-infiltrating lymphocytes, in particular wherein the tumor-infiltrating lymphocytes are T cells.
B10. The method according to any one of items B1 to B9, wherein one or more antigens are added to the culture medium in the form of peptides.
B11. The method according to item B10, wherein the peptides are added to the culture medium at a concentration of 0.1 to 10 μg/ml.
B12. The method according to any one of item B1 to B11, wherein said culturing step comprises a step of co-culturing the lymphocytes with antigen-presenting cells (APCs).
B13. The method according to item B12, wherein the antigen-presenting cells (APCs) are engineered to present one or more antigens.
B14. The method according to item B12 or B13, wherein the antigen-presenting cells (APCs) comprise or are B cells.
B15. The method according to item B14, wherein the B cells are obtained by apheresis.
B16. The method according to item B14 or B15, wherein the B cells are activated before addition of the lymphocytes.
B17. The method according to item B16, wherein the B cells are activated with IL-21, IL-4 and/or CD40L.
B18. The method according to any one of items B12 to B17, wherein the antigen-presenting cells (APCs) are genetically engineered to express one or more transgene.
B19. The method according to item B18, wherein the genetically engineered APCs have been obtained by transfecting the APCs with a nucleic acid encoding the one or more transgene.
B20. The method according to item B18 or B19, wherein at least one of the one or more transgenes encodes an immunomodulator.
B21. The method according to item B20, wherein the immunomodulator is selected from the group consisting of: OX40L, 4-1BBL, CD80, CD86, CD83, CD70, CD40L, GITR-L, CD127L, CD30L (CD153), LIGHT, BTLA, ICOS-L (CD275), SLAM (CD150), CD662L, interleukin-12, interleukin-7, interleukin-15, interleukin-17, interleukin-21, interleukin-4, Bc16, Bcl-XL, BCL-2, MCL1, STAT-5, and activators of one or more signaling pathways (e.g. the JAK/STAT pathway, the Akt/PKB signaling pathway, the BCR signaling pathway, and/or the BAFF/BAFFR signaling pathway).
B22. The method according to item B20 or B21, wherein the immunomodulator is one or more of OX40L, 4-1BBL and/or interleukin 12.
B23. The method according to any one of items B7 to B22, wherein the presence of at least one of the one or more antigens has been confirmed in the tumor sample comprising the lymphocytes prior to the culturing step.
B24. The method according to any one of items B7 to B23, wherein at least one of the one or more antigens is a neoantigen and wherein the presence of said neoantigen has been confirmed in the tumor sample comprising the lymphocytes prior to the culturing step.
B25. The method according to item B23 or B24, wherein confirming the presence of at least one of the one or more antigens in the tumor sample comprises a step of sequencing genomic DNA that has been obtained from the tumor sample.
B26. The method according to any one of items B1 to B25, wherein the method comprises a step of activating the lymphocytes during culturing.
B27. The method according to item B26, wherein the activation step comprises the addition of a CD3 agonist to the culture medium.
B28. The method according to item B27, wherein the CD3 agonist is added to the culture medium 1, 2, 3, 4, 5, 6, 7, 8, 9, 10, 11 or 12 days after the beginning of the culturing.
B29. The method according to any one of items B21 to B28, wherein the culture medium is supplemented with human AB serum and/or IL-2.
B30. The method according to any one of items B1 to B29, wherein said culturing is continued until said T cell population reaches at least 10⁷ cells.
B31. The method according to any one of items B1 to B30 wherein said culturing is performed at temperatures of greater than 0° C. during said single culture phase.
B32. The method according to any one of items B1 to B31, wherein said sample or said lymphocytes are maintained at temperatures greater than 0° C. subsequent to isolation from said subject and prior to said culture.
B33. A population of lymphocytes obtainable by the method of any one of items B1 to B32.
B34. A population of lymphocytes comprising at least 90% CD3+ T cells and less than 5% B cells, wherein at least 70% of said T cell portion are viable, at least 20% are CD27/CD28 double positive and less than 10% are triple positive for CD45RA, CD57 and KLRG1.
B35. The population of lymphocytes according to item B34, wherein said T cells are specific for one or more antigens.
B36. The population of lymphocytes according to any one of items B34 to B36, wherein less than 15% of said T cell portion secrete at least one protein from the group consisting of: TNF-α, IL-4 and IL-5.
B37. The population of lymphocytes according to any one of items B34 to B36, wherein at least 50%, at least 60%, at least 70%, at least 80% or at least 90% of the T cells in said T cell portion are CD8⁺ T cells.
B38. The population of lymphocytes according to any one of items B34 to B37, wherein at least two T cells in said T cell portion are directed against different antigens.
B39. The population of lymphocytes according to item B38, wherein at least one antigen is a neoantigen.
B40. The population of lymphocytes according to any one of items B34 to B39, wherein said T cell portion comprises at least 10⁷ T cells.
B41. A pharmaceutical composition comprising the population of lymphocytes according to any one of items B33 to B40.
B42. The pharmaceutical composition according to item B41, wherein the lymphocytes are suspended in a pharmaceutically acceptable buffer.
B43. The pharmaceutical composition according to item B42, wherein the pharmaceutically acceptable buffer comprises about 0.9% NaCl and, optionally, up to 15% DMSO.
B44. The population of lymphocytes according to any one of items B33 to B40 or the pharmaceutical composition according to any one of items B41 to B43 for use as a medicament.
B45. The population of lymphocytes according to any one of items B33 to B40 or the pharmaceutical composition according to any one of items B41 to B43 for use in cancer therapy.
B46. The population of lymphocytes or the pharmaceutical composition for use according to item B45, wherein the cancer therapy is adoptive cell therapy.
B47. The population of lymphocytes or the pharmaceutical composition for use according to item B45 or B46, wherein the cancer therapy is autologous cell therapy.
B48. A method for treating cancer, the method comprising the steps of:
   a) providing a population of lymphocytes according to any one of items B33 to B40 or a pharmaceutical composition according to any one of items B41 to B43; and
   b) infusing the population of lymphocytes or the pharmaceutical composition into a subject suffering from cancer.
B49. A method for treating cancer in a subject, the method comprising the steps of:
   a) surgically removing a tumor from a subject or taking a biopsy from a subject's tumor;
   b) identifying at least one tumor antigen in the tumor sample obtained in step (a);
   c) expanding lymphocytes comprised in the tumor sample obtained in step (a) with the method according to any one of items B1 to B33, wherein the lymphocytes are expanded in the presence of at least one tumor antigen that has been identified in step (b) to be present in the tumor sample;
   d) infusing the expanded lymphocytes obtained in step (c) into the subject from which the tumor sample has been obtained.
B50. The method according to item B49, wherein the tumor antigen is a tumor-associated antigen or a tumor-specific antigen.
B51. The method according to any one of items B48 to B49, wherein the lymphocytes comprise tumor-infiltrating lymphocytes (TILs).
B52. The method according to item B51, wherein the TILs specifically recognize one or more tumor antigens.
B53. The method according to item B52, wherein at least one tumor antigen is a neoantigen.

Accordingly, in a particular embodiment, the invention relates to a population of lymphocytes comprising at least 90% CD3+ T cells and less than 5% B cells, wherein at least 70% of said T cell portion are viable and wherein at least 50% of said T cell portion are CD27 and/or CD28 positive.

That is, in certain embodiments, the invention relates to a population of lymphocytes comprising at least 90% CD3+ T cells. The term “CD3+ T cells”, as used herein, refers to a type of cells that express the CD3 marker. “CD3”, as used herein, refers to a cluster of differentiation 3, a protein complex composed of four distinct chains. In mammals, the complex contains a CD3y chain, a CD36 chain, and two CD3E chains. These chains associate with a molecule known as the T cell receptor (TCR) and the ζ-chain to generate an activation signal in T lymphocytes. The TCR, ζ-chain, and CD3 molecules together comprise the TCR complex.

In certain embodiments, at least 90%, at least 91%, at least 92%, at least 93%, at least 94%, at least 95%, at least 96%, at least 97%, at least 98% or at least 99% of the lymphocytes in the population of lymphocytes are CD3+ T cells.

The skilled person is aware of methods to determine the percentage of CD3+ T cells in a population of cells. For example, the percentage of CD3+ T cells in a population of cells may be determined by flow cytometry, using antibodies directed against CD3 and/or other suitable T cell-specific surface markers.

In certain embodiments, at least 90%, at least 91%, at least 92%, at least 93%, at least 94%, at least 95%, at least 96%, at least 97%, at least 98% or at least 99% of the lymphocytes in the population of lymphocytes are CD3+ and CD45+ T cells, as determined by flow cytometry.

In certain embodiments the population of lymphocytes may comprise up to 10% of impurities.

In certain embodiments, the population of lymphocytes is obtained by contacting a patient sample comprising lymphocytes or isolated lymphocytes with B cells, in particular antigen-presenting B cells. Thus, in certain embodiments, the population of lymphocytes may comprise a population of B cells. In certain embodiments, the population of lymphocytes comprises less than 5%, less than 4%, less than 3%, less than 2% or less than 1% B cells.

Alternatively, the population of lymphocytes may comprise between 0.1% and 5% B cells, between 0.1% and 4% B cells, between 0.1% and 3% B cells, between 0.1% and 2% B cells, or between 0.1% and 1% B cells.

Even if lymphocytes are initially cultured in the presence of B cells, it is to be understood that the final population of lymphocytes may be free of B cells. That is because B cells are usually not able to survive in T cell specific media for prolonged periods. Thus, in certain embodiments, the population of lymphocytes according is substantially free of B cells. That is, the number of B cells in the population may be below the limit of quantification by flow cytometry.

The skilled person is aware of methods to determine the percentage of B cells in a population of cells. For example, B cells may be identified by flow cytometry using antibodies against B cell specific surface markers, such as CD19 or CD20.

The term “B cell”, as used herein, refers to a type of lymphocyte that plays a major role in the humoral immune response, as opposed to the cell-mediated immune response, which is governed by T cells. B cells are characterized by the presence of a B cell receptor (BCR) on their outer surface which allows the B cell to bind to its specific antigen. The principal functions of a B cell are (i) to produce antibodies against the specific antigens which it recognizes, (ii) to perform the role of antigen-presenting cells (APCs) and (iii) to eventually develop into memory B cells after activation by interacting with its cognate antigen. B cells are an essential component of the adaptive immune system. The term “B cell” includes long-lived plasma cells and memory B cells. The term “long-lived plasma B cell”, as used herein, refers to a sub-type of B cells that reside primarily in the bone marrow and continuously secrete antibodies. The term “memory B cell”, as used herein, refers to a sub-type of B cells that are formed following a primary infection and activation by interacting with its cognate antigen, reside primarily in peripheral lymphoid tissues and, upon re-encounter with the priming antigen, differentiate into antibody-secreting cells (ASC) thus amplifying the antibody response. In certain embodiments, the B cell is a memory B cell.

Other impurities may be cells that were comprised in the sample from which the lymphocytes and/or the B cells originate. For example, in certain embodiments, the lymphocytes originate from tumor samples. In such embodiments, the preparation of lymphocytes may comprise a residual fraction of tumor cells. The abundance of tumor cells in the final population of lymphocytes can be determined by flow cytometry, for example by determining the abundance of CD45-negative cells in the population of lymphocytes. Alternatively or in addition, residual tumor cells in the population of lymphocytes may be detected by qPCR as known in the art.

In certain embodiments, the population of lymphocytes comprises less than 10%, less than 9%, less than 8%, less than 7%, less than 6%, less than 5%, less than 4%, less than 3% or less than 2% of B cells (CD19+ or CD20+) and/or tumor cells (CD45-).

Alternatively, the population of lymphocytes may comprise between 0.1% and 10%, between 0.1% and 9%, between 0.1% and 8, between 0.1% and 7%, between 0.1% and 6%, between 0.1% and 5%, between 0.1% and 4% B cells, between 0.1% and 3%, between 0.1% and 2%, or between 0.1% and 1% of B cells (CD19+ or CD20+) and/or tumor cells (CD45−).

The population of lymphocytes according to the invention may further comprise NK cells (CD3−, CD56+) and/or NKT cells (CD3+, CD56+). In certain embodiments, the population of lymphocytes may thus comprise between 0.1% and 10%, between 0.1% and 9%, between 0.1% and 8, between 0.1% and 7%, between 0.1% and 6%, between 0.1% and 5%, between 0.1% and 4% B cells, between 0.1% and 3%, between 0.1% and 2%, or between 0.1% and 1% of B cells (CD19+ or CD20+) and/or tumor cells (CD45−) and/or NK cells(CD3−, CD56+) and/or NKT cells (CD3+, CD56+).

Within the present invention, it is preferred that at least 70% of the CD3+ T cells in the population of cells are viable cells. Various methods to determine the viability of a T cell are known in the art and are commercially available. Without limitation, the viability of T cells in the population of lymphocytes may be determined in a proliferation assay or by live/dead staining.

In certain embodiments, at least 70%, at least 75%, at least 80%, at least 85%, at least 90% or at least 95% of the CD3+ T cells in the population of lymphocytes are viable CD3+ T cells.

In certain embodiments, the viability of the CD3+ T cells in the population of lymphocytes can be determined by flow cytometry using the cell surface marker Annexin V (AnnV) and the nucleic acid dye 7-Amino-Actinomycin D (7AAD) or propidium iodide (PI). Viable cells are double negative for AnnV and 7AAD/PI. Early apoptotic cells are positive for AnnV and negative for 7AAD/PI. Late apoptotic cells are positive for AnnV and positive for 7AAD/PI. Dead cells are negative for AnnV and positive for 7AAD/PI.

In certain embodiments, at least 70%, at least 75%, at least 80%, at least 85%, at least 90% or at least 95% of the CD3+ T cells in the population of lymphocytes are double negative for AnnV and 7AAD/PI.

Annexin V (or annexin V) is a cellular protein in the annexin group. In flow cytometry, annexin V is commonly used to detect apoptotic cells by its ability to bind to phosphatidylserine, a marker of apoptosis when it is on the outer leaflet of the plasma membrane. 7-Aminoactinomycin D (7-AAD) is a fluorescent chemical compound with a strong affinity for DNA. It is used as a fluorescent marker for DNA in fluorescence microscopy and flow cytometry. It is taken up by cells when the cell membrane integrity is lost and intercalates in double-stranded DNA, with a high affinity for GC-rich regions, making it useful for chromosome banding studies.

Propidium iodide (or PI) is a fluorescent intercalating agent that can be used to stain cells and nucleic acids. PI binds to DNA by intercalating between the bases with little or no sequence preference. Propidium iodide is used as a DNA stain in flow cytometry to evaluate cell viability or DNA content in cell cycle analysis, or in microscopy to visualize the nucleus and other DNA-containing organelles. Propidium Iodide is not membrane-permeable, making it useful to differentiate necrotic, apoptotic and healthy cells based on membrane integrity.

Viability may further be determined by using a cell counter such as, without limitation, a NucleoCounter NC-202. That is, in certain embodiments, at least 70%, at least 75%, at least 80%, at least 85%, at least 90% or at least 95% of the CD3+ T cells in the population of lymphocytes are viable as determined with a cell counter, in particular with a NucleoCounter NC-202.

Viability may further be determined by trypan blue cell counting as known in the art. That is, in certain embodiments, at least 70%, at least 75%, at least 80%, at least 85%, at least 90% or at least 95% of the CD3+ T cells in the population of lymphocytes are viable as determined by trypan blue cell counting.

It is to be understood that viability will differ depending on the method with which it is determined, in particular due to variations in the expression of cell markers such as AnnV. It is thus sufficient if viability of at least 70%, at least 75%, at least 80%, at least 85%, at least 90% or at least 95% of the CD3+ T cells in the population of lymphocytes can be achieved with at least one suitable method known in the art, preferably one of the methods disclosed herein.

Further, it is preferred that at least 50% of the CD3+ T cells in the population of lymphocytes are CD27 and/or CD28 positive cells. In certain embodiments, at least 55%, at least 60%, at least 65%, at least 70%, at least 75%, at least 80%, at least 85%, at least 90% or at least 95% of the T cell portion is CD27 and/or CD28 positive. It is preferred that at least 75% of the T cell portion is CD27 and/or CD28 positive.

Alternatively, at least 20% of the CD3+ T cells in the population of lymphocytes are CD27 and/or CD28 positive cells. In certain embodiments, at least 25%, at least 30%, at least 35%, at least 40%, at least 45% or at least 50% of the T cell portion is CD27 and/or CD28 positive.

CD27 is a member of the tumor necrosis factor receptor superfamily. This receptor is required for generation and long-term maintenance of T cell immunity. It binds to ligand CD70 and plays a key role in regulating B cell activation and immunoglobulin synthesis. CD27 is predominantly expressed in naïve, central memory (CM) and effector memory (EM) T cells but not in terminal effector (TE) T cells.

CD28 is one of the proteins expressed on T cells that provide co-stimulatory signals required for T cell activation and survival. T cell stimulation through CD28 in addition to the T cell receptor (TCR) can provide a potent signal for the production of various interleukins. Similarly to CD27, CD28 is predominantly expressed in naïve, central memory (CM) and effector memory (EM) T cells but not in terminal effector (TE) T cells.

As mentioned above, the T cells in the population of lymphocytes preferably comprise a low number of terminal effector T cells. Accordingly, in certain embodiments, at least 20%, at least 30%, at least 40%, at least 50%, at least 60%, at least 70%, at least 80% or at least 90% of the CD3+ T cells in the population of lymphocytes express the cell surface marker CD27. In other embodiments, at least 20%, at least 30%, at least 40%, at least 50%, at least 60%, at least 70%, at least 80% or at least 90% of the CD3+ T cells in the population of lymphocytes express the cell surface marker CD28. In other embodiments, at least 20%, at least 30%, at least 40%, at least 50%, at least 60%, at least 70%, at least 80% or at least 90% of the CD3+ T cells in the population of lymphocytes express the cell surface markers CD27 and CD28.

The skilled person is aware of methods to determine the percentage of CD27 and/or CD28 positive cells in a population of cells. For example, the percentage of CD27 and/or CD28 positive cells in a population of cells may be determined by flow cytometry. Antibodies directed against CD27 and CD28 are known in the art and are commercially available.

In a particular embodiment, the invention relates to the method according to the invention, wherein less than 10% of said T cell portion are positive for at least one of the markers from the group consisting of: CD45RA, CD57 and KLRG1.

That is, the CD3+ T cells in the population of lymphocytes may further by characterized by the absence of one or more senescence markers.

In certain embodiments, it is preferred that less than 10%, less than 9%, less than 8%, less than 7%, less than 6% or less than 5% of the CD3+ T cells in the population of lymphocytes are positive for the cell surface marker CD45RA.

The term “CD45RA”, as used herein, refers to isoform RA of the cluster of differentiation 45, or protein tyrosine phosphatase, receptor type, C (PTPRC). CD45RA, preferably in combination with CD57 and KLRG1, is widely accepted as a marker for terminal differentiation of CD8+ memory T cells. The percentage of CD45RA positive cells in a population of lymphocytes is preferably determined by flow cytometry using antibodies directed against CD45RA.

In certain embodiments, it is preferred that less than 10%, less than 9%, less than 8%, less than 7%, less than 6% or less than 5% of the CD3+ T cells in the population of lymphocytes are positive for the cell surface marker CD57.

The CD57 antigen (alternatively HNK-1, LEU-7, or L2) is routinely used to identify terminally differentiated ‘senescent’ cells with reduced proliferative capacity and altered functional properties. The percentage of CD57 positive cells in a population of lymphocytes is preferably determined by flow cytometry using antibodies directed against CD57.

In certain embodiments, it is preferred that less than 10%, less than 9%, less than 8%, less than 7%, less than 6% or less than 5% of the CD3+ T cells in the population of lymphocytes are positive for the cell surface marker KLRG1.

Killer cell lectin-like receptor subfamily G member 1 (KLRG1) is a protein that in humans is encoded by the KLRG1 gene. KLRG1 is expressed on NK cells and antigen-experienced T cells and has been postulated to be a marker of senescence. The percentage of KLRG1 positive cells in a population of lymphocytes is preferably determined by flow cytometry using antibodies directed against KLRG1.

In certain embodiments, less than 10%, less than 9%, less than 8%, less than 7%, less than 6% or less than 5% of the CD3+ T cells in the population of lymphocytes are positive for at least one of the cell surface markers CD45RA, CD57 and/or KLRG1. In certain embodiments, less than 10%, less than 9%, less than 8%, less than 7%, less than 6% or less than 5% of the CD3+ T cells in the population of lymphocytes are positive for two of the cell surface markers CD45RA, CD57 and/or KLRG1. In certain embodiments, less than 10%, less than 9%, less than 8%, less than 7%, less than 6% or less than 5% of the CD3+ T cells in the population of lymphocytes are positive for all three of the cell surface markers CD45RA, CD57 and/or KLRG1.

In certain embodiments, less than 10%, less than 9%, less than 8%, less than 7%, less than 6% or less than 5% of the CD3+ T cells in the population of lymphocytes are positive for at least one of the cell surface markers CD45RA and/or CD57. In certain embodiments, less than 10%, less than 9%, less than 8%, less than 7%, less than 6% or less than 5% of the CD3+ T cells in the population of lymphocytes are double positive for CD45RA and CD57.

In certain embodiments, less than 10%, less than 9%, less than 8%, less than 7%, less than 6% or less than 5% of the CD3+ T cells in the population of lymphocytes are positive for at least one of the cell surface markers KLRG1 and/or CD57. In certain embodiments, less than 10%, less than 9%, less than 8%, less than 7%, less than 6% or less than 5% of the CD3+ T cells in the population of lymphocytes are double positive for KLRG1 and CD57.

In certain embodiments, more than 50%, more than 60%, more than 70%, more than 80%, more than 85%, more than 90% or more than 95% of the CD3+ T cells in the population of lymphocytes are negative for at least one of the cell surface markers CD45RA, CD57 and/or KLRG1. In certain embodiments, more than 50%, more than 60%, more than 70%, more than 80%, more than 85%, more than 90% or more than 95% of the CD3+ T cells in the population of lymphocytes are negative for two of the cell surface markers CD45RA, CD57 and/or KLRG1. In certain embodiments, more than 80%, more than 85%, more than 90% or more than 95% of the CD3+ T cells in the population of lymphocytes are double negative for CD57 and KLRG1. In certain embodiments, more than 80%, more than 85%, more than 90% or more than 95% of the CD3+ T cells in the population of lymphocytes are triple negative for CD45RA, CD57 and KLRG1.

In a particular embodiment, the invention relates to a population of lymphocytes comprising at least 90% CD3+ T cells and less than 5% B cells, wherein at least 70% of said T cell portion are viable, at least 50% are CD27/CD28 double positive and at least 80% are double negative for CD57 and KLRG1.

In a particular embodiment, the invention relates to the population of lymphocytes according to the invention, wherein said T cell portion has an average telomere length of at least 5 kb, at least 6 kb, at least 7 kb, at least 8 kb, at least 9 kb. The skilled person is aware that the average telomere length depends on the starting material. For example, the average telomere length depends on the age of the patient from which the starting material has been obtained.

Alternatively, or in addition to the senescence markers CD45RA, CD57 and KLRG1, the population of lymphocytes according to the invention may be characterized based on the average telomere length of the CD3+ T cells comprised in the population of lymphocytes. It is known in the art that the onset of replicative senescence is regulated by the length of telomeres, which are specialized structures at chromosome ends that progressively become shorter with each DNA replication cycle. Shortening of telomeres beyond a critical length induces p53-mediated growth arrest and senescence.

Methods for determining the average telomere length in the cells of a cell population are known in the art and have been described for example by Huang et al. (Scientific Reports volume 7, Article number: 6785 (2017)).

In certain embodiments, the CD3+ T cells in the population of lymphocytes may be characterized by an average telomere length of at least 5 kb, at least 6 kb, at least 7 kb, at least 8 kb, at least 9 kb or at least 10 kb.

Instead of characterizing the lymphocytes based on the average length of all telomeres of the CD3+ T cells comprised in the population of lymphocytes, the CD3+ T cells may also be characterized based on the average length of the shortest 20% of telomeres. That is, in certain embodiments, the invention relates to the population of lymphocytes according to the invention, wherein the shortest 20% of telomeres in said T cell portion has an average telomere length of at least 1 kb, at least 1.5 kb, at least 2 kb, at least 2.5 kb, at least 3 kb.

In a particular embodiment, the invention relates to the population of lymphocytes according to the invention, wherein less than 10% of said T cell portion secrete at least one protein from the group consisting of: TNF-α, IL-4, IL-5, Granzyme B and Perforin.

In certain embodiments, the invention relates to the population of lymphocytes according to the invention, wherein less than 15% of said T cell portion secrete at least one protein from the group consisting of: TNF-α, IL-4 and IL-5.

In certain embodiments, the invention relates to the population of lymphocytes according to the invention, wherein less than 10% of said T cell portion secrete at least one protein from the group consisting of: TNF-α, IL-4 and IL-5.

In certain embodiments, the invention relates to the population of lymphocytes according to the invention, wherein less than 15% of said T cell portion secrete TNF-α and wherein less than 10% of said T cell portion secrete at least one protein from the group consisting of: IL-4 and IL-5.

Alternatively, or in addition to the senescence markers CD45RA, CD57 and KLRG1, the population of lymphocytes according to the invention may be characterized based on the secretion profile of the CD3+ T cells comprised in the population of lymphocytes. It is known in the art that terminal effector T cells secrete different proteins than less differentiated T cells. Accordingly, senescence of the cells in a population of cells may be determined based on the proteins that are secreted by the cells in the population.

In certain embodiments, the CD3+ T cells in the population of lymphocytes may be characterized in that less than 15% of these CD3+ T cells secrete TNF-α. In certain embodiments, the CD3+ T cells in the population of lymphocytes may be characterized in that less than 1% of these CD3+ T cells secrete TNF-α. Tumor necrosis factor (TNF, cachexin, or cachectin; often called tumor necrosis factor alpha or TNF-α) is a cytokine—a small protein used by the immune system for cell signaling. TNF-α is predominantly secreted by terminal effector T cells, but not by naïve T cells and central memory T cells.

In certain embodiments, less than 15%, less than 10%, less than 9%, less than 8%, less than 7%, less than 6% or less than 5% of the CD3+ T cells in the population of lymphocytes secrete detectable mounts of TNF-α.

In certain embodiments, the CD3+ T cells in the population of lymphocytes may be characterized in that less than 15% of these CD3+ T cells secrete IL-4. In certain embodiments, the CD3+ T cells in the population of lymphocytes may be characterized in that less than 10% of these CD3+ T cells secrete IL-4. Interleukin 4 (IL-4) has many biological roles, including the stimulation of activated B cell and T cell proliferation, and the differentiation of B cells into plasma cells. IL-4 is predominantly secreted by terminal effector T cells, but not by naïve T cells and central memory T cells.

In certain embodiments, less than 15%, less than 10%, less than 9%, less than 8%, less than 7%, less than 6% or less than 5% of the CD3+ T cells in the population of lymphocytes secrete detectable mounts of IL-4.

In certain embodiments, the CD3+ T cells in the population of lymphocytes may be characterized in that less than 15% of these CD3+ T cells secrete IL-5. In certain embodiments, the CD3+ T cells in the population of lymphocytes may be characterized in that less than 10% of these CD3+ T cells secrete IL-5. Through binding to the interleukin-5 receptor, interleukin 5 stimulates B cell growth and increases immunoglobulin secretion—primarily IgA. It is also a key mediator in eosinophil activation. IL-5 is predominantly secreted by terminal effector T cells, but not by naïve T cells and central memory T cells.

In certain embodiments, less than 15%, less than 10%, less than 9%, less than 8%, less than 7%, less than 6% or less than 5% of the CD3+ T cells in the population of lymphocytes secrete detectable mounts of IL-5.

In certain embodiments, the CD3+ T cells in the population of lymphocytes may be characterized in that less than 10% of these CD3+ T cells secrete Granzyme B. Granzyme B (GrB) is a serine protease most commonly found in the granules of natural killer cells (NK cells) and cytotoxic T cells. It is secreted by these cells along with the pore forming protein perforin to mediate apoptosis in target cells. Granzyme B is predominantly secreted by terminal effector T cells and effector memory T cells, but not by naïve T cells and central memory T cells.

In certain embodiments, less than 10%, less than 9%, less than 8%, less than 7%, less than 6% or less than 5% of the CD3+ T cells in the population of lymphocytes secrete detectable mounts of Granzyme B.

In certain embodiments, the CD3+ T cells in the population of lymphocytes may be characterized in that less than 10% of these CD3+ T cells secrete Perforin. Perforin is a pore forming cytolytic protein found in the granules of cytotoxic T lymphocytes (CTLs) and natural killer cells (NK cells). Upon degranulation, perforin binds to the target cell's plasma membrane, and oligomerizes in a Ca2+ dependent manner to form pores on the target cell. The pore formed allows for the passive diffusion of a family of pro-apoptotic proteases, known as the granzymes, into the target cell. Perforin is predominantly secreted by terminal effector T cells, but not by naïve T cells and central memory T cells.

In certain embodiments, less than 10%, less than 9%, less than 8%, less than 7%, less than 6% or less than 5% of the CD3+ T cells in the population of lymphocytes secrete detectable mounts of Perforin.

In certain embodiments, larger fractions of the cell population may express Granzyme B and/or Perforin. It is thus preferred herein, that less than 15% or, more preferably, less than 10% of the T cell portion comprised in the population of cells secrete at least one protein, at least two proteins or all proteins from the group consisting of: TNF-α, IL-4 and IL-5.

In certain embodiments, the invention relates to the population of lymphocytes according to the invention, wherein less than 10% of said T cell portion secrete at least one protein from the group consisting of: TNF-α, IL-4, IL-5, Granzyme B and Perforin. In certain embodiments, the invention relates to the population of lymphocytes according to the invention, wherein less than 10% of said T cell portion secrete two of the proteins from the group consisting of: TNF-α, IL-4, IL-5, Granzyme B and Perforin. In certain embodiments, the invention relates to the population of lymphocytes according to the invention, wherein less than 10% of said T cell portion secrete three of the proteins from the group consisting of: TNF-α, IL-4, IL-5, Granzyme B and Perforin. In certain embodiments, the invention relates to the population of lymphocytes according to the invention, wherein less than 10% of said T cell portion secrete four of the proteins from the group consisting of: TNF-α, IL-4, IL-5, Granzyme B and Perforin. In certain embodiments, the invention relates to the population of lymphocytes according to the invention, wherein less than 10% of said T cell portion secrete all of the proteins from the group consisting of: TNF-α, IL-4, IL-5, Granzyme B and Perforin.

In certain embodiments, the invention relates to the population of lymphocytes according to the invention, wherein less than 10% of said T cell portion secrete at least one of the proteins from the group consisting of: TNF-α, IL-4 and IL-5. In certain embodiments, the invention relates to the population of lymphocytes according to the invention, wherein less than 10% of said T cell portion secrete at least two of the proteins from the group consisting of: TNF-α, IL-4 and IL-5. In certain embodiments, the invention relates to the population of lymphocytes according to the invention, wherein less than 10% of said T cell portion secrete all of the proteins from the group consisting of: TNF-α, IL-4 and IL-5.

Within the present invention, a cell is determined to secrete a specific protein, if detectable amounts of said protein can be identified in an ELISpot assay. The enzyme-linked immunospot (ELISpot) assay is a highly sensitive immunoassay that measures the frequency of cytokine-secreting cells at the single-cell level. In this assay, cells are cultured on a surface coated with a specific capture antibody in the presence or absence of stimuli. Proteins, such as cytokines, that are secreted by the cells will be captured by the specific antibodies on the surface. After an appropriate incubation time, cells are removed and the secreted molecule is detected using a detection antibody in a similar procedure to that employed by the ELISA. The detection antibody is either biotinylated and followed by a streptavidin-enzyme conjugate or the antibody is directly conjugated to an enzyme. By using a substrate with a precipitating rather than a soluble product, the end result is visible spots on the surface. Each spot corresponds to an individual cytokine-secreting cell. The ELISpot assay captures the presence of cytokines immediately after secretion, in contrast to measurements that are skewed by receptor binding or protease degradation. The assay is considered as one of the most sensitive cellular assays available. The limit of detection typically achieved can be 1 in 100,000 cells. The high sensitivity of the assay makes it particularly useful for studies of the small population of cells found in specific immune responses. ELISpot assays for determining the percentage of cells that secrete IFN-γ, TNF-α, IL-4, IL-5, Granzyme B and Perforin are known in the art.

Alternatively or in addition, the secretion of these proteins can be approximated by flow cytometry. For this, T cells have to be fixated and permeabilized such that antibodies can be used for quantifying the intracellular pools of the respective proteins. Methods for quantifying the intracellular pools of TNF-α, IL-4, IL-5, Granzyme B and/or Perforin are known in the art.

In a particular embodiment, the invention relates to the population of lymphocytes according to the invention, wherein at least 50%, at least 60%, at least 70%, at least 80% or at least 90% of the T cells in said T cell portion are CD8+ T cells.

That is, it is preferred that the majority of T cells in the population of lymphocytes are CD8+ T cells. As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions. As used herein, the term “tumor-infiltrating CD8+ T cell” refers to the pool of CD8+ T cells of the patient that have left the blood stream and have migrated into a tumor.

Preferably, the second largest portion of T cells in the population of lymphocytes are CD4+ T cells. As used herein, the term “CD4+ T cell” refers to a T cell that presents the co-receptor CD4 on its surface. CD4 is a transmembrane glycoprotein that serves as a co-receptor for T cell receptor (TCR), which can recognize a specific antigen. In certain embodiments, CD4+ T cells are T helper cells. T helper cells (TH cells) assist other white blood cells in immunologic processes, including maturation of B cells into plasma cells and memory B cells, and activation of cytotoxic T cells and macrophages. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including THI, TH2, TH3, TH17, TH9, or TFH, which secrete different cytokines to facilitate different types of immune responses. Signaling from the APC directs T cells into particular subtypes. In certain embodiments, CD4+ T cells are regulatory T cells. Regulatory T cells play an essential role in the dampening of immune responses, in the prevention of autoimmune diseases and in oral tolerance.

In certain embodiments, the invention relates to the population of lymphocytes according to the invention, wherein up to 50%, up to 40%, up to 30%, up to 20% or up to 10% of the T cells in said T cell portion are CD4+ T cells.

In certain embodiments, the invention relates to the population of lymphocytes according to the invention, wherein the ratio between CD8+ T cells and CD4+ T cells in said T cell portion is between 1:1 and 10:1. In certain embodiments, the invention relates to the population of lymphocytes according to the invention, wherein the ratio between CD8+ T cells and CD4+ T cells in said T cell portion is 1:1, 2:1, 3:1, 4:1, 5:1, 6:1, 7:1, 8:1, 9:1 or 10:1 or more than 10:1.

The skilled person is aware of methods to determine the percentage of CD4+ and/or CD8+ T cells in a population of lymphocytes. For example, the percentage of CD4+ and/or CD8+ T cells in a population of lymphocytes may be determined by flow cytometry using antibodies directed against CD4 and/or CD8, respectively.

In a particular embodiment, the invention relates to the population of lymphocytes according to the invention, wherein at least two T cells in said T cell portion are directed against different antigens.

That is, the T cells comprised in the population of lymphocytes preferably recognize more than one antigen. Obtaining the population of lymphocytes according to the invention comprises a step of contacting these lymphocytes with a pool of different antigenic peptides. Thus, it is envisioned that primarily the T cells that recognize an antigen from the pool of antigens are expanded. The pool of antigenic peptides may comprise more than 50, more than 100, more than 200, more than 300, more than 400, more than 500 or more than 1000 different antigenic peptides. Accordingly, in certain embodiments, the T cell portion comprised in the population of lymphocytes may comprise at least 2, at least 5, at least 10, at least 25, at least 50, at least 75, at least 100, at least 150, at least 200 or at least 300 T cells, wherein each T cell is directed against a different antigen. Non-limiting examples of antigens that may be recognized by the T cells comprised in the population of lymphocytes are provided herein.

It is preferred that that the population of lymphocytes comprises a number of cells that is suitable for use in adoptive cell transfer (ACT) therapy in humans. That is, the population of lymphocytes according to the invention comprises at least 10⁶, 107, 10⁸, 109 of 10¹⁰ CD3+ T cells. Preferably, the population of lymphocytes according to the invention comprises between 10⁶ and 10¹⁰ CD3+ T cells, preferably between 107 and 109 T cells. In certain embodiments, the population of lymphocytes according to the invention comprises at least 10×10⁸ T cells.

In a particular embodiment, the invention relates to a population of lymphocytes for allogenic cell transfer in humans, the population of lymphocytes comprising at least 90% CD3+ T cells and less than 5% B cells, wherein at least 70% of said T cell portion are viable, at least 50% are CD27/CD28 double positive and less than 10% are triple positive for CD45RA, CD57 and KLRG1.

In a particular embodiment, the invention relates to a population of lymphocytes for allogenic cell transfer in humans, the population of lymphocytes comprising at least 90% CD3+ T cells and less than 5% B cells, wherein at least 70% of said T cell portion are viable, at least 20% are CD27/CD28 double positive and less than 10% are triple positive for CD45RA, CD57 and KLRG1.

In a particular embodiment, the invention relates to a population of lymphocytes for adoptive cell transfer therapy in humans, the population of lymphocytes comprising at least 90% CD3+ T cells and less than 5% B cells, wherein at least 70% of said T cell portion are viable, at least 50% are CD27/CD28 double positive and less than 10% are positive for CD45RA and CD57.

In a particular embodiment, the invention relates to a population of lymphocytes for adoptive cell transfer therapy in humans, the population of lymphocytes comprising at least 90% CD3+ T cells and less than 5% B cells, wherein at least 70% of said T cell portion are viable, at least 20% are CD27/CD28 double positive and less than 10% are positive for CD45RA and CD57.

In a particular embodiment, the invention relates to a population of lymphocytes for adoptive cell transfer therapy in humans, the population of lymphocytes comprising at least 90% CD3+ T cells and less than 5% B cells, wherein at least 70% of said T cell portion are viable, at least 50% are CD27/CD28 double positive and less than 10% are positive for CD57 and KLRG1.

In a particular embodiment, the invention relates to a population of lymphocytes for adoptive cell transfer therapy in humans, the population of lymphocytes comprising at least 90% CD3+ T cells and less than 5% B cells, wherein at least 70% of said T cell portion are viable, at least 20% are CD27/CD28 double positive and less than 10% are positive for CD57 and KLRG1.

In a particular embodiment, the invention relates to a population of lymphocytes for adoptive cell transfer therapy in humans, the population of lymphocytes comprising at least 90% CD3+ T cells and less than 5% B cells, wherein at least 70% of said T cell portion are viable, at least 50% are CD27/CD28 double positive and more than 80% are negative for CD57 and KLRG1.

In a particular embodiment, the invention relates to a population of lymphocytes for adoptive cell transfer therapy in humans, the population of lymphocytes comprising at least 90% CD3+ T cells and less than 5% B cells, wherein at least 70% of said T cell portion are viable, at least 20% are CD27/CD28 double positive and more than 80% are negative for CD57 and KLRG1.

Preferably, the population of lymphocytes according to the invention is suitable for use in autologous cell therapy. Autologous cell therapy is a therapeutic intervention that uses an individual's cells, which are cultured and expanded outside the body, and reintroduced into the donor. Advantages of such an approach include the minimization of risks from systemic immunological reactions, bio-incompatibility, and disease transmission associated with non-autologous grafts or cells from the individual. It is preferred that the cells comprised in the population of lymphocytes according to the invention have been obtained by expanding T cells of an individual ex vivo and are subsequently infused back into the same individual.

Thus, in a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 50% are CD27/CD28 double positive and less than 10% are triple positive for CD45RA, CD57 and KLRG1.

Thus, in a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 20% are CD27/CD28 double positive and less than 10% are triple positive for CD45RA, CD57 and KLRG1.

In a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 50% are CD27/CD28 double positive and less than 10% are double positive for CD45RA and CD57.

In a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 20% are CD27/CD28 double positive and less than 10% are double positive for CD45RA and CD57.

In a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 50% are CD27/CD28 double positive and less than 10% are double positive for CD57 and KLRG1.

In a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 20% are CD27/CD28 double positive and less than 10% are double positive for CD57 and KLRG1.

In a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 50% are CD27/CD28 double positive and more than 80% are double negative for KLRG1 and CD57.

In a particular embodiment, the invention relates to a population of lymphocytes for autologous cell therapy in humans, the population of lymphocytes comprising at least 90% CD3+ T cells and less than 5% B cells, wherein at least 70% of said T cell portion are viable, at least 20% are CD27/CD28 double positive and more than 80% are double negative for KLRG1 and CD57.

The invention also provides methods for producing a population of lymphocytes specific for one or more antigens as defined herein, said method comprising a single culture phase, wherein said single phase comprises (a) culturing a tissue or blood sample from a subject in the presence of said one or more antigens, which sample is known or suspected to contain lymphocytes; or (b)culturing the lymphocytes in the presence of said one or more antigens, which lymphocytes are isolated from a tissue or blood sample from a subject.

In certain embodiments, said culturing is continued until said T cell population of at least 10×10⁸ cells is achieved. At all times the lymphocytes and/or population of T cells are maintained at temperatures greater than 0° C. during said single culture phase.

In certain embodiments, said culturing is continued until said T cell population of at least 1×10⁷ cells is achieved. At all times the lymphocytes and/or population of T cells are maintained at temperatures greater than 0° C. during said single culture phase.

In certain embodiments, the sample comprising the lymphocytes and/or the T cells population are maintained at temperatures greater than 0° C. subsequent to isolation from the subject and prior to culture. However, it is to be understood that frozen samples may also be used in the method of the present invention.

Previous expansion protocols for autologous tumor-infiltrating lymphocytes (TILs) consist of two phases. In an initial pre-REP phase, TILs are expanded for 3-5 weeks. In a subsequent REP phase, the TILs obtained in the pre-REP phase are rapidly expanded for an additional two weeks. Between the pre-REP and the REP phase, the TILs are typically cryopreserved. The disadvantage of this long cultivation period, including the optional cryopreservation step, is that a large fraction of the lymphocytes in the final product are terminal effector cells which rapidly die after infusion into the patient.

It is thus an aim of the present invention to establish an expansion protocol for lymphocytes with which high cell numbers (10⁷ cells and more) can be reached in two to eight weeks, preferably in two to six weeks, more preferably in two to four weeks, and without the need of a cryopreservation step. Thus, in a preferred embodiment, the lymphocytes are kept at temperatures greater than 0° C. throughout the entire culturing process.

The method of the present invention is characterized in that the cells are cultured in a “conditioned culture medium”. That is, certain parameters of the culture medium are monitored throughout the entire process and are adjusted to predefined values if necessary. Suitable parameters of the culture medium that are monitored and/or adjusted throughout the method of the invention are disclosed elsewhere herein. With that, optimal growth conditions can be maintained throughout the entire process.

The method of the invention is further characterized in that it comprises a step of “dynamic culturing”. Dynamic culturing requires that the cells are cultured in a continuous flow of culture medium. Dynamic culturing comprises both circulation, where conditioned culture medium is circulated withing the growth chamber, and perfusion, where culture medium from the growth chamber is replaced with fresh culture medium.

The methods of expansion of the desired T cell populations from the sample, e.g. comprising lymphocytes and/or T cells, comprises the presentation of one or more antigens to the T cells within the sample to be cultured. The antigens may be presented by any means known in the art and/or described herein suitable to induce expansion of the T cells specifically recognizing the one or more antigens. As an exemplary non-limiting example, the one or more soluble antigens can be continuously provided in the culture medium (e.g. to maintain a steady state concentration or a desired range of concentration(s)) or may be included for one or more specific periods less than the entire culture phase. The soluble antigens can also be introduced at one or more discreet time points of the culture phase. Additionally or alternatively, the soluble antigens can be presented to the lymphocyte samples and/or T cells during the culture by antigen-presenting cells (APCs) as is disclosed herein. It is preferred that the APCs are B cells. The APCs can be engineered to present the one or more desired antigens by any means known in the art or described herein. Alternatively or in addition, The APCs can be contacted with antigenic peptides by any means known in the art or described herein.

In certain embodiments, the one or more antigens that are added in the culturing step are comprised in a tumor sample. That is, the tumor sample itself may simultaneously serve as a source of lymphocytes and as a source of antigens. In such embodiments, the tumor samples may be co-cultured with an APC in the absence of antigenic peptides.

The APCs may be recombinantly engineered to express the one or more antigens of interest either transiently or constitutively. Recombinant engineering can be achieved by any means known in the art or described herein and, preferably, is achieved by transduction using a viral vector or transfection using plasmids or mRNAs.

However, it is preferred herein that the APCs, in particular the B cells, are contacted with antigenic peptides that have been chemically synthesized, as described in more detail below.

The antigens may be one or more known antigen characterizing a disease or cancer, or may be determined by assessing a patient sample to determine one or more neoantigens. For that, patient cells may be collected by biopsy and analyzed by mass spectrometry or scRNAseq to identify the neoantigens. The sequences available from these methods may then be analyzed using a proprietary algorithm to identify and select the relevant neoantigens.

The population of lymphocytes, isolated lymphocytes and/or the methods of their production and use are provided not only as tools for the treatment of disease (e.g. for use as a medicament or in the development and manufacture of a medicament) but will be also be understood to have applicability as model systems for investigating disease therapies. Accordingly, while the lymphocytes of the invention as disclosed herein are preferably human lymphocytes, more preferably primary human lymphocytes (e.g. including NK cells and T cells), and most preferably primary human T cells (e.g. including CD3+ T cells, CD4+ T cells, CD8+ T cells, γδ T cells), also provided are lymphocytes derived from lymphocyte cell lines (whether of human or non-human origin) as well as lymphocytes that are primary cells of non-human origin, for example and not being limited to, primary lymphocytes and lymphocytes derived from mice, rats, monkeys, apes, cats and dogs.

From the more preferred primary human lymphocytes, the most preferred is a primary human T cell. Therefore, the invention also provides populations of primary human T cells characterized by at least 90% CD3+ T cells and less than 5% B cells, wherein at least 70% of said T cell portion are viable, at least 50% are CD27/CD28 double positive and less than 10% are triple positive for CD45RA, CD57 and KLRG1.

Alternatively, the invention also provides populations of primary human T cells characterized by at least 90% CD3+ T cells and less than 5% B cells, wherein at least 70% of said T cell portion are viable, at least 20% are CD27/CD28 double positive and less than 10% are triple positive for CD45RA, CD57 and KLRG1.

Further, the invention also provides populations of primary human T cells characterized by at least 90% CD3+ T cells and less than 5% B cells, wherein at least 70% of said T cell portion are viable, at least 50% are CD27/CD28 double positive and more than 80% are double positive for CD57 and KLRG1.

Further, the invention also provides populations of primary human T cells characterized by at least 90% CD3+ T cells and less than 5% B cells, wherein at least 70% of said T cell portion are viable, at least 20% are CD27/CD28 double positive and more than 80% are double positive for CD57 and KLRG1.

The lymphocyte population provided herein or produced according to the methods provided herein, whether human or not and whether primary or not, can be comprised of any lymphocyte class or subclass known in the art or described herein known or believed useful for adoptive cell therapies and/or known or believed to be of use in an in vitro or in vivo model systems. Non-limiting examples of lymphocyte classes encompassed by the invention include populations of lymphocytes comprising T cells (include CD3+ T cells, CD4+ T cells, CD8+ T cells, γδ T cells, invariant T cells), as well as B cells, Macrophages, and NK cells, as well as combinations thereof.

The populations of cells provided herein and/or for use in the methods of their production (e.g. APCs, preferably B cells) includes genetically engineered cells that may either be a directly genetically engineered cell, i.e. a cell that has been directly subject to genetic engineering methods, or may be a cell derived from such an engineered cell, e.g. a daughter cell or progeny of a cell that was directly genetically engineered. Any suitable genetic engineering method can be used, including but not limited to lipofection, CRISPR/CAS, calcium phosphate transfection, sleeping beauty transposons, PEG mediated transfection, and transduction with viral vectors (e.g. lentiviral vectors). Exogenous nucleic acid molecules may be introduced into cells as linear molecules and/or as circular molecules (e.g. plasmids, miniplasmids or mRNAs). In non-limiting embodiments, one or more of the lymphocytes within the lymphocyte population of the invention can be engineered to express one or more immunomodulators such as OX40L, 4-1BBL, CD80, CD86, CD83, CD70, CD40L, GITR-L, CD127L, CD30L (CD153), LIGHT, BTLA, ICOS-L (CD275), SLAM (CD150), CD662L, interleukin-12, interleukin-7, interleukin-15, interleukin-17, interleukin-21, interleukin-4, Bc16, Bcl-XL, BCL-2, MCL1, STAT-5, and/or activators of one or more signaling pathways (e.g. the JAK/STAT pathway, the Akt/PBK signaling pathway, the BCR signaling pathway, and/or the BAFF/BAFFR signaling pathway). Similarly, one or more APC of use in the methods disclosed herein may be engineered to express one or more known antigens or one or more neoantigens determined from a patient sample.

It is preferred that the APCs, in particular the B cells, are engineered to express one or more of the immunomodulators OXO40L, 4-1BB and/or interleukin-12.

In certain embodiments, the APCs, in particular the B cells, are engineered to express OXO40L and 4-1BB.

In certain embodiments, the APCs, in particular the B cells, are engineered to express OXO40L and interleukin-12.

In certain embodiments, the APCs, in particular the B cells, are engineered to express 4-1BB and interleukin-12.

In certain embodiments, the APCs, in particular the B cells, are engineered to express OXO40L, 4-1BB and interleukin-12.

Nucleic acids encoding the above-mentioned immunomodulators may be introduced into the APC's, in particular the B cells, by any method known in the art and/or disclosed herein. Preferably, mRNAs encoding the above-mentioned immunomodulators are introduced into the APC's, in particular the B cells, by means of transfection to transiently express the encoded proteins.

The lymphocytes and populations of lymphocytes of the invention, preferably human lymphocytes, more preferably primary human lymphocytes, and most preferably primary human T cells, are envisioned for use in therapy and may be autologous (i.e., the donor from which the cells were derived and recipient are the same subject) or may be allogenic (i.e., the donor from which the cells were derived is different from the recipient). Where autologous, any appropriate source can be used as known in the art or described herein, including but not limited to a tumor environment whether solid (such as for tumor-infiltrating lymphocytes (TILs)) or circulating tumor cells; and peripheral blood (such as for PBMCs). Preferably, the lymphocytes in the population of lymphocytes have been obtained by expanding TILs ex vivo.

Where the cells are allogenic, they may be further genetically engineered or prepared such that they are not alloreactive. As understood in the art, and as used herein, non-alloreactive indicates that the cells have been engineered (e.g., genetically engineered) such that they are rendered incapable of being recognized as or recognizing allogenic cells (cells of foreign origin). Similarly, the genetically engineered lymphocytes of the invention can be additionally or alternatively engineered so as to prevent their own recognition by the recipient's immune system. As a non-limiting example in this respect, the lymphocytes of the invention may have disruption or deletion of the endogenous major histocompatibility complex (MHC). Such cells may have diminished or eliminated expression of the endogenous MHC, preventing or diminishing activation of the recipient's immune system against the autologous cells.

As understood in the art, such non-alloreactive cells are incapable of reacting to cells of a foreign host. Therefore, non-alloreactive cells derived from third-party donors may become universal, i.e. recipient independent. As explained above, the non-alloreactive cells may also comprise additional engineering rendering them incapable of eliciting an immune response and/or of being recognized by the recipient's immune system, preventing them from being rejected. Such cells that are non-alloreactive and/or that are incapable of eliciting an immune response or being recognized by the recipient's immune system may also be termed “off the shelf” cells as is known in the art. Lymphocytes can be rendered non-alloreactive and/or incapable of eliciting or being recognized by an immune system by any means known in the art or described herein. In a non-limiting example, in the context of T cells non-alloreactive cells can have reduced or eliminated expression of the endogenous T cell receptor (TCR) when compared to an unmodified control cell. Such non-alloreactive T cells may comprise modified or deleted genes involved in self-recognition, such as but not limited to, those encoding components of the TCR including, for example, the alpha and/or beta chain. Similarly, the genetically engineered lymphocytes disclosed herein can additionally or alternatively have reduced or eliminated expression of the endogenous MHC when compared to an unmodified control cell. Such lymphocytes may comprise any modifications or gene deletions known in the art or described herein to minimize or eliminate antigen presentation, in particular, so as to avoid immunogenic surveillance and elimination in the recipient. As noted, non-alloreactive cells which optionally avoid immune surveillance are widely referenced in the art as “off the shelf” cells and the terms are used interchangeably herein. Such non-alloreactive/off the shelf leucocytes may be obtained from repositories. The genetic modifications to reduce or eliminate alloreactivity (i.e. to render the cell non-alloreactive) and/or to reduce or eliminate self-antigen presentation (i.e. so as to prevent them from eliciting an immune response or being recognized by the recipient's immune system), as known in the art or described herein can be performed before, concurrently with, or subsequent to any other genetic engineering in the context of the present invention.

The invention also encompasses a population of lymphocytes, preferably human lymphocytes obtainable by any method disclosed herein.

The invention provides a method of immunotherapy for treating a disease comprising the use of the cells or population of cells as disclosed herein. Accordingly, provided is a population of lymphocytes (preferably human leucocytes, more preferentially primary human lymphocytes, and most preferentially primary human T cells) as described herein for use as a medicament. The invention also provides the population of lymphocytes as disclosed herein within a pharmaceutically acceptable carrier in the form of a pharmaceutical composition. The medicament and pharmaceutical compositions as disclosed herein are, in particular, of use in adoptive cell therapies.

The population of lymphocytes, medicaments and/or pharmaceutical compositions of the invention are of use in the treatment of cancers regardless of tumor type, as well as in the treatment of viral diseases, bacterial diseases such as Tuberculosis (including antibiotic-resistant diseases), and parasitic diseases.

The population of lymphocytes, medicaments and/or pharmaceutical compositions of the invention can be used in combination with antineoplastic or immunomodulating agents such as, but not limited to Azacitidine, Capecitabine, Carmofur, Cladribine, Clofarabine, Cytarabine, Decitabine, Floxuridine, Fludarabine, Fluorouracil, Gemcitabine, Mercaptopurine, Nelarabine, Pentostatin, Tegafur, Tioguanine, Methotrexate, Pemetrexed, Raltitrexed, Hydroxycarbamide, Irinotecan, Topotecan, Daunorubicin, Epirubicin, Idarubicin, Mitoxantrone, Valrubicin, Etoposide, Teniposide, Cabazitaxel, Docetaxel, Paclitaxel, Vinblastine, Vincristine, Vindesine, Vinflunine, Vinorelbine, Bendamustine, Busulfan, Carmustine, Chlorambucil, Chlormethine, Cyclophosphamide, Dacababazine, Fotemustine, Ifosfamide, Lomustine, Melphalan, Streptozotocin, Temozolomide, Carboplatin, Cisplatin, Nedaplatin, Oxaliplatin, Altretamine, Bleomycin, Bortezomib, Dactinomycin, Estramustine, Ixabepilone, Mytomycin, Alemtuzumab, Bevacizumab, Cetuximab, Denosumab, Gemtuzumab ozogamicin, Ibritumomab tiuxetan, Ipilimumab, Nivolumab, Ofatumumab, Panitumumab, Pembolizumab, Pertuzumab, Rituximab, Tositumomab, Trastuzumab, Afatinib, Aflibercept, Axitinib, bosutinib, Crizotinib, Dasatinib, Erlotinib, Gefitinib, Imatinib, Lapatinib, Nilotinib, Pazopanib, Ponatinib, Regorafenib, Ruxolitinib, Sorafenib, Sunitinib, Vandetanib, Everolimus, Temsirolimus, Alitretinoin, Bexarotene, Isotretinoin, Tamibarotene, Tretinoin, Lenalidomide, Pomalidomide, Thalidomide, Panobinostat, Romidepsin, Valproate, Vorinostat, Anagrelide, Arsenic trioxide, Aspariganse, BCG Vaccine, Denileukin diftitox, Vemurafenib, goserelin, Toremifene, Fulvestrant, bicalutamide, enzalutamide, apalutamide, darolutamide, anastrozole, letrozole, degarelix, abiraterone, filgrastim, molgramostin, pegfilgrastim, lipecfilgrastim, balugrastim, levacetylmethadol, interferon gamma, interferon alfa-2b, interferon alfa-n1, interferon beta-1a, peginterferon alfa-2b, peginterferonbeta-la, ropeginterferon alfa-2v, tasonermin, histamine dihydrochloride, mifarmurtide, plerixafor, sipuleucel-T, dasiprotimut-T, muronab-CD3, mycophenolic acid, sirolimus, leflunomide, efalizumab, natalizumab, abatacept, exulizumab, ofatumumuab, fingolimd, eltrombopag, tofacitinib, teriflunomide, apremilast, vedolizumab, baricitinib, ozamimod, upacitinib, filgotinib, etanercept, infliximab, adalimumab, certolizumab pegol, golimumab, valdecoxib, anakinra, rilonacept, ustekinumab, tocilizumab, canakinumab, secukinumab, lopinavir, ritonavir, brodalumab, ixekizumab, sarilumab, tacrolimus, voclosporin, thalidomide, methotrexate, lenalidomide, pirfenidone, pomalidomide, dimethyl fumarate, darvadstrocel.

As used herein combination with the population of lymphocytes, medicaments and/or pharmaceutical compositions of the invention does not indicate that the lymphocyte therapy and one or more additional medicaments need be administered together, e.g. in the same infusion. Combination includes concomitant and sequential administration in any order. Combination also includes dosing schemes wherein one or more agent is administered multiple times over the time frame, e.g. of days, weeks, or months, and the other agent or agents is administered only once or in according to a different dosing scheme. Combination includes any scheme wherein the agents are purposefully administered so that the therapeutic effects overlap at least to some extent.

### 3. DETAILED DESCRIPTION

### 3.1 Lymphocytes for Immunotherapy

The invention is in particular directed to a population of lymphocytes (preferably human lymphocytes, more preferably primary human lymphocytes, and most preferably primary human T cells) characterized by at least 90% CD3+ T cells and less than 5% B cells, wherein at least 70% of said T cell portion are viable, at least 50% are CD27/CD28 double positive and less than 10% are triple positive for CD45RA, CD57 and KLRG1. The term “primary” and analogous terms in reference to a cell or cell population as used herein correspond to their commonly understood meaning in the art, i.e. referring to cells that have been obtained directly from living tissue (i.e. a biopsy such as a tumor sample or a blood sample) or from a subject, which cells have not been passaged in culture, or have been passaged and maintained in culture but without immortalization. It is preferred that the primary cells are primary human lymphocytes. Primary cells have undergone very few population doublings, if any.

The population of lymphocytes according to the present invention can comprise any lymphocytes class, subclass, or mixtures thereof as described herein or known in the art to be suitable for use, in particular, in an adoptive cell therapy. However, it is recognized that the methods of the invention may also be applicable for uses outside of therapies, such as in screening methods and/or in model systems, e.g. of use in in vitro assays or in vivo animal models. Non-limiting examples of lymphocytes (which may be primary lymphocytes or derived from cell lines) include NK cells, inflammatory T lymphocytes, cytotoxic T lymphocytes, helper T lymphocytes, CD4+ T lymphocytes, CD8+ T lymphocytes, γδ T lymphocytes, invariant T lymphocytes NK lymphocytes, B lymphocytes and macrophages.

It is preferred herein that at least 50%, at least 60%, at least 70%, at least 80% or at least 90% of the CD3+ T cells comprised in the population of lymphocytes are CD8+ T cells.

### 3.2 Metabolic Characterization

The population of lymphocytes (preferably human lymphocytes, more preferably primary human lymphocytes, and most preferably primary human T cells) may be analyzed for expression of one or more phenotype markers after expansion. In some embodiments, the marker is selected from one or more of TCRab (i.e. TCR.alpha./.beta.), CD57, CD28, CD4, CD27, CD56, CD8a, CD45RA, CD8a, CCR7, CD4, CD3, CD38, CD45RA, and HLA-DR. In some embodiments, expression of one, two, three, four, five, six, seven, eight, nine, ten, eleven, twelve, thirteen, or fourteen markers is examined.

The population of lymphocytes (preferably human lymphocytes, more preferably primary human lymphocytes, and most preferably primary human T cells) may be analyzed for expression of one or more regulatory markers. In some embodiments, the regulatory marker is selected from one or more of CD137, CD8a, Lag3, CD4, CD3, PD-1, TIM-3, CD69, CD8a, TIGIT, CD4, CD3, KLRG1, and CD154.

It is preferred that the population of lymphocytes (preferably human lymphocytes, more preferably primary human lymphocytes, and most preferably primary human T cells) analyzed for expression of both one or more phenotype markers and one or more regulatory markers. Accordingly, the population of lymphocytes (preferably human lymphocytes, more preferably primary human lymphocytes, and most preferably primary human T cells) may be analyzed for expression of one or more of TCRab (i.e. TCR.alpha./.beta.), CD57, CD28, CD4, CD27, CD56, CD8a, CD45RA, CD8a, CCR7, CD4, CD3, CD38, CD45RA, HLA-DR, CD137, CD8a, Lag3, CD4, CD3, PD-1, TIM-3, CD69, CD8a, TIGIT, CD4, CD3, KLRG1, and CD154. It is preferred that at least 50% of the CD3+ T cells comprised in the population of lymphocytes are CD27/CD28 double positive and less than 10% of the CD3+ T cells comprised in the population of lymphocytes are triple positive for CD45RA, CD57 and KLRG1.

Alternatively, it is preferred that at least 50% of the CD3+ T cells comprised in the population of lymphocytes are CD27/CD28 double positive and more than 80% of the CD3+ T cells comprised in the population of lymphocytes are double negative for CD57 and KLRG1.

Preferably, the presence of the above-mentioned markers on the cell surface of the CD3+ T cells comprised in the population of lymphocytes is determined by flow cytometry.

As used herein, the term “flow cytometry” refers to an assay in which the proportion of a material (e.g. lymphocyte comprising a particular maker) in a sample is determined by labeling the material (e.g., by binding a labeled antibody to the material), causing a fluid stream containing the material to pass through a beam of light, separating the light emitted from the sample into constituent wavelengths by a series of filters and mirrors, and detecting the light.

A multitude of flow cytometers are commercially available including for e.g. Becton Dickinson FACScan and FACScaliber (BD Biosciences, Mountain View, CA). Antibodies that may be used for FACS analysis are widely commercially available.

In some embodiments, the viability of the population of lymphocytes (preferably human lymphocytes, more preferably primary human lymphocytes, and most preferably primary human T cells) is at least 70%, at least 75%, at least 80%, at least 85%, at least 90%, at least 95%, or at least 98%. The viability of lymphocytes can be determined by methods known in the art, such as any one of the methods disclosed herein above.

The population of lymphocytes (preferably human lymphocytes, more preferably primary human lymphocytes, and most preferably primary human T cells) can be evaluated for interferon-γ (IFN-γ) secretion in response to stimulation either with an anti-CD3 antibody (such as OKT3) or co-culture with autologous tumor digest or stimulation with antigenic and/or neoantigenic peptides. The skilled person is aware that antigenic and/or neoantigenic peptides have to be presented in an MHC-dependent manner.

In some embodiments, TIL health is measured by IFN-gamma (IFN-γ) secretion. In some embodiments, IFN-γ secretion is indicative of active T cells within the expanded population. In some embodiments, a potency assay for IFN-γ production is employed. IFN-γ production is another measure of cytotoxic potential. IFN-γ production can be measured by determining the levels of the cytokine IFN-γ in the media of the population of lymphocytes (preferably human lymphocytes, more preferably primary human lymphocytes, and most preferably primary human T cells) provided and produced according to the methods herein may be analyzed subsequent to stimulation with antibodies to CD3, CD28, and/or CD137/4-1BB. IFN-γ levels in media from these stimulated population of lymphocytes (preferably human lymphocytes, more preferably primary human lymphocytes, and most preferably primary human T cells) can be determined using by measuring IFN-γ release. In some embodiments, IFN-γ secretion is increased one-fold, two-fold, three-fold, four-fold, or five-fold or more relative to the corresponding cells in the sample prior to expansion.

In some embodiments, telomere length can be used as a measure of cell viability and/or cellular function. In some embodiments, the telomeres are surprisingly the same length in the lymphocyte population produced by the present invention as compared to lymphocyte populations prepared using methods other than those provide herein. Diverse methods have been used to measure the length of telomeres in genomic DNA and cytological preparations. The telomere restriction fragment (TRF) analysis is the gold standard to measure telomere length. However, the major limitation of TRF is the requirement of a large amount of DNA. Two widely used techniques for the measurement of telomere lengths namely, fluorescence in situ hybridization (e.g. FISH; Agilent Technologies, Santa Clara, Calif.) and quantitative PCR can be employed with the present invention. In some embodiments, there is no change in telomere length between the initially harvest lymphocytes of the sample (or any subpopulation thereof, e.g. T cells) and the population of lymphocytes and/or T cells subsequent to expansion.

### 3.3 Lymphocyte Source

The primary lymphocytes described herein can be isolated and/or obtained from a number of tissue sources, including but not limited to, peripheral blood mononuclear cells isolated from a blood sample, bone marrow, lymph node tissue, cord blood, thymus tissue, tissue from a site of infection, ascites, pleural effusion, spleen tissue, and/or tumors by any method known in the art or described herein. It is preferred that the isolated cells and/or samples used in the methods of the present invention, e.g. to generate the populations of lymphocytes (preferably human lymphocytes, more preferably primary human lymphocytes, and most preferably primary human T cells), are obtained from and/or isolated from a population derived from a tumor sample whether solid or circulating (e.g. for the isolation of TILs), or derived from infected tissue (e.g. tissue having a viral, bacterial, or parasitic infection). Methods for isolating/obtaining specific populations of lymphocytes from patients or from donors are well known in the art and include as a first step, for example, isolation/obtaining a donor or patient sample known or expected to contain such cells.

For example, lymphocytes (preferably human lymphocytes, more preferably primary human lymphocytes, and most preferably primary human T cells (including TILs)) may be obtained from a patient tumor sample and then expanded into a larger population. Such expanded cells and/or populations may subsequent to the expansion be optionally cryopreserved for storage and handling prior to administration.

A patient tumor sample may be obtained using methods known in the art, generally via surgical resection, needle biopsy or other means for obtaining a sample that contains a mixture of tumor and lymphocytes. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). It is most preferred that the sample is known to or suspected to contain T cells, in particular TILs. In some embodiments, useful TILs are obtained from malignant melanoma tumors, as these have been reported to have particularly high levels of lymphocytes, in particular, TILs.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the lung, breast, triple negative breast cancer, prostate, colon, rectum, and bladder. In some embodiments, the cancer is selected from cervical cancer, head and neck cancer (including, for example, head and neck squamous cell carcinoma (HNSCC)) glioblastoma, ovarian cancer, sarcoma, pancreatic cancer, bladder cancer, breast cancer, triple negative breast cancer, and non-small cell lung carcinoma. The tissue structure of solid tumors includes interdependent tissue compartments including the parenchyma (cancer cells) and the supporting stromal cells in which the cancer cells are dispersed, which may provide a supporting microenvironment.

After the sample has been isolated/obtained, the desired cells, e.g. human lymphocytes and/or T cells (e.g. TILs), may be cultured under conditions allowing the preferential growth and expansion of desired cell classes, subclasses, or of cells with desired specificities. The methods, in particular, allow the isolation/obtention of populations maintaining stemness and exhibiting low percentages of terminal effector cells, such populations are known in the art to be capable of increased replication and/or high cell killing activity. Such cells are characterized by a high expression of CD27 and CD28, a low expression of CD45RA, CD57 and KLRG1 and a low secretion of TNF-α, IL-4, IL-5, and optionally, Granzyme B and Perforin, as disclosed elsewhere herein.

### 3.4 Antigen Specificity

The present invention provides a method for generating lymphocytes (preferably human lymphocytes, more preferably primary human lymphocytes, and most preferably primary human T cells (such as TILs)) having a defined specificity, i.e. having targeting killing activity directed to cells expressing a specific antigen. As is known in the art, lymphocyte response, in particular, T cell response is dependent on recognition of peptides by the T cell receptor, in particular, in the context of an MHC complex. Accordingly, the present invention provides for the culture of a lymphocyte population in the presence of peptides to which a desired response is to be directed. For example, the peptides can be known antigens associated with a disease and/or can be antigens determined in the subject to be treated, e.g. neoantigens as determined from analysis of a tumor sample or sample of infected tissue. The samples comprising the lymphocytes and/or the lymphocyte cultures may be exposed to between 2 and 300 peptides (whether as soluble peptides or as presented by antigen-presenting cells (APCs) as described herein).

The peptides to be included in the culture with the lymphocytes (or sample comprising lymphocytes) can be in soluble form. Where soluble peptides are used, they can be cultured with the lymphocytes at concentrations of 0.1 to 10 micromolar, 0.5 to 5 micromolar, or 1 to 2 micromolar. Alternatively or additionally, the peptides in the culture can be presented by APCs as is known in the art.

It is preferred herein that antigenic peptides are added to the culture such that they can be presented to lymphocytes by B cells in an MHC-dependent manner. Preferably, the peptides that are added to the lymphocytes have lengths between 9 and 35 amino acids, between 9 and 30, between 9 and 25. In certain embodiments, the antigenic peptides that are added to the lymphocytes are peptides that are presented by MHC class I molecules. Such peptides usually have a length of 9 to 12 amino acids. In certain embodiments, the antigenic peptides that are added to the lymphocytes are peptides that are presented by MHC class II molecules. Such peptides usually have a length of 13 to 25 amino acids. In certain embodiments, the antigenic peptides that are added to the lymphocytes may be a mix of peptides that are presented by MHC class I or MHC class II molecules. Such peptides may have a length of 9 to 25 amino acids. However, the peptides that are added to the culture may also be longer peptides that are taken up by an APC and processed into a shorter peptide that can be displayed in an MHC-dependent manner.

A non-limiting example of APCs of use in the methods herein includes B cells. B cells are known to stimulate the specific population of lymphocytes, in particular T cells (including TILs), responsive to the antigen presented. The APCs, e.g. B cells, may be either from an allogenic source (one or multiple apheresis from one or more donors) or autologous as described herein. The APCs may be retrieved from frozen or fresh aphereses according to methods known in the art. In the context of B cells, they may be selecting using a LOVO (Fresenius Kabi), Prodigy (Miltenyi biotec), EKKO (Millipore, Sigma) equipment or other cell separation technology. The APCs, in particular B cells, may be activated, e.g. using antibody CD 40 coated beads (Miltenyi Biotec and/or Adipogen). The autologous or allogenic APCs may be treated with mRNA to express the antigens as disclosed herein Additionally, the APCs may be cultured in the presence of nucleotide sequences containing the retrieved peptide sequences, the same transduction could be done with the TILs or T cells in culture.

The APCs, e.g. B cells, can be engineered to present the desired antigen by any means known in the art or described herein, e.g. coated with peptide or engineered by recombinant technology to express and process the antigens for presentation in the context of an MHC at the cell surface In a non-limiting example, the APCs may be either incubated and expanded for 0-4 days or immediately transfected and/or expanded for up to 4 day in static culture or in bioreactors prior to culture with the sample known or believed to containing the leucocytes. Bioreactors for culture of the APCs include but are not limited to ADVA (from ADVA Biotech); WAVE Bioreactor (Cytiva), GRex (Wilson Wolff), Ori Bioreactor (Ori), and Cocoon (Lonza). Alternatively, APCs may also be cultured in a gas permeable culture bag. In the context of B cells, quality may be assessed by testing for CD20+ cells. In particular embodiments, 85% or more of the cells in the B cell culture are CD20+.

In certain embodiments, B cells are prepared before they are added to the lymphocytes. Initially, B cells may be obtained from PBMCs by means of cell selection. PBMCs are preferably obtained by apheresis. When B cells (or any other type of APCs) are used in the preparation of a population of lymphocytes for autologous cell therapy, it is required that B cells are obtained from the same patient as the lymphocytes.

Kits for isolating B cells from PBMCs are known in the art and commercially available. The isolated B cells are preferably activated before adding them to the lymphocytes. Preferably, B cells are activated for 0-20 days, 0-15 days, 0-12 days, 0-10 days, 0-7 days, 0-5 days or 0-2 days. In certain embodiments, B cells may be activated for 1-48 hours, 8-48 hours or 12-36 hours. For example, activation of B cells may be achieved by contacting the B cells with IL-4 and/or CD40L. Further, B cells may be activated in the presence of IL-21.

Where the APCs are transfected to express the antigen of interest, it may be performed by any means known in the art, including but not limited to electroporation, PEG, lipofection or Crispr Cas. The APCs may alternately or additionally be transfected to express immunomodulators such as, e.g. OX40L, 4-1BBL, CD80, CD86, CD83, CD70, CD40L, GITR-L, CD127L, CD30L (CD153), LIGHT, BTLA, ICOS-L (CD275), SLAM (CD150), CD662L, interleukin-12, interleukin-7, interleukin-15, interleukin-17, interleukin-21, interleukin-4, Bcl6, Bcl-XL, BCL-2, MCL1, or STAT-5. Alternately or additionally, the APCs may be transfected with one or more activators of at least one signaling pathway such as the JAK/STAT pathway, the Akt/PBK AKT signaling pathway, the BCR signaling pathway, or the BAFF/BAFFR signaling pathway.

In a non-limiting example, the APC
may express human OX40L as set forth
in SEQ ID NO: 1:
MVSHRYPRIQSIKVQFTEYKKEKGFILTSQKEDEIMKVQNNSVII
NCDGFYLISLKGYFSQEVNISLHYQKDEEPLFOLKKVRSVNSLMV
ASLTYKDKVYLNVTTDNTSLDDFHVNGGELILIHQNPGEFCVL;
or as encoded by the DNA sequence as
set forth in SEQ ID NO: 2:
ATGGTATCACATCGGTATCCTCGAATTCAAAGTATCAAAGTACAA
TTTACCGAATATAAGAAGGAGAAAGGTTTCATCCTCACTTCCCAA
AAGGAGGATGAAATCATGAAGGTGCAGAACAACTCAGTCATCATC
AACTGTGATGGGTTTTATCTCATCTCCCTGAAGGGCTACTTCTCC
CAGGAAGTCAACATTAGCCTTCATTACCAGAAGGATGAGGAGCCC
CTCTTCCAACTGAAGAAGGTCAGGTCTGTCAACTCCTTGATGGTG
GCCTCTCTGACTTACAAAGACAAAGTCTACTTGAATGTGACCACT
GACAATACCTCCCTGGATGACTTCCATGTGAATGGCGGAGAACTG
ATTCTTATCCATCAAAATCCTGGTGAATTCTGTGTCCTTTGA.
In another non-limiting example, the APC
may express murine OX40L as set forth in
SEQ ID NO: 3:
MEGEGVQPLDENLENGSRPRFKWKKTLRLVVSGIKGAGMLLCFIY
VCLQLSSSPAKDPPIQRLRGAVTRCEDGOLFISSYKNEYQTMEVO
NNSVVIKCDGLYIIYLKGSFFQEVKIDHFREDHNPISIPMLNDGR
RIVFTVVASLAFKDKVYLTVNAPDTLCEHLQINDGELIVVOLTPG
YCAPEGSYHSTVNQVPL;
or as encoded by the DNA sequence as set
forth in SEQ ID NO: 4:
ATGGAAGGGGAAGGGGTTCAACCCCTGGATGAGAATCTGGAAAAC
GGATCAAGGCCAAGATTCAAGTGGAAGAAGACGCTAAGGCTGGTG
GTCTCTGGGATCAAGGGAGCAGGGATGCTTCTGTGCTTCATCTAT
GTCTGCCTGCAACTCTCTTCCTCTCCGGCAAAGGACCCTCCAATC
CAAAGACTCAGAGGAGCAGTTACCAGATGTGAGGATGGGCAACTA
TTCATCAGCTCATACAAGAATGAGTATCAAACTATGGAGGTGCAG
AACAATTCGGTTGTCATCAAGTGCGATGGGCTTTATATCATCTAC
CTGAAGGGCTCCTTTTTCCAGGAGGTCAAGATTGACCTTCATTTC
CGGGAGGATCATAATCCCATCTCTATTCCAATGCTGAACGATGGT
CGAAGGATTGTCTTCACTGTGGTGGCCTCTTTGGCTTTCAAAGAT
AAAGTTTACCTGACTGTAAATGCTCCTGATACTCTCTGCGAACAC
CTCCAGATAAATGATGGGGAGCTGATTGTTGTCCAGCTAACGCCT
GGATACTGTGCTCCTGAAGGATCTTACCACAGCACTGTGAACCAA
GTACCACTGTGA.
In another non-limiting example, the APC
may express human 4-1BBL as set forth in
SEQ ID NO: 5:
MEYASDASLDPEAPWPPAPRARACRVLPWALVAGLLLLLLLAAAC
AVFLACPWAVSGARASPGSAASPRLREGPELSPDDPAGLLDLRQG
MFAQLVAQNVLLIDGPLSWYSDPGLAGVSLTGGLSYKEDTKELVV
AKAGVYYVFFQLELRRVVAGEGSGSVSLALHLQPLRSAAGAAALA
LTVDLPPASSEARNSAFGFQGRLLHLSAGQRLGVHLHTEARARHA
WQLTQGATVLGLFRVTPEIPAGLPSPRSE;
or as encoded by the DNA sequence as set
forth in SEQ ID NO: 6:
ATGGAATACGCCTCTGACGCTTCACTGGACCCCGAAGCCCCGTGG
CCTCCCGCGCCCCGCGCTCGCGCCTGCCGCGTACTGCCTTGGGCC
CTGGTCGCGGGGCTGCTGCTGCTGCTGCTGCTCGCTGCCGCCTGC
GCCGTCTTCCTCGCCTGCCCCTGGGCCGTGTCCGGGGCTCGCGCC
TCGCCCGGCTCCGCGGCCAGCCCGAGACTCCGCGAGGGTCCCGAG
CTTTCGCCCGACGATCCCGCCGGCCTCTTGGACCTGCGGCAGGGC
ATGTTTGCGCAGCTGGTGGCCCAAAATGTTCTGCTGATCGATGGG
CCCCTGAGCTGGTACAGTGACCCAGGCCTGGCAGGCGTGTCCCTG
ACGGGGGGCCTGAGCTACAAAGAGGACACGAAGGAGCTGGTGGTG
GCCAAGGCTGGAGTCTACTATGTCTTCTTTCAACTAGAGCTGCGG
CGCGTGGTGGCCGGCGAGGGCTCAGGCTCCGTTTCACTTGCGCTG
CACCTGCAGCCACTGCGCTCTGCTGCTGGGGCCGCCGCCCTGGCT
TTGACCGTGGACCTGCCACCCGCCTCCTCCGAGGCTCGGAACTCG
GCCTTCGGTTTCCAGGGCCGCTTGCTGCACCTGAGTGCCGGCCAG
CGCCTGGGCGTCCATCTTCACACTGAGGCCAGGGCACGCCATGCC
TGGCAGCTTACCCAGGGCGCCACAGTCTTGGGACTCTTCCGGGTG
ACCCCCGAAATCCCAGCCGGACTCCCTTCACCGAGGTCGGAATAA.
In another non-limiting example, the APC
may express murine 4-1BBL as set forth in
SEQ ID NO: 7:
MDQHTLDVEDTADARHPAGTSCPSDAALLRDTGLLADAALLSDTV
RPTNAALPTDAAYPAVNVRDREAAWPPALNFCSRHPKLYGLVALV
LLLLIAACVPIFTRTEPRPALTITTSPNLGTRENNADQVTPVSHI
GCPNTTQQGSPVFAKLLAKNQASLCNTTLNWHSQDGAGSSYLSQG
LRYEEDKKELVVDSPGLYYVFLELKLSPTFTNTGHKVQGWVSLVL
QAKPQVDDFDNLALTVELFPCSMENKLVDRSWSQLLLLKAGHRLS
VGLRAYLHGAQDAYRDWELSYPNTTSFGLFLVKPDNPWE;
or as encoded by the DNA sequence as
set forth in SEQ ID NO: 8:
ATGGACCAGCACACACTTGATGTGGAGGATACCGCGGATGCCAGA
CATCCAGCAGGTACTTCGTGCCCCTCGGATGCGGCGCTCCTCAGA
GATACCGGGCTCCTCGCGGACGCTGCGCTCCTCTCAGATACTGTG
CGCCCCACAAATGCCGCGCTCCCCACGGATGCTGCCTACCCTGCG
GTTAATGTTCGGGATCGCGAGGCCGCGTGGCCGCCTGCACTGAAC
TTCTGTTCCCGCCACCCAAAGCTCTATGGCCTAGTCGCTTTGGTT
TTGCTGCTTCTGATCGCCGCCTGTGTTCCTATCTTCACCCGCACC
GAGCCTCGGCCAGCGCTCACAATCACCACCTCGCCCAACCTGGGT
ACCCGAGAGAATAATGCAGACCAGGTCACCCCTGTTTCCCACATT
GGCTGCCCCAACACTACACAACAGGGCTCTCCTGTGTTCGCCAAG
CTACTGGCTAAAAACCAAGCATCGTTGTGCAATACAACTCTGAAC
TGGCACAGCCAAGATGGAGCTGGGAGCTCATACCTATCTCAAGGT
CTGAGGTACGAAGAAGACAAAAAGGAGTTGGTGGTAGACAGTCCC
GGGCTCTACTACGTATTTTTGGAACTGAAGCTCAGTCCAACATTC
ACAAACACAGGCCACAAGGTGCAGGGCTGGGTCTCTCTTGTITTG
CAAGCAAAGCCTCAGGTAGATGACTTTGACAACTTGGCCCTGACA
GTGGAACTGTTCCCTTGCTCCATGGAGAACAAGTTAGTGGACCGT
TCCTGGAGTCAACTGTTGCTCCTGAAGGCTGGCCACCGCCTCAGT
GTGGGTCTGAGGGCTTATCTGCATGGAGCCCAGGATGCATACAGA
GACTGGGAGCTGTCTTATCCCAACACCACCAGCTTTGGACTCTTT
CTTGTGAAACCCGACAACCCATGGGAATGA.
In another non-limiting example, the APC
may express human CD80 as set forth in
SEQ ID NO: 9:
MEVPPPAPRSFLCRALCLFPRVFAAEAVTADSEVLEERQKRLPYV
PEPYYPESGWDRLRELFGKDEQQRISKDLANICKTAATAGIIGWV
YGGIPAFIHAKQQYIEQSQAEIYHNRFDAVQSAHRAATRGFIRYG
WRWGWRTAVFVTIFNTVNTSLNVYRNKDALSHFVIAGAVTGSLFR
INVGLRGLVAGGIIGALLGTPVGGLLMAFQKYSGETVQERKQKDR
KALHELKLEEWKGRLQVTEHLPEKIESSLQEDEPENDAKKIEALL
NLPRNPSVIDKQDKD;
or as encoded by the DNA sequence set
forth in SEQ ID NO: 10:
ATGGAGGTGCCGCCACCGGCACCGCGGAGCTTTCTCTGTAGAGCA
TTGTGCCTATTTCCCCGAGTCTTTGCTGCCGAAGCTGTGACTGCC
GATTCGGAAGTCCTTGAGGAGCGTCAGAAGCGGCTTCCCTACGTC
CCAGAGCCCTATTACCCGGAATCTGGATGGGACCGCCTCCGGGAG
CTGTITGGCAAAGATGAACAGCAGAGAATTTCAAAGGACCTTGCT
AATATCTGTAAGACGGCAGCTACAGCAGGCATCATTGGCTGGGTG
TATGGGGGAATACCAGCTTTTATTCATGCTAAACAACAATACATT
GAGCAGAGCCAGGCAGAAATTTATCATAACCGGTTTGATGCTGTG
CAATCTGCACATCGTGCTGCCACACGAGGCTTCATTCGTTATGGC
TGGCGCTGGGGTTGGAGAACTGCAGTGTTTGTGACTATATTCAAC
ACAGTGAACACTAGTCTGAATGTATACCGAAATAAAGATGCCTTA
AGCCATTTTGTAATTGCAGGAGCTGTCACGGGAAGTCTTTTTAGG
ATAAACGTAGGCCTGCGTGGCCTGGTGGCTGGTGGCATAATTGGA
GCCTTGCTGGGCACTCCTGTAGGAGGCCTGCTGATGGCATTTCAG
AAGTACTCTGGTGAGACTGTTCAGGAAAGAAAACAGAAGGATCGA
AAGGCACTCCATGAGCTAAAACTGGAAGAGTGGAAAGGCAGACTA
CAAGTTACTGAGCACCTCCCTGAGAAAATTGAAAGTAGTTTACAG
GAAGATGAACCTGAGAATGATGCTAAGAAAATTGAAGCACTGCTA
AACCTTCCTAGAAACCCTTCAGTAATAGATAAACAAGACAAGGAC
TGA.
In another non-limiting example, the APC
may express murine CD80 as set forth in
SEQ ID NO: 11:
MACNCQLMQDTPLLKFPCPRLILLFVLLIRLSQVSSDVDEQLSKS
VKDKVLLPCRYNSPHEDESEDRIYWQKHDKVVLSVIAGKLKVWPE
YKNRTLYDNTTYSLIILGLVLSDRGTYSCVVQKKERGTYEVKHLA
LVKLSIKADFSTPNITESGNPSADTKRITCFASGGFPKPRFSWLE
NGRELPGINTTISQDPESELYTISSQLDFNTTRNHTIKCLIKYGD
AHVSEDFTWEKPPEDPPDSKNTLVLFGAGFGAVITVVVIVVIIKC
FCKHRSCFRRNEASRETNNSLTFGPEEALAEQTVFL;
or as encoded by the DNA sequence set
forth in SEQ ID NO: 12:
ATGGCTTGCAATTGTCAGTTGATGCAGGATACACCACTCCTCAAG
TTTCCATGTCCAAGGCTCATTCTTCTCTTTGTGCTGCTGATTCGT
CTTTCACAAGTGTCTTCAGATGTTGATGAACAACTGTCCAAGTCA
GTGAAAGATAAGGTATTGCTGCCTTGCCGTTACAACTCTCCTCAT
GAAGATGAGTCTGAAGACCGAATCTACTGGCAAAAACATGACAAA
GTGGTGCTGTCTGTCATTGCTGGGAAACTAAAAGTGTGGCCCGAG
TATAAGAACCGGACTTTATATGACAACACTACCTACTCTCTTATC
ATCCTGGGCCTGGTCCTTTCAGACCGGGGCACATACAGCTGTGTC
GTTCAAAAGAAGGAAAGAGGAACGTATGAAGTTAAACACTTGGCT
TTAGTAAAGTTGTCCATCAAAGCTGACTTCTCTACCCCCAACATA
ACTGAGTCTGGAAACCCATCTGCAGACACTAAAAGGATTACCTGC
TTTGCTTCCGGGGGTTTCCCAAAGCCTCGCTTCTCTTGGTTGGAA
AATGGAAGAGAATTACCTGGCATCAATACGACAATTTCCCAGGAT
CCTGAATCTGAATTGTACACCATTAGTAGCCAACTAGATTTCAAT
ACGACTCGCAACCACACCATTAAGTGTCTCATTAAATATGGAGAT
GCTCACGTGTCAGAGGACTTCACCTGGGAAAAACCCCCAGAAGAC
CCTCCTGATAGCAAGAACACACTTGTGCTCTTTGGGGCAGGATTC
GGCGCAGTAATAACAGTCGTCGTCATCGTTGTCATCATCAAATGC
TTCTGTAAGCACAGAAGCTGTTTCAGAAGAAATGAGGCAAGCAGA
GAAACAAACAACAGCCTTACCTTCGGGCCTGAAGAAGCATTAGCT
GAACAGACCGTCTTCCTT.
In another non-limiting example, the APC
may express human CD86 as set forth in
SEQ ID NO: 13:
MGRTSFDSDSWTLRLHNLQIKDKGLYQCIIHHKKPTGMIRIHQMN
SELSVLANFSQPEIVPISNITENVYINLTCSSIHGYPEPKKMSVL
LRTKNSTIEYDGVMQKSQDNVTELYDVSISLSVSFPDVTSNMTIF
CILETDKTRLLSSPFSIELEDPQPPPDHIPWITAVLPTVIICVMV
FCLILWKWKKKKRPRNSYKCGTNTMEREESEQTKKREKIHIPERS
DEAQRVFKSSKTSSCDKSDTCF;
or as encoded by the DNA sequence set
forth in SEQ ID NO: 14:
ATGGGCCGCACAAGTTTTGATTCGGACAGTTGGACCCTGAGACTT
CACAATCTTCAGATCAAGGACAAGGGCTTGTATCAATGTATCATC
CATCACAAAAAGCCCACAGGAATGATTCGCATCCACCAGATGAAT
TCTGAACTGTCAGTGCTTGCTAACTTCAGTCAACCTGAAATAGTA
CCAATTTCTAATATAACAGAAAATGTGTACATAAATTTGACCTGC
TCATCTATACACGGTTACCCAGAACCTAAGAAGATGAGTGTTTTG
CTAAGAACCAAGAATTCAACTATCGAGTATGATGGTGTTATGCAG
AAATCTCAAGATAATGTCACAGAACTGTACGACGTTTCCATCAGC
TTGTCTGTTTCATTCCCTGATGTTACGAGCAATATGACCATCTTC
TGTATTCTGGAAACTGACAAGACGCGGCTTTTATCTTCACCTTTC
TCTATAGAGCTTGAGGACCCTCAGCCTCCCCCAGACCACATTCCT
TGGATTACAGCTGTACTTCCAACAGTTATTATATGTGTGATGGTT
TTCTGTCTAATTCTATGGAAATGGAAGAAGAAGAAGCGGCCTCGC
AACTCTTATAAATGTGGAACCAACACAATGGAGAGGGAAGAGAGT
GAACAGACCAAGAAAAGAGAAAAAATCCATATACCTGAAAGATCT
GATGAAGCCCAGCGTGTTTTTAAAAGTTCGAAGACATCTTCATGC
GACAAAAGTGATACATGTTTTTAA.
In another non-limiting example, the APC
may express murine CD86 as set forth in
SEQ ID NO: 15:
MDPRCTMGLAILIFVTVLLISDAVSVETQAYFNGTAYLPCPFTKA
QNISLSELVVFWQDQQKLVLYEHYLGTEKLDSVNAKYLGRTSFDR
NNWTLRLHNVQIKDMGSYDCFIQKKPPTGSIILQQTLTELSVIAN
FSEPEIKLAQNVTGNSGINLTCTSKQGHPKPKKMYFLITNSTNEY
GDNMQISQDNVTELFSISNSLSLSFPDGVWHMTVVCVLETESMKI
SSKPLNFTQEFPSPQTYWKEITASVTVALLLVMLLIIVCHKKPNQ
PSRPSNTASKLERDSNADRETINLKELEPQIASAKPNAE;
or as encoded by the DNA sequence set
forth in SEQ ID NO: 16:
ATGGACCCCAGATGCACCATGGGCTTGGCAATCCTTATCTTTGTG
ACAGTCTTGCTGATCTCAGATGCTGTTTCCGTGGAGACGCAAGCT
TATTTCAATGGGACTGCATATCTGCCGTGCCCATTTACAAAGGCT
CAAAACATAAGCCTGAGTGAGCTGGTAGTATTTTGGCAGGACCAG
CAAAAGTTGGTTCTGTACGAGCACTATTTGGGCACAGAGAAACTT
GATAGTGTGAATGCCAAGTACCTGGGCCGCACGAGCTTTGACAGG
AACAACTGGACTCTACGACTTCACAATGTTCAGATCAAGGACATG
GGCTCGTATGATTGTTTTATACAAAAAAAGCCACCCACAGGATCA
ATTATCCTCCAACAGACATTAACAGAACTGTCAGTGATCGCCAAC
TTCAGTGAACCTGAAATAAAACTGGCTCAGAATGTAACAGGAAAT
TCTGGCATAAATTTGACCTGCACGTCTAAGCAAGGTCACCCGAAA
CCTAAGAAGATGTATTTTCTGATAACTAATTCAACTAATGAGTAT
GGTGATAACATGCAGATATCACAAGATAATGTCACAGAACTGTTC
AGTATCTCCAACAGCCTCTCTCTTTCATTCCCGGATGGTGTGTGG
CATATGACCGTTGTGTGTGTTCTGGAAACGGAGTCAATGAAGATT
TCCTCCAAACCTCTCAATTTCACTCAAGAGTTTCCATCTCCTCAA
ACGTATTGGAAGGAGATTACAGCTTCAGTTACTGTGGCCCTCCTC
CTTGTGATGCTGCTCATCATTGTATGTCACAAGAAGCCGAATCAG
CCTAGCAGGCCCAGCAACACAGCCTCTAAGTTAGAGCGGGATAGT
AACGCTGACAGAGAGACTATCAACCTGAAGGAACTTGAACCCCAA
ATTGCTTCAGCAAAACCAAATGCAGAGTGA.
In another non-limiting example, the APC
may express human CD83 as set forth in
SEQ ID NO: 17:
METPQEDHLRGQHYHQKGQNGSFDAPNERPYSLKIRNTTSCNSGT
YRCTLQDPDGQRNLSGKVILRVTGCPAQRKEETFKKYRAEIVLLL
ALVIFYLTLIIFTCKFARLQSIFPDFSKAGMERAFLPVTSPNKHL
GLVTPHKTELV;
or as encoded by the DNA sequence set forth in
SEQ ID NO: 18:
ATGGAGACACCCCAGGAAGACCACCTCAGGGGACAGCACTATCAT
CAGAAGGGGCAAAATGGTTCTTTCGACGCCCCCAATGAAAGGCCC
TATTCCCTGAAGATCCGAAACACTACCAGCTGCAACTCGGGGACA
TACAGGTGCACTCTGCAGGACCCGGATGGGCAGAGAAACCTAAGT
GGCAAGGTGATCTTGAGAGTGACAGGATGCCCTGCACAGCGTAAA
GAAGAGACTTTTAAGAAATACAGAGCGGAGATTGTCCTGCTGCTG
GCTCTGGTTATTTTCTACTTAACACTCATCATTTTCACTTGTAAG
TTTGCACGGCTACAGAGTATCTTCCCAGATTTTTCTAAAGCTGGC
ATGGAACGAGCTTTTCTCCCAGTTACCTCCCCAAATAAGCATTTA
GGGCTAGTGACTCCTCACAAGACAGAACTGGTATGA.
In another non-limiting example, the APC
may express murine CD83 as set forth in
SEQ ID NO: 19:
MSQGLQLLFLGCACSLAPAMAMREVTVACSETADLPCTAPWDPQL
SYAVSWAKVSESGTESVELPESKQNSSFEAPRRRAYSLTIQNTTI
CSSGTYRCALQELGGQRNLSGTVVLKVTGCPKEATESTFRKYRAE
AVLLFSLVVFYLTLIIFTCKFARLOSIFPDISKPGTEQAFLPVTS
PSKHLGPVTLPKTETV;
or as encoded by the DNA sequence set forth in
SEQ ID NO: 20:
ATGTCGCAAGGCCTCCAGCTCCTGTTTCTAGGCTGCGCCTGCAGC
CTGGCACCCGCGATGGCGATGCGGGAGGTGACGGTGGCTTGCTCC
GAGACCGCCGACTTGCCTTGCACAGCGCCCTGGGACCCGCAGCTC
TCCTATGCAGTGTCCTGGGCCAAGGTCTCCGAGAGTGGCACTGAG
AGTGTGGAGCTCCCGGAGAGCAAGCAAAACAGCTCCTTCGAGGCC
CCCAGGAGAAGGGCCTATTCCCTGACGATCCAAAACACTACCATC
TGCAGCTCGGGCACCTACAGGTGTGCCCTGCAGGAGCTCGGAGGG
CAGCGCAACTTGAGCGGCACCGTGGTTCTGAAGGTGACAGGATGC
CCCAAGGAAGCTACAGAGTCAACTTTCAGGAAGTACAGGGCAGAA
GCTGTGTTGCTCTTCTCTCTGGTTGTTTTCTACCTGACACTCATC
ATTTTCACCTGCAAATTTGCACGACTACAAAGCATTTTCCCAGAT
ATTTCTAAACCTGGTACGGAACAAGCTTTTCTTCCAGTCACCTCC
CCAAGCAAACATTTGGGGCCAGTGACCCTTCCTAAGACAGAAACG
GTATGA.
In another non-limiting example, the APC
may express human CD70 as set forth in
SEQ ID NO: 21:
MPEEGSGCSVRRRPYGCVLRAALVPLVAGLVICLVVCIQRFAQAQ
QQLPLESLGWDVAELQLNHTGPQQDPRLYWQGGPALGRSFLHGPE
LDKGQLRIHRDGIYMVHIQVTLAICSSTTASRHHPTTLAVGICSP
ASRSISLLRLSFHQGLFGFWNWGLKVKCFLRHLIWTAHCFIPLTQ
LVFMQALQSWRNHHCSHFTDEENRGVNR;
or as encoded by the DNA sequence set forth
in SEQ ID NO: 22:
ATGCCGGAGGAGGGTTCGGGCTGCTCGGTGCGGCGCAGGCCCTAT
GGGTGCGTCCTGCGGGCTGCTTTGGTCCCATTGGTCGCGGGCTTG
GTGATCTGCCTCGTGGTGTGCATCCAGCGCTTCGCACAGGCTCAG
CAGCAGCTGCCGCTCGAGTCACTTGGGTGGGACGTAGCTGAGCTG
CAGCTGAATCACACAGGACCTCAGCAGGACCCCAGGCTATACTGG
CAGGGGGGCCCAGCACTGGGCCGCTCCTTCCTGCATGGACCAGAG
CTGGACAAGGGGCAGCTACGTATCCATCGTGATGGCATCTACATG
GTACACATCCAGGTGACGCTGGCCATCTGCTCCTCCACGACGGCC
TCCAGGCACCACCCCACCACCCTGGCCGTGGGAATCTGCTCTCCC
GCCTCCCGTAGCATCAGCCTGCTGCGTCTCAGCTTCCACCAAGGG
CTTTTTGGATTTTGGAACTGGGGACTCAAAGTCAAGTGCTTCTTA
CGGCATTTAATATGGACTGCACACTGTITTATCCCATTAACTCAG
CTCGTGTTCATGCAAGCCCTACAAAGCTGGAGGAATCATCATTGT
TCCCATTTCACAGATGAGGAAAACAGAGGCGTAAACCGTTGA.
In another non-limiting example, the APC
may express murine CD70 as set forth in
SEQ ID NO: 23:
MPEEGRPCPWVRWSGTAFQRQWPWLLLVVFITVFCCWFHCSGLLS
KQQQRLLEHPEPHTAELQLNLTVPRKDPTLRWGAGPALGRSFTHG
PELEEGHLRIHQDGLYRLHIQVTLANCSSPGSTLQHRATLAVGIC
SPAAHGISLLRGRFGQDCTVALQRLTYLVHGDVLCTNLTLPLLPS
RNADETFFGVQWICP;
or as encoded by the DNA sequence set forth in
SEQ ID NO: 24:
ATGCCGGAGGAAGGTCGCCCTTGCCCCTGGGTTCGCTGGAGCGGG
ACCGCGTTCCAGCGCCAATGGCCATGGCTGCTGCTGGTGGTGTTT
ATTACTGTGTTTTGCTGTTGGTTTCATTGTAGCGGACTACTCAGT
AAGCAGCAACAGAGGCTGCTGGAGCACCCTGAGCCGCACACAGCT
GAGTTACAGCTGAATCTCACAGTTCCTCGGAAGGACCCCACACTG
CGCTGGGGAGCAGGCCCAGCCTTGGGAAGGTCCTTCACACACGGA
CCAGAGCTGGAGGAGGGCCATCTGCGTATCCATCAAGATGGCCTC
TACAGGCTGCATATCCAGGTGACACTGGCCAACTGCTCTTCCCCA
GGCAGCACCCTGCAGCACAGGGCCACCCTGGCTGTGGGCATCTGC
TCCCCCGCTGCGCACGGCATCAGCTTGCTGCGTGGGCGCTTTGGA
CAGGACTGTACAGTGGCATTACAGCGCCTGACATACCTGGTCCAC
GGAGATGTCCTCTGTACCAACCTCACCCTGCCTCTGCTGCCGTCC
CGCAACGCTGATGAGACCTTCTTTGGAGTTCAGTGGATATGCCCT
TGA.
In another non-limiting example, the APC
may express human IL7/CD127 as set forth in
SEQ ID NO: 25:
MTILGTTFGMVFSLLQVVSGESGYAQNGDLEDAELDDYSFSCYSQ
LEVNGSQHSLTCAFEDPDVNITNLEFEICGALVEVKCLNFRKLQE
IYFIETKKFLLIGKSNICVKVGEKSLTCKKIDLTTIVKPEAPFDL
SVVYREGANDFVVTFNTSHLQKKYVKVLMHDVAYRQEKDENKWTH
VNLSSTKLTLLORKLQPAAMYEIKVRSIPDHYFKGFWSEWSPSYY
FRTPEINNSSGLSLSYGPVSPIIRRLWNIFVRNQEK;
or as encoded by the DNA sequence set forth
in SEQ ID NO: 26:
ATGACAATTCTAGGTACAACTTTTGGCATGGTTTTTTCTTTACTT
CAAGTCGTTTCTGGAGAAAGTGGCTATGCTCAAAATGGAGACTTG
GAAGATGCAGAACTGGATGACTACTCATTCTCATGCTATAGCCAG
TTGGAAGTGAATGGATCGCAGCACTCACTGACCTGTGCTTTTGAG
GACCCAGATGTCAACATCACCAATCTGGAATTTGAAATATGTGGG
GCCCTCGTGGAGGTAAAGTGCCTGAATTTCAGGAAACTACAAGAG
ATATATTTCATCGAGACAAAGAAATTCTTACTGATTGGAAAGAGC
AATATATGTGTGAAGGTTGGAGAAAAGAGTCTAACCTGCAAAAAA
ATAGACCTAACCACTATAGTTAAACCTGAGGCTCCTTTTGACCTG
AGTGTCGTCTATCGGGAAGGAGCCAATGACTTTGTGGTGACATTT
AATACATCACACTTGCAAAAGAAGTATGTAAAAGTITTAATGCAC
GATGTAGCTTACCGCCAGGAAAAGGATGAAAACAAATGGACGCAT
GTGAATTTATCCAGCACAAAGCTGACACTCCTGCAGAGAAAGCTC
CAACCGGCAGCAATGTATGAGATTAAAGTTCGATCCATCCCTGAT
CACTATTTTAAAGGCTTCTGGAGTGAATGGAGTCCAAGTTATTAC
TTCAGAACTCCAGAGATCAATAATAGCTCAGGATTAAGCCTATCG
TATGGCCCAGTCTCCCCGATCATAAGAAGACTCTGGAACATCTTT
GTAAGAAACCAAGAAAAGTGA.
In another non-limiting example, the APC
may express murine IL7/CD127 as set forth
in SEQ ID NO: 27:
MMALGRAFAIVFCLIQAVSGESGNAQDGDLEDADADDHSFWCHSQ
LEVDGSQHLLTCAFNDSDINTANLEFQICGALLRVKCLTLNKLQD
IYFIKTSEFLLIGSSNICVKLGQKNLTCKNMAINTIVKAEAPSDL
KVVYRKEANDFLVTFNAPHLKKKYLKKVKHDVAYRPARGESNWTH
VSLFHTRTTIPQRKLRPKAMYEIKVRSIPHNDYFKGFWSEWSPSS
TFETPEPKNQGGWDPVLPSVTILSLFSVFLLVILAHVLWKKRIKP
VVWPSLPDHKKTLEQL;
or as encoded by the DNA sequence set forth
in SEQ ID NO: 28:
ATGATGGCTCTGGGTAGAGCTTTCGCTATAGTTTTCTGCTTAATT
CAAGCTGTTTCTGGAGAAAGTGGAAATGCCCAGGATGGAGACCTA
GAAGATGCAGACGCGGACGATCACTCCTTCTGGTGCCACAGCCAG
TTGGAAGTGGATGGAAGTCAACATTTATTGACTTGTGCTTTTAAT
GACTCAGACATCAACACAGCTAATCTGGAATTTCAAATATGTGGG
GCTCTTTTACGAGTGAAATGCCTAACTCTTAACAAGCTGCAAGAT
ATATATTTTATAAAGACATCAGAATTCTTACTGATTGGTAGCAGC
AATATATGTGTGAAGCTTGGACAAAAGAATTTAACTTGCAAAAAT
ATGGCTATAAACACAATAGTTAAAGCCGAGGCTCCCTCTGACCTG
AAAGTCGTTTATCGCAAAGAAGCAAATGATTTTTTGGTGACATTT
AATGCACCTCACTTGAAAAAGAAATATTTAAAAAAAGTAAAGCAT
GATGTGGCCTACCGCCCAGCAAGGGGTGAAAGCAACTGGACGCAT
GTATCTTTATTCCACACAAGAACAACAATCCCACAGAGAAAACTA
CGACCAAAAGCAATGTATGAAATCAAAGTCCGATCCATTCCCCAT
AACGATTACTTCAAAGGCTTCTGGAGCGAGTGGAGTCCAAGTTCT
ACCTTCGAAACTCCAGAACCCAAGAATCAAGGAGGATGGGATCCT
GTCTTGCCAAGTGTCACCATTCTGAGTTTGTTCTCTGTGTTTTTG
TTGGTCATCTTAGCCCATGTGCTATGGAAAAAAAGGATTAAACCT
GTCGTATGGCCTAGTCTCCCCGATCATAAGAAAACTCTGGAACAA
CTATAG.
In another non-limiting example, the APC
may express human CD30L as set forth in
SEQ ID NO: 29:
MDPGLQQALNGMAPPGDTAMHVPAGSVASHLGTTSRSYFYLTTAT
LALCLVFTVATIMVLVVQRTDSIPNSPDNVPLKGGNCSEDLLCIL
KRAPFKKSWAYLQVAKHLNKTKLSWNKDGILHGVRYQDGNLVIQF
PDYCGMILHHSHSTLDSGKGHCCLETLQP;
or as encoded by the DNA sequence set
forth in SEQ ID NO: 30:
ATGGACCCAGGGCTGCAGCAAGCACTCAACGGAATGGCCCCTCCT
GGAGACACAGCCATGCATGTGCCGGCGGGCTCCGTGGCCAGCCAC
CTGGGGACCACGAGCCGCAGCTATTTCTATTTGACCACAGCCACT
CTGGCTCTGTGCCTTGTCTTCACGGTGGCCACTATTATGGTGTTG
GTCGTTCAGAGGACGGACTCCATTCCCAACTCACCTGACAACGTC
CCCCTCAAAGGAGGAAATTGCTCAGAAGACCTCTTATGTATCCTG
AAAAGGGCTCCATTCAAGAAGTCATGGGCCTACCTCCAAGTGGCA
AAGCATCTAAACAAAACCAAGTTGTCTTGGAACAAAGATGGCATT
CTCCATGGAGTCAGATATCAGGATGGGAATCTGGTGATCCAATTC
CCTGATTACTGTGGCATGATCCTCCACCATTCACACTCTACCCTG
GACTCTGGGAAGGGACACTGCTGCCTTGAAACTCTACAACCCTGA
In another non-limiting example, the APC
may express murine CD30L as set forth in
SEQ ID NO: 31:
MEPGLQQAGSCGAPSPDPAMQVQPGSVASPWRSTRPWRSTSRSYF
YLSTTALVCLVVAVAIILVLVVQKKDSTPNTTEKAPLKGGNCSED
LFCTLKSTPSKKSWAYLQVSKHLNNTKLSWNEDGTIHGLIYQDGN
LIVQFPGLYFIVCQLQFLVQCSNHSVDLTLQLLINSKIKKQTLVT
VCESGVQSKNIYQNLSQFLLHYLQVNSTISVRVDNFQYVD;
or as encoded by the DNA sequence set forth
in SEQ ID NO: 32:
ATGGAGCCAGGGCTGCAACAAGCAGGCAGCTGTGGGGCTCCTTCC
CCTGACCCAGCCATGCAGGTGCAGCCCGGCTCGGTAGCCAGCCCC
TGGAGAAGCACGAGGCCCTGGAGAAGCACAAGTCGCAGCTACTTC
TACCTCAGCACCACCGCACTGGTGTGCCTTGTTGTGGCAGTGGCG
ATCATTCTGGTACTGGTAGTCCAGAAAAAGGACTCCACTCCAAAT
ACAACTGAGAAGGCCCCCCTTAAAGGAGGAAATTGCTCAGAGGAT
CTCTTCTGTACCCTGAAAAGTACTCCATCCAAGAAGTCATGGGCC
TACCTCCAAGTGTCAAAGCATCTCAACAATACCAAACTGTCATGG
AACGAAGATGGCACCATCCACGGACTCATATACCAGGACGGGAAC
CTGATAGTCCAATTCCCTGGCTTGTACTTCATCGTTTGCCAACTG
CAGTTCCTCGTGCAGTGCTCAAATCATTCTGTGGACCTGACATTG
CAGCTCCTCATCAATTCCAAGATCAAAAAGCAGACGTTGGTAACA
GTGTGTGAGTCTGGAGTTCAGAGTAAGAACATCTACCAGAATCTC
TCTCAGTTTTTGCTGCATTACTTACAGGTCAACTCTACCATATCA
GTCAGGGTGGATAATTTCCAGTATGTGGATACAAACACTTTCCCT
CTTGATAATGTGCTATCCGTCTTCTTATATAGTAGCTCAGACTGA.
In another non-limiting example, the APC
may express human LIGHT as set forth in
SEQ ID NO: 33:
MEPPGDWGPPPWRSTPKTDVLRLVLYLTFLGAPCYAPALPSCKED
EYPVGSECCPKCSPGYRVKEACGELTGTVCEPCPPGTYIAHLNGL
SKCLQCQMCDPAMGLRASRNCSRTENAVCGCSPGHFCIVQDGDHC
AACRAYATSSPGQRVQKGGTESQDTLCQNCPPGTFSPNGTLEECQ
HQTKCSWLVTKAGAGTSSSHWVWWFLSGSLVIVIVCSTVGLIICV
KRRKPR;
or as encoded by the DNA sequence set
forth in SEQ ID NO: 34:
ATGGAGCCTCCTGGAGACTGGGGGCCTCCTCCCTGGAGATCCACC
CCCAAAACCGACGTCTTGAGGCTGGTGCTGTATCTCACCTTCCTG
GGAGCCCCCTGCTACGCCCCAGCTCTGCCGTCCTGCAAGGAGGAC
GAGTACCCAGTGGGCTCCGAGTGCTGCCCCAAGTGCAGTCCAGGT
TATCGTGTGAAGGAGGCCTGCGGGGAGCTGACGGGCACAGTGTGT
GAACCCTGCCCTCCAGGCACCTACATTGCCCACCTCAATGGCCTA
AGCAAGTGTCTGCAGTGCCAAATGTGTGACCCAGCCATGGGCCTG
CGCGCGAGCCGGAACTGCTCCAGGACAGAGAACGCCGTGTGTGGC
TGCAGCCCAGGCCACTTCTGCATCGTCCAGGACGGGGACCACTGC
GCCGCGTGCCGCGCTTACGCCACCTCCAGCCCGGGCCAGAGGGTG
CAGAAGGGAGGCACCGAGAGTCAGGACACCCTGTGTCAGAACTGC
CCCCCGGGGACCTTCTCTCCCAATGGGACCCTGGAGGAATGTCAG
CACCAGACCAAGTGCAGCTGGCTGGTGACGAAGGCCGGAGCTGGG
ACCAGCAGCTCCCACTGGGTATGGTGGTTTCTCTCAGGGAGCCTC
GTCATCGTCATTGTTTGCTCCACAGTTGGCCTAATCATATGTGTG
AAAAGAAGAAAGCCAAGGG.
In another non-limiting example, the APC
may express murine LIGHT as set forth in
SEQ ID NO: 35:
MESVVQPSVFVVDGQTDIPFRRLEQNHRRRRCGTVQVSLALVLLL
GAGLATQGWFLLRLHQRLGDIVAHLPDGGKGSWEKLIQDQRSHQA
NPAAHLTGANASLIGIGGPLLWETRLGLAFLRGLTYHDGALVTME
PGYYYVYSKVQLSGVGCPQGLANGLPITHGLYKRTSRYPKELELL
VSRRSPCGRANSSRVWWDSSFLGGVVHLEAGEEVVVRVPGNRLVR
PRDGTRSYFGAFMV;
or as encoded by the DNA sequence set forth
in SEQ ID NO: 36:
ATGGAGAGTGTGGTACAGCCTTCAGTGTTTGTGGTGGATGGACAG
ACGGACATCCCATTCAGGCGGCTGGAACAGAACCACCGGAGACGG
CGCTGTGGCACTGTCCAGGTCAGCCTGGCCCTGGTGCTGCTGCTA
GGTGCTGGGCTGGCCACTCAGGGCTGGTTTCTCCTGAGACTGCAT
CAACGTCTTGGAGACATAGTAGCTCATCTGCCAGATGGAGGCAAA
GGCTCCTGGGAGAAGCTGATACAAGATCAACGATCTCACCAGGCC
AACCCAGCAGCACATCTTACAGGAGCCAACGCCAGCTTGATAGGT
ATTGGTGGACCTCTGTTATGGGAGACACGACTTGGCCTGGCCTTC
TTGAGGGGCTTGACGTATCATGATGGGGCCCTGGTGACCATGGAG
CCCGGTTACTACTATGTGTACTCCAAAGTGCAGCTGAGCGGCGTG
GGCTGCCCCCAGGGGCTGGCCAATGGCCTCCCCATCACCCATGGA
CTATACAAGCGCACATCCCGCTACCCGAAGGAGTTAGAACTGCTG
GTCAGTCGGCGGTCACCCTGTGGCCGGGCCAACAGCTCCCGAGTC
TGGTGGGACAGCAGCTTCCTGGGCGGCGTGGTACATCTGGAGGCT
GGGGAAGAGGTGGTGGTCCGCGTGCCTGGAAACCGCCTGGTCAGA
CCACGTGACGGCACCAGGTCCTATTTCGGAGCTTTCATGGTCTGA.
In another non-limiting example, the APC
may express human BTLA as set forth in
SEQ ID NO: 37:
MKTLPAMLGTGKLFWVFFLIPYLDIWNIHGKESCDVQLYIKRQSE
HSILAGDPFELECPVKYCANRPHVTWCKLNGTTCVKLEDRQTSWK
EEKNISFFILHFEPVLPNDNGSYRCSANFQSNLIESHSTTLYVTD
VKSASERPSKDEMASRPWLLYRLLPLGGLPLLITTCFCLFCCLRR
HQGKQNELSDTAGREINLVDAHLKSEQTEASTRQNSQVLLSETGI
YDNDPDLCFRMQEGSEVYSNPCLEENKPGIVYASLNHSVIGPNSR
LARNVKEAPTEYASICVRS;
or as encoded by the DNA sequence set
forth in SEQ ID NO: 38:
ATGAAGACATTGCCTGCCATGCTTGGAACTGGGAAATTATTTTGG
GTCTTCTTCTTAATCCCATATCTGGACATCTGGAACATCCATGGG
AAAGAATCATGTGATGTACAGCTTTATATAAAGAGACAATCTGAA
CACTCCATCTTAGCAGGAGATCCCTTTGAACTAGAATGCCCTGTG
AAATACTGTGCTAACAGGCCTCATGTGACTTGGTGCAAGCTCAAT
GGAACAACATGTGTAAAACTTGAAGATAGACAAACAAGTTGGAAG
GAAGAGAAGAACATTTCATTTTTCATTCTACATTTTGAACCAGTG
CTTCCTAATGACAATGGGTCATACCGCTGTTCTGCAAATTTTCAG
TCTAATCTCATTGAAAGCCACTCAACAACTCTTTATGTGACAGAT
GTAAAAAGTGCCTCAGAACGACCCTCCAAGGACGAAATGGCAAGC
AGACCCTGGCTCCTGTATCGTTTACTTCCTTTGGGGGGATTGCCT
CTACTCATCACTACCTGTTTCTGCCTGTTCTGCTGCCTGAGAAGG
CACCAAGGAAAGCAAAATGAACTCTCTGACACAGCAGGAAGGGAA
ATTAACCTGGTTGATGCTCACCTTAAGAGTGAGCAAACAGAAGCA
AGCACCAGGCAAAATTCCCAAGTACTGCTATCAGAAACTGGAATT
TATGATAATGACCCTGACCTTTGTTTCAGGATGCAGGAAGGGTCT
GAAGTTTATTCTAATCCATGCCTGGAAGAAAACAAACCAGGCATT
GTTTATGCTTCCCTGAACCATTCTGTCATTGGACCGAACTCAAGA
CTGGCAAGAAATGTAAAAGAAGCACCAACAGAATATGCATCCATA
TGTGTGAGGAGTTAA.
In another non-limiting example, the APC
may express murine BTLA as set forth
in SEQ ID NO: 39:
MKTVPAMLGTPRLFREFFILHLGLWSILCEKATKRNDEECPVOLT
ITRNSKQSARTGELFKIQCPVKYCVHRPNVTWCKHNGTICVPLEV
SPQLYTSWEENQSVPVFVLHFKPIHLSDNGSYSCSTNFNSQVINS
HSVTIHVTERTQNSSEHPLIISDIPDATNASGPSTMEERPGRTWL
LYTLLPLGALLLLLACVCLLCFLKRIQGKEKKPSDLAGRDTNLVD
IPASSRTNHQALPSGTGIYDNDPWSSMQDESELTISLQSERNNQG
IVYASLNHCVIGRNPRQENNMQEAPTEYASICVRS;
or as encoded by the DNA sequence set
forth in SEQ ID NO: 40:
ATGAAGACAGTGCCTGCCATGCTTGGGACTCCTCGGTTATTTAGG
GAATTCTTCATCCTCCATCTGGGCCTCTGGAGCATCCTTTGTGAG
AAAGCTACTAAGAGGAATGATGAAGAGTGTCCAGTGCAACTTACT
ATTACGAGGAATTCCAAACAGTCTGCCAGGACAGGAGAGTTATTT
AAAATTCAATGTCCTGTGAAATACTGTGTTCATAGACCTAATGTG
ACTTGGTGTAAGCACAATGGAACAATCTGTGTACCCCTTGAGGTT
AGCCCTCAGCTATACACTAGTTGGGAAGAAAATCAATCAGTTCCG
GTTTTTGTTCTCCACTTTAAACCAATACATCTCAGTGATAATGGG
TCGTATAGCTGTTCTACAAACTTCAATTCTCAAGTTATTAATAGC
CATTCAGTAACCATCCATGTGACAGAAAGGACTCAAAACTCTTCA
GAACACCCACTAATAATATCTGACATCCCAGATGCCACCAATGCC
TCAGGACCATCCACCATGGAAGAGAGGCCAGGCAGGACTTGGCTG
CTTTACACCTTGCTTCCTTTGGGGGCATTGCTTCTGCTCCTTGCC
TGTGTCTGCCTGCTCTGCTTTCTGAAAAGGATCCAAGGGAAAGAA
AAGAAGCCTTCTGACTTGGCAGGAAGGGACACTAACCTGGTTGAT
ATTCCAGCCAGTTCCAGGACAAATCACCAAGCACTGCCATCAGGA
ACTGGAATTTATGATAATGATCCCTGGTCTAGCATGCAGGATGAA
TCTGAATTGACAATTAGCTTGCAATCAGAGAGAAACAACCAGGGC
ATTGTTTATGCTTCTTTGAACCATTGTGTTATTGGAAGGAATCCA
AGACAGGAAAACAACATGCAGGAGGCACCCACAGAATATGCATCC
ATTTGTGTGAGAAGTTAA.
In another non-limiting example, the APC
may express human ICOS-L as set forth
in SEQ ID NO: 41:
MRLGSPGLLFLLFSSLRADTQEKEVRAMVGSDVELSCACPEGSRF
DLNDVYVYWQTSESKTVVTYHIPQNSSLENVDSRYRNRALMSPAG
MLRGDFSLRLFNVTPQDEQKFHCLVLSQSLGFQEVLSVEVTLHVA
ANFSVPVVSAPHSPSQDELTFTCTSINGYPRPNVYWINKTDNSLL
DQALQNDTVFLNMRGLYDVVSVLRIARTPSVNIGCCIENVLLQQN
LTVGSQTGNDIGERDKITENPVSTGEKNAATWSILAVLCLLVVVA
VAIGWVCRDRCLQHSYAGAWAVSPETELTESWNLLLLLS;
or as encoded by the DNA sequence set
forth in SEQ ID NO: 42:
ATGCGGCTGGGCAGTCCTGGACTGCTCTTCCTGCTCTTCAGCAGC
CTTCGAGCTGATACTCAGGAGAAGGAAGTCAGAGCGATGGTAGGC
AGCGACGTGGAGCTCAGCTGCGCTTGCCCTGAAGGAAGCCGTTTT
GATTTAAATGATGTTTACGTATATTGGCAAACCAGTGAGTCGAAA
ACCGTGGTGACCTACCACATCCCACAGAACAGCTCCTTGGAAAAC
GTGGACAGCCGCTACCGGAACCGAGCCCTGATGTCACCGGCCGGC
ATGCTGCGGGGCGACTTCTCCCTGCGCTTGTTCAACGTCACCCCC
CAGGACGAGCAGAAGTTTCACTGCCTGGTGTTGAGCCAATCCCTG
GGATTCCAGGAGGTTTTGAGCGTTGAGGTTACACTGCATGTGGCA
GCAAACTTCAGCGTGCCCGTCGTCAGCGCCCCCCACAGCCCCTCC
CAGGATGAGCTCACCTTCACGTGTACATCCATAAACGGCTACCCC
AGGCCCAACGTGTACTGGATCAATAAGACGGACAACAGCCTGCTG
GACCAGGCTCTGCAGAATGACACCGTCTTCTTGAACATGCGGGGC
TTGTATGACGTGGTCAGCGTGCTGAGGATCGCACGGACCCCCAGC
GTGAACATTGGCTGCTGCATAGAGAACGTGCTTCTGCAGCAGAAC
CTGACTGTCGGCAGCCAGACAGGAAATGACATCGGAGAGAGAGAC
AAGATCACAGAGAATCCAGTCAGTACCGGCGAGAAAAACGCGGCC
ACGTGGAGCATCCTGGCTGTCCTGTGCCTGCTTGTGGTCGTGGCG
GTGGCCATAGGCTGGGTGTGCAGGGACCGATGCCTCCAACACAGC
TATGCAGGTGCCTGGGCTGTGAGTCCGGAGACAGAGCTCACTGAA
TCCTGGAACCTGCTCCTTCTGCTCTCGTGA.
In another non-limiting example, the APC
may express murine ICOS-L as set forth
in SEQ ID NO: 43:
CPCFVSLGTRQPVWKKLHVSSGFFSGLGLFLLLLSSLCAASAETE
VGAMVGSNVVLSCIDPHRRHFNLSGLYVYWQIENPEVSVTYYLPY
KSPGINVDSSYKNRGHLSLDSMKQGNFSLYLKNVTPQDTQEFTCR
VFMNTATELVKILEEVVRLRVAANFSTPVISTSDSSNPGQERTYT
CMSKNGYPEPNLYWINTTDNSLIDTALQNNTVYLNKLGLYDVIST
LRLPWTSRGDVLCCVENVALHQNITSISQAESFTGNNTKNPQETH
NNELKVLVPVLAVLAAAAFVSFIIYRRTRPHRSYTGPKTVQLELT
DTWAPVPYQDYLIPRYLMSPCLKTRGLP;
or as encoded by the DNA sequence set
forth in SEQ ID NO: 44:
GTGTCCCTGTTTTGTGTCCTTGGGAACCAGGCAGCCTGTTTGGAA
GAAGCTCCATGTTTCTAGCGGGTTCTTTTCTGGTCTTGGTCTGTT
CTTGCTGCTGTTGAGCAGCCTCTGTGCTGCCTCTGCAGAGACTGA
AGTCGGTGCAATGGTGGGCAGCAATGTGGTGCTCAGCTGCATTGA
CCCCCACAGACGCCATTTCAACTTGAGTGGTCTGTATGTCTATTG
GCAAATCGAAAACCCAGAAGTTTCGGTGACTTACTACCTGCCTTA
CAAGTCTCCAGGGATCAATGTGGACAGTTCCTACAAGAACAGGGG
CCATCTGTCCCTGGACTCCATGAAGCAGGGTAACTTCTCTCTGTA
CCTGAAGAATGTCACCCCTCAGGATACCCAGGAGTTCACATGCCG
GGTATTTATGAATACAGCCACAGAGTTAGTCAAGATCTTGGAAGA
GGTGGTCAGGCTGCGTGTGGCAGCAAACTTCAGTACACCTGTCAT
CAGCACCTCTGATAGCTCCAACCCGGGCCAGGAACGTACCTACAC
CTGCATGTCCAAGAATGGCTACCCAGAGCCCAACCTGTATTGGAT
CAACACAACGGACAATAGCCTAATAGACACGGCTCTGCAGAATAA
CACTGTCTACTTGAACAAGTTGGGCCTGTATGATGTAATCAGCAC
ATTAAGGCTCCCTTGGACATCTCGTGGGGATGTTCTGTGCTGCGT
AGAGAATGTGGCTCTCCACCAGAACATCACTAGCATTAGCCAGGC
AGAAAGTTTCACTGGAAATAACACAAAGAACCCACAGGAAACCCA
CAATAATGAGTTAAAAGTCCTTGTCCCCGTCCTTGCTGTACTGGC
GGCAGCGGCATTCGTTTCCTTCATCATATACAGACGCACGCGTCC
CCACCGAAGCTATACAGGACCCAAGACTGTACAGCTTGAACTTAC
AGACACTTGGGCTCCCGTCCCCTACCAGGACTATTTGATTCCAAG
ATATTTGATGTCTCCATGCCTCAAAACACGTGGTTTACCATAA.
In another non-limiting example, the APC
may express human CD150 as set forth in
SEQ ID NO: 45:
MDPKGLLSLTFVLFLSLAFGASYGTGGRMMNCPKILRQLGSKVLL
PLTYERINKSMNKSIHIVVTMAKSLENSVENKIVSLDPSEAGPPR
YLGDRYKFYLENLTLGIRESRKEDEGWYLMTLEKNVSVQRFCLQL
RLYEQVSTPEIKVLNKTQENGTCTLILGCTVEKGDHVAYSWSEKA
GTHPLNPANSSHLLSLTLGPQHADNIYICTVSNPISNNSQTFSPW
PGCRTDPSETKPWAVYAGLLGGVIMILIMVVILQLRRRGKTNHYQ
TTVEKKSLTIYAQVQKPGPLQKKLDSFPAQDPCTTIYVAATEPVP
ESVQETNSITVYASVTLPES;
or as encoded by the DNA sequence set
forth in SEQ ID NO: 46:
ATGGATCCCAAGGGGCTCCTCTCCTTGACCTTCGTGCTGTTTCTC
TCCCTGGCTTTTGGGGCAAGCTACGGAACAGGTGGGCGCATGATG
AACTGCCCAAAGATTCTCCGGCAGTTGGGAAGCAAAGTGCTGCTG
CCCCTGACATATGAAAGGATAAATAAGAGCATGAACAAAAGCATC
CACATTGTCGTCACAATGGCAAAATCACTGGAGAACAGTGTCGAG
AACAAAATAGTGTCTCTTGATCCATCCGAAGCAGGCCCTCCACGT
TATCTAGGAGATCGCTACAAGTTTTATCTGGAGAATCTCACCCTG
GGGATACGGGAAAGCAGGAAGGAGGATGAGGGATGGTACCTTATG
ACCCTGGAGAAAAATGTTTCAGTTCAGCGCTTTTGCCTGCAGTTG
AGGCTTTATGAGCAGGTCTCCACTCCAGAAATTAAAGTTTTAAAC
AAGACCCAGGAGAACGGGACCTGCACCTTGATACTGGGCTGCACA
GTGGAGAAGGGGGACCATGTGGCTTACAGCTGGAGTGAAAAGGCG
GGCACCCACCCACTGAACCCAGCCAACAGCTCCCACCTCCTGTCC
CTCACCCTCGGCCCCCAGCATGCTGACAATATCTACATCTGCACC
GTGAGCAACCCTATCAGCAACAATTCCCAGACCTTCAGCCCGTGG
CCCGGATGCAGGACAGACCCCTCAGAAACAAAACCATGGGCAGTG
TATGCTGGGCTGTTAGGGGGTGTCATCATGATTCTCATCATGGTG
GTAATACTACAGTTGAGAAGAAGAGGTAAAACGAACCATTACCAG
ACAACAGTGGAAAAAAAAAGCCTTACGATCTATGCCCAAGTCCAG
AAACCAGGTCCTCTTCAGAAGAAACTTGACTCCTTCCCAGCTCAG
GACCCTTGCACCACCATATATGTTGCTGCCACAGAGCCTGTCCCA
GAGTCTGTCCAGGAAACAAATTCCATCACAGTCTATGCTAGTGTG
ACACTTCCAGAGAGCTGA.
In another non-limiting example, the APC
may express murine CD150 as set forth
in SEQ ID NO: 47:
MDPKGSLSWRILLFLSLAFELSYGTGGGVMDCPVILQKLGODTWL
PLTNEHQINKSVNKSVRILVTMATSPGSKSNKKIVSFDLSKGSYP
DHLEDGYHFQSKNLSLKILGNRRESEGWYLVSVEENVSVQQFCKQ
LKLYEQVSPPEIKVLNKTQENENGTCSLLLACTVKKGDHVTYSWS
DEAGTHLLSRANRSHLLHITLSNQHQDSIYNCTASNPVSSISRTF
NLSSQACKQESSSESSPWMQYTLVPLGVVIIFILVFTAIIMMKRQ
GKSNHCQPPVEEKSLTIYAQVQKSGPQEKKLHDALTDQDPCTTIY
VAATEPAPESVQEPNPTTVYASVTLPES;
or as encoded by the DNA sequence set
forth in SEQ ID NO: 48:
ATGGATCCCAAAGGATCCCTTTCCTGGAGAATACTTCTGTTTCTC
TCCCTGGCTTTTGAGTTGAGCTACGGAACAGGTGGAGGTGTGATG
GATTGCCCAGTGATTCTCCAGAAGCTGGGACAGGACACGTGGCTG
CCCCTGACGAATGAACATCAGATAAATAAGAGCGTGAACAAAAGT
GTCCGCATCCTCGTCACCATGGCGACGTCCCCAGGAAGCAAATCC
AACAAGAAAATTGTGTCTTTTGATCTCTCTAAAGGGAGCTATCCA
GATCACCTGGAGGATGGCTACCACTTTCAATCAAAAAACCTGAGC
CTGAAGATCCTCGGGAACAGGCGGGAGAGTGAAGGATGGTACTTG
GTGAGCGTGGAGGAGAACGTTTCTGTTCAGCAATTCTGCAAGCAG
CTGAAGCTTTATGAACAGGTCTCCCCTCCAGAGATTAAAGTGCTA
AACAAAACCCAGGAGAACGAGAATGGGACCTGCAGCTTGCTGTTG
GCCTGCACAGTGAAGAAAGGGGACCATGTGACTTACAGCTGGAGT
GATGAGGCAGGCACCCACCTGCTGAGCCGAGCCAACCGCTCCCAC
CTCCTGCACATCACTCTTAGCAACCAGCATCAAGACAGCATCTAC
AACTGCACCGCAAGCAACCCTGTCAGCAGTATCTCTAGGACCTTC
AACCTATCATCGCAAGCATGCAAGCAGGAATCCTCCTCAGAATCG
AGTCCATGGATGCAATATACTCTTGTACCACTGGGGGTCGTTATA
ATCTTCATCCTGGTTTTCACGGCAATAATAATGATGAAAAGACAA
GGTAAATCAAATCACTGCCAGCCACCAGTGGAAGAAAAAAGCCTT
ACTATTTATGCCCAAGTACAGAAATCAGGGCCTCAAGAGAAGAAA
CTTCATGATGCCCTAACAGATCAGGACCCCTGCACAACCATTTAT
GTGGCTGCCACAGAGCCTGCCCCAGAGTCTGTCCAGGAACCAAAC
CCCACCACAGTTTATGCCAGTGTGACACTGCCAGAGAGCTGA.
In another non-limiting example, the APC
may express human IL-12 as set forth
in SEQ ID NO: 49:
MWPPGSASQPPPSPAAATGLHPAARPVSLQCRLSMCPARSLLLVA
TLVLLDHLSLARNLPVATPDPGMFPCLHHSQNLLRAVSNMLQKNE
SCLNSRETSFITNGSCLASRKTSFMMALCLSSIYEDLKMYQVEFK
TMNAKLLMDPKRQIFLDQNMLAVIDELMQALNFNSETVPQKSSLE
EPDFYKTKIKLCILLHAFRIRAVTIDRVMSYLNAS;
or as encoded by the DNA sequence set
forth in SEQ ID NO: 50:
ATGTGGCCCCCTGGGTCAGCCTCCCAGCCACCGCCCTCACCTGCC
GCGGCCACAGGTCTGCATCCAGCGGCTCGCCCTGTGTCCCTGCAG
TGCCGGCTCAGCATGTGTCCAGCGCGCAGCCTCCTCCTTGTGGCT
ACCCTGGTCCTCCTGGACCACCTCAGTTTGGCCAGAAACCTCCCC
GTGGCCACTCCAGACCCAGGAATGTTCCCATGCCTTCACCACTCC
CAAAACCTGCTGAGGGCCGTCAGCAACATGCTCCAGAAGAATGAG
AGTTGCCTAAATTCCAGAGAGACCTCTTTCATAACTAATGGGAGT
TGCCTGGCCTCCAGAAAGACCTCTITTATGATGGCCCTGTGCCTT
AGTAGTATTTATGAAGACTTGAAGATGTACCAGGTGGAGTTCAAG
ACCATGAATGCAAAGCTTCTGATGGATCCTAAGAGGCAGATCTTT
CTAGATCAAAACATGCTGGCAGTTATTGATGAGCTGATGCAGGCC
CTGAATTTCAACAGTGAGACTGTGCCACAAAAATCCTCCCTTGAA
GAACCGGATTTTTATAAAACTAAAATCAAGCTCTGCATACTTCTT
CATGCTTTCAGAATTCGGGCAGTGACTATTGATAGAGTGATGAGC
TATCTGAATGCTTCCTAA.
In another non-limiting example, the APC
may express murine IL-12 as set forth
in SEQ ID NO: 51:
MCPQKLTISWFAIVLLVSPLMAMWELEKDVYVVEVDWTPDAPGET
VNLTCDTPEEDDITWTSDQRHGVIGSGKTLTITVKEFLDAGQYTC
HKGGETLSHSHLLLHKKENGIWSTEILKNFKNKTFLKCEAPNYSG
RFTCSWLVQRNMDLKFNIKSSSSSPDSRAVTCGMASLSAEKVTLD
QRDYEKYSVSCQEDVTCPTAEETLPIELALEARQQNKYENYSTSF
FIRDIIKPDPPKNLQMKPLKNSQVEVSWEYPDSWSTPHSYFSLKF
FVRIQRKKEKMKETEEGCNQKGAFLVEKTSTEVQCKGGNVCVQAQ
DRYYNSSCSKWACVPCRVRS;
or as encoded by the DNA sequence set
forth in SEQ ID NO: 52:
ATGTGTCCTCAGAAGCTAACCATCTCCTGGTTTGCCATCGTTTTG
CTGGTGTCTCCACTCATGGCCATGTGGGAGCTGGAGAAAGACGTT
TATGTTGTAGAGGTGGACTGGACTCCCGATGCCCCTGGAGAAACA
GTGAACCTCACCTGTGACACGCCTGAAGAAGATGACATCACCTGG
ACCTCAGACCAGAGACATGGAGTCATAGGCTCTGGAAAGACCCTG
ACCATCACTGTCAAAGAGTTTCTAGATGCTGGCCAGTACACCTGC
CACAAAGGAGGCGAGACTCTGAGCCACTCACATCTGCTGCTCCAC
AAGAAGGAAAATGGAATTTGGTCCACTGAAATTTTAAAAAATTTC
AAAAACAAGACTTTCCTGAAGTGTGAAGCACCAAATTACTCCGGA
CGGTTCACGTGCTCATGGCTGGTGCAAAGAAACATGGACTTGAAG
TTCAACATCAAGAGCAGTAGCAGTTCCCCTGACTCTCGGGCAGTG
ACATGTGGAATGGCGTCTCTGTCTGCAGAGAAGGTCACACTGGAC
CAAAGGGACTATGAGAAGTATTCAGTGTCCTGCCAGGAGGATGTC
ACCTGCCCAACTGCCGAGGAGACCCTGCCCATTGAACTGGCGTTG
GAAGCACGGCAGCAGAATAAATATGAGAACTACAGCACCAGCTTC
TTCATCAGGGACATCATCAAACCAGACCCGCCCAAGAACTTGCAG
ATGAAGCCTTTGAAGAACTCACAGGTGGAGGTCAGCTGGGAGTAC
CCTGACTCCTGGAGCACTCCCCATTCCTACTTCTCCCTCAAGTTC
TTTGTTCGAATCCAGCGCAAGAAAGAAAAGATGAAGGAGACAGAG
GAGGGGTGTAACCAGAAAGGTGCGTTCCTCGTAGAGAAGACATCT
ACCGAAGTCCAATGCAAAGGCGGGAATGTCTGCGTGCAAGCTCAG
GATCGCTATTACAATTCCTCATGCAGCAAGTGGGCATGTGTTCCC
TGCAGGGTCCGATCCTAG.
In another non-limiting example, the APC
may express human IL-7 as set forth in
SEQ ID NO: 53:
MFHVSFRYIFGLPPLILVLLPVASSDCDIEGKDGKQYESVLMVSI
DQLLDSMKEIGSNCLNNEFNFFKRHICDANKVKGRKPAALGEAQP
TKSLEENKSLKEQKKLNDLCFLKRLLQEIKTCWNKILMGTKEH;
or as encoded by the DNA sequence set forth in
SEQ ID NO: 54:
ATGTTCCATGTTTCTTTTAGGTATATCTTTGGACTTCCTCCCCTG
ATCCTTGTTCTGTTGCCAGTAGCATCATCTGATTGTGATATTGAA
GGTAAAGATGGCAAACAATATGAGAGTGTTCTAATGGTCAGCATC
GATCAATTATTGGACAGCATGAAAGAAATTGGTAGCAATTGCCTG
AATAATGAATTTAACTTTTTTAAAAGACATATCTGTGATGCTAAT
AAGGTTAAAGGAAGAAAACCAGCTGCCCTGGGTGAAGCCCAACCA
ACAAAGAGTTTGGAAGAAAATAAATCTTTAAAGGAACAGAAAAAA
CTGAATGACTTGTGTTTCCTAAAGAGACTATTACAAGAGATAAAA
ACTTGTTGGAATAAAATTTTGATGGGCACTAAAGAACACTG.
In another non-limiting example, the APC
may express murine IL-7 as set forth in
SEQ ID NO: 55:
MFHVSFRYIFGIPPLILVLLPVTSSECHIKDKEGKAYESVLMISI
DELDKMTGTDSNCPNNEPNFFRKHVCDDTKEAAFLNRAARKLKQF
LKMNISEEFNVHLLTVSQGTQTLVNCTSKEEKNVKEQKKNDACFL
KRLLREIKTCWNKILKGSI;
or as encoded by the DNA sequence set forth in
SEQ ID NO: 56:
ATGTTCCATGTTTCTTTTAGATATATCTTTGGAATTCCTCCACTG
ATCCTTGTTCTGCTGCCTGTCACATCATCTGAGTGCCACATTAAA
GACAAAGAAGGTAAAGCATATGAGAGTGTACTGATGATCAGCATC
GATGAATTGGACAAAATGACAGGAACTGATAGTAATTGCCCGAAT
AATGAACCAAACTTTTTTAGAAAACATGTATGTGATGATACAAAG
GAAGCTGCTTTTCTAAATCGTGCTGCTCGCAAGTTGAAGCAATTT
CTTAAAATGAATATCAGTGAAGAATTCAATGTCCACTTACTAACA
GTATCACAAGGCACACAAACACTGGTGAACTGCACAAGTAAGGAA
GAAAAAAACGTAAAGGAACAGAAAAAGAATGATGCATGTTTCCTA
AAGAGACTACTGAGAGAAATAAAAACTTGTTGGAATAAAATTTTG
AAGGGCAGTATATAA.
In another non-limiting example, the APC
may express human IL-15 as set forth in
SEQ ID NO: 57:
MRISKPHLRSISIQCYLCLLLNSHFLTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS;
or as encoded by the DNA sequence set forth in
SEQ ID NO: 58:
ATGAGAATTTCGAAACCACATTTGAGAAGTATTTCCATCCAGTGC
TACTTGTGTTTACTTCTAAACAGTCATTTTCTAACTGAAGCTGGC
ATTCATGTCTTCATTTTGGGCTGTTTCAGTGCAGGGCTTCCTAAA
ACAGAAGCCAACTGGGTGAATGTAATAAGTGATTTGAAAAAAATT
GAAGATCTTATTCAATCTATGCATATTGATGCTACTTTATATACG
GAAAGTGATGTTCACCCCAGTTGCAAAGTAACAGCAATGAAGTGC
TTTCTCTTGGAGTTACAAGTTATTTCACTTGAGTCCGGAGATGCA
AGTATTCATGATACAGTAGAAAATCTGATCATCCTAGCAAACAAC
AGTTTGTCTTCTAATGGGAATGTAACAGAATCTGGATGCAAAGAA
TGTGAGGAACTGGAGGAAAAAAATATTAAAGAATTTTTGCAGAGT
TTTGTACATATTGTCCAAATGTTCATCAACACTTCTTGA.
In another non-limiting example, the APC
may express human IL-17 as set forth in
SEQ ID NO: 59:
MDWPHNLLFLLTISIFLGLGQPRSPKSKRKGQGRPGPLAPGPHQV
PLDLVSRMKPYARMEEYERNIEEMVAQLRNSSELAQRKCEVNLQL
WMSNKRSLSPWGYSINHDPSRIPVDLPEARCLCLGCVNPFTMQED
RSMVSVPVFSQVPVRRRLCPPPPRTGPCRQRAVMETIAVGCTCIF;
or as encoded by the DNA sequence set forth in
SEQ ID NO: 60:
ATGGACTGGCCTCACAACCTGCTGTTTCTTCTTACCATTTCCATC
TTCCTGGGGCTGGGCCAGCCCAGGAGCCCCAAAAGCAAGAGGAAG
GGGCAAGGGCGGCCTGGGCCCCTGGCCCCTGGCCCTCACCAGGTG
CCACTGGACCTGGTGTCACGGATGAAACCGTATGCCCGCATGGAG
GAGTATGAGAGGAACATCGAGGAGATGGTGGCCCAGCTGAGGAAC
AGCTCAGAGCTGGCCCAGAGAAAGTGTGAGGTCAACTTGCAGCTG
TGGATGTCCAACAAGAGGAGCCTGTCTCCCTGGGGCTACAGCATC
AACCACGACCCCAGCCGTATCCCCGTGGACCTGCCGGAGGCACGG
TGCCTGTGTCTGGGCTGTGTGAACCCCTTCACCATGCAGGAGGAC
CGCAGCATGGTGAGCGTGCCGGTGTTCAGCCAGGTTCCTGTGCGC
CGCCGCCTCTGCCCGCCACCGCCCCGCACAGGGCCTTGCCGCCAG
CGCGCAGTCATGGAGACCATCGCTGTGGGCTGCACCTGCATCTTC
TGA.
In another non-limiting example, the APC
may express murine IL-17 as set forth in
SEQ ID NO: 61:
MSPGRASSVSLMLLLLLSLAATVKAAAIIPQSSACPNTEAKDFLQ
NVKVNLKVFNSLGAKVSSRRPSDYLNRSTSPWTLHRNEDPDRYPS
VIWEAQCRHQRCVNAEGKLDHHMNSVLIQQEILVLKREPESCPFT
FRVEKMLVGVGCTCVASIVRQAA;
or as encoded by the DNA sequence set forth in
SEQ ID NO: 62:
ATGAGTCCAGGGAGAGCTTCATCTGTGTCTCTGATGCTGTTGCTG
CTGCTGAGCCTGGCGGCTACAGTGAAGGCAGCAGCGATCATCCCT
CAAAGCTCAGCGTGTCCAAACACTGAGGCCAAGGACTTCCTCCAG
AATGTGAAGGTCAACCTCAAAGTCTTTAACTCCCTTGGCGCAAAA
GTGAGCTCCAGAAGGCCCTCAGACTACCTCAACCGTTCCACGTCA
CCCTGGACTCTCCACCGCAATGAAGACCCTGATAGATATCCCTCT
GTGATCTGGGAAGCTCAGTGCCGCCACCAGCGCTGTGTCAATGCG
GAGGGAAAGCTGGACCACCACATGAATTCTGTTCTCATCCAGCAA
GAGATCCTGGTCCTGAAGAGGGAGCCTGAGAGCTGCCCCTTCACT
TTCAGGGTCGAGAAGATGCTGGTGGGTGTGGGCTGCACCTGCGTG
GCCTCGATTGTCCGCCAGGCAGCCTAA.
In another non-limiting example, the APC
may express human IL-21 as set forth in
SEQ ID NO: 63:
MRSSPGNMERIVICLMVIFLGTLVHKSSSQGQDRHMIRMRQLIDI
VDQLKNYVNDLVPEFLPAPEDVETNCEWSAFSCFQKAQLKSANTG
NNERIINVSIKKLKRKPPSTNAGRRQKHRLTCPSCDSYEKKPPKE
FLERFKSLLQKMIHQHLSSRTHGSEDS;
or as encoded by the DNA sequence set forth in
SEQ ID NO: 64:
ATGAGATCCAGTCCTGGCAACATGGAGAGGATTGTCATCTGTCTG
ATGGTCATCTTCTTGGGGACACTGGTCCACAAATCAAGCTCCCAA
GGTCAAGATCGCCACATGATTAGAATGCGTCAACTTATAGATATT
GTTGATCAGCTGAAAAATTATGTGAATGACTTGGTCCCTGAATTT
CTGCCAGCTCCAGAAGATGTAGAGACAAACTGTGAGTGGTCAGCT
TTTTCCTGCTTTCAGAAGGCCCAACTAAAGTCAGCAAATACAGGA
AACAATGAAAGGATAATCAATGTATCAATTAAAAAGCTGAAGAGG
AAACCACCTTCCACAAATGCAGGGAGAAGACAGAAACACAGACTA
ACATGCCCTTCATGTGATTCTTATGAGAAAAAACCACCCAAAGAA
TTCCTAGAAAGATTCAAATCACTTCTCCAAAAGATGATTCATCAG
CATCTGTCCTCTAGAACACACGGAAGTGAAGATTCCTGA.
In another non-limiting example, the APC
may express murine IL-21 as set forth in
SEQ ID NO: 65:
MERTLVCLVVIFLGTVAHKSSPQGPDRLLIRLRHLIDIVEQLKIY
ENDLDPELLSAPQDVKGHCEHAAFACFQKAKLKPSNPGNNKTFII
DLVAQLRRRLPARRGGKKQKHIAKCPSCDSYEKRTPKEFLERLKW
LLQKMIHQHLS;
or as encoded by the DNA sequence set forth in
SEQ ID NO: 66:
ATGGAGAGGACCCTTGTCTGTCTGGTAGTCATCTTCTTGGGGACA
GTGGCCCATAAATCAAGCCCCCAAGGGCCAGATCGCCTCCTGATT
AGACTTCGTCACCTTATTGACATTGTTGAACAGCTGAAAATCTAT
GAAAATGACTTGGATCCTGAACTTCTATCAGCTCCACAAGATGTA
AAGGGGCACTGTGAGCATGCAGCTTTTGCCTGTTTTCAGAAGGCC
AAACTCAAGCCATCAAACCCTGGAAACAATAAGACATTCATCATT
GACCTCGTGGCCCAGCTCAGGAGGAGGCTGCCTGCCAGGAGGGGA
GGAAAGAAACAGAAGCACATAGCTAAATGCCCTTCCTGTGATTCG
TATGAGAAAAGGACACCCAAAGAATTCCTAGAAAGACTAAAATGG
CTCCTTCAAAAGATGATTCATCAGCATCTCTCCTAG.
In another non-limiting example, the APC
may express human IL-1 as set forth in
SEQ ID NO: 67:
MKVLLRLICFIALLISSLEADKCKEREEKIILVSSANEIDVRPCP
LNPNEHKGTITWYKDDSKTPVSTEQASRIHQHKEKLWFVPAKVED
SGHYYCVVRNSSYCLRIKISAKFVENEPNLCYNAQAIFKQKLPVA
GDGGLVCPYMEFFKNENNELPKLQWYKDCKPLLLDNIHFSGVKDR
LIVMNVAEKHRGNYTCHASYTYLGKQYPITRVIEFITLEENKPTR
PVIVSPANETMEVDLGSQIQLICNVTGQLSDIAYWKWNGSVIDED
DPVLGEDYYSVENPANKRRSTLITVLNISEIESRFYKHPFTCFAK
NTHGIDAAYIQLIYPVTNFQKHMIGICVTLTVIIVCSVFIYKIFK
IDIVLWYRDSCYDFLPIKASDGKTYDAYILYPKTVGEGSTSDCDI
FVFKVLPEVLEKQCGYKLFIYGRDDYVGEGMCVMEQSKGLLL;
or as encoded by the DNA sequence set forth in
SEQ ID NO: 68:
ATGAAAGTGTTACTCAGACTTATTTGTTTCATAGCTCTACTGATT
TCTTCTCTGGAGGCTGATAAATGCAAGGAACGTGAAGAAAAAATA
ATTTTAGTGTCATCTGCAAATGAAATTGATGTTCGTCCCTGTCCT
CTTAACCCAAATGAACACAAAGGCACTATAACTTGGTATAAAGAT
GACAGCAAGACACCTGTATCTACAGAACAAGCCTCCAGGATTCAT
CAACACAAAGAGAAACTTTGGTTTGTTCCTGCTAAGGTGGAGGAT
TCAGGACATTACTATTGCGTGGTAAGAAATTCATCTTACTGCCTC
AGAATTAAAATAAGTGCAAAATTTGTGGAGAATGAGCCTAACTTA
TGTTATAATGCACAAGCCATATTTAAGCAGAAACTACCCGTTGCA
GGAGACGGAGGACTTGTGTGCCCTTATATGGAGTTTTTTAAAAAT
GAAAATAATGAGTTACCTAAATTACAGTGGTATAAGGATTGCAAA
CCTCTACTTCTTGACAATATACACTTTAGTGGAGTCAAAGATAGG
CTCATCGTGATGAATGTGGCTGAAAAGCATAGAGGGAACTATACT
TGTCATGCATCCTACACATACTTGGGCAAGCAATATCCTATTACC
CGGGTAATAGAATTTATTACTCTAGAGGAAAACAAACCCACAAGG
CCTGTGATTGTGAGCCCAGCTAATGAGACAATGGAAGTAGACTTG
GGATCCCAGATACAATTGATCTGTAATGTCACCGGCCAGTTGAGT
GACATTGCTTACTGGAAGTGGAATGGGTCAGTAATTGATGAAGAT
GACCCAGTGCTAGGGGAAGACTATTACAGTGTGGAAAATCCTGCA
AACAAAAGAAGGAGTACCCTCATCACAGTGCTTAATATATCGGAA
ATTGAAAGTAGATTTTATAAACATCCATTTACCTGTTTTGCCAAG
AATACACATGGTATAGATGCAGCATATATCCAGTTAATATATCCA
GTCACTAATTTCCAGAAGCACATGATTGGTATATGTGTCACGTTG
ACAGTCATAATTGTGTGTTCTGTTTTCATCTATAAAATCTTCAAG
ATTGACATTGTGCTTTGGTACAGGGATTCCTGCTATGATTTTCTC
CCAATAAAAGCTTCAGATGGAAAGACCTATGACGCATATATACTG
TATCCAAAGACTGTTGGGGAAGGGTCTACCTCTGACTGTGATATT
TTTGTGTTTAAAGTCTTGCCTGAGGTCTTGGAAAAACAGTGTGGA
TATAAGCTGTTCATTTATGGAAGGGATGACTACGTTGGGGAAGGT
ATGTGTGTAATGGAACAGAGTAAAGGCTTATTGTTGTAA.
In another non-limiting example, the APC
may express murine IL-1 as set forth in
SEQ ID NO: 69:
MENMKVLLGLICLMVPLLSLEIDVCTEYPNQIVLFLSVNEIDIRK
CPLTPNKMHGDTIIWYKNDSKTPISADRDSRIHQQNEHLWFVPAK
VEDSGYYYCIVRNSTYCLKTKVTVTVLENDPGLCYSTQATFPQRL
HIAGDGSLVCPYVSYFKDENNELPEVQWYKNCKPLLLDNVSFFGV
KDKLLVRNVAEEHRGDYICRMSYTFRGKQYPVTRVIQFITIDENK
RDRPVILSPRNETIEADPGSMIQLICNVTGQFSDLVYWKWNGSEI
EWNDPFLAEDYQFVEHPSTKRKYTLITTLNISEVKSQFYRYPFIC
VVKNTNIFESAHVQLIYPVPDFKNYLIGGFIILTATIVCCVCIYK
VFKVDIVLWYRDSCSGFLPSKASDGKTYDAYILYPKTLGEGSFSD
LDTFVFKLLPEVLEGQFGYKLFIYGRDDYVGEDTIEVTNENVKKS
RRLIIILVRDMGGFSWLGQSSEEQIAIYNALIQEGIKIVLLELEK
IQDYEKMPDSIQFIKQKHGVICWSGDFQERPQSAKTRFWKNLRYQ
MPAQRRSPLSKHRLLTLDPVRDTKEKLPAATHLPLG;
or as encoded by the DNA sequence set forth in
SEQ ID NO: 70:
ATGGAGAATATGAAAGTGCTACTGGGGCTCATTTGTCTCATGGTG
CCTCTGCTGTCGCTGGAGATTGAGTATGTACAGAATATCCAAATC
AGATCGTTTTGTTTTTATCTGTAAATGAAATTGATATTCGCAAGT
GTCCTCTTACTCCAAATAAAATGCACGGCGACACCATAATTTGGT
ACAAGAATGACAGCAAGACCCCCATATCAGCGGACCGGGACTCCA
GGATTCATCAGCAGAATGAACATCTTTGGTTTGTACCTGCCAAGG
TGGAGGACTCAGGATATTACTATTGTATAGTAAGAAACTCAACTT
ACTGCCTCAAAACTAAAGTAACCGTAACTGTGTTAGAGAATGACC
CTGGCTTGTGTTACAGCACACAGGCCACCTTCCCACAGCGGCTCC
ACATTGCCGGGGATGGAAGTCTTGTGTGCCCTTATGTGAGTTATT
TTAAAGATGAAAATAATGAGTTACCCGAGGTCCAGTGGTATAAGA
ACTGTAAACCTCTGCTTCTTGACAACGTGAGCTTCTTCGGAGTAA
AAGATAAACTGTTGGTGAGGAATGTGGCTGAAGAGCACAGAGGGG
ACTATATATGCCGTATGTCCTATACGTTCCGGGGGAAGCAATATC
CGGTCACACGAGTAATACAATTTATCACAATAGATGAAAACAAGA
GGGACAGACCTGTTATCCTGAGCCCTCGGAATGAGACGATCGAAG
CTGACCCAGGTCAATGATACAACTGATCTGCAACGTCACGGGCCA
GTTCTCAGACCTTGTCTACTGGAAGTGGAATGGATCAGAAATTGA
ATGGAATGATCCATTTCTAGCTGAAGACTATCAATTTGTGGAACA
TCCTTCAACCAAAAGAAAATACACACTCATTACAACACTTAACAT
TTCAGAAGTTAAAAGCCAGTTTTATCGCTATCCGTTTATCTGTGT
TGTTAAGAACACAAATATTTTTGAGTCGGCGCATGTGCAGTTAAT
ATACCCAGTCCCTGACTTCAAGAATTACCTCATCGGGGGCTTTAT
CATCCTCACGGCTACAATTGTATGCTGTGTGTGCATCTATAAAGT
CTTCAAGGTTGACATAGTGCTTTGGTACAGGGACTCCTGCTCTGG
TTTTCTTCCTTCAAAAGCTTCAGATGGAAAGACATACGATGCCTA
TATTCTTTATCCCAAGACCCTGGGAGAGGGGTCCTTCTCAGACTT
AGATACTTTTGTTTTTAAACTGTTGCCTGAGGTCTTGGAGGGACA
GTTTGGATACAAGCTGTTCATTTATGGAAGGGATGACTATGTTGG
AGAAGATACCATCGAGGTTACTAATGAAAATGTAAAGAAAAGCAG
GAGGCTGATTATCATTCTAGTGAGAGATATGGGAGGCTTCAGCTG
GCTGGGCCAGTCATCTGAAGAGCAAATAGCCATATACAATGCTCT
CATCCAGGAAGGAATTAAAATCGTCCTGCTTGAGTTGGAGAAAAT
CCAAGACTATGAGAAAATGCCAGATTCTATTCAGTTCATTAAGCA
GAAACACGGAGTCATTTGCTGGTCAGGAGACTTTCAAGAAAGACC
ACAGTCTGCAAAGACCAGGTTCTGGAAAAACTTAAGATACCAGAT
GCCAGCCCAACGGAGATCACCATTGTCTAAACACCGCTTACTAAC
CCTGGATCCTGTGCGGGACACTAAGGAGAAACTGCCGGCAGCAAC
ACACTTACCACTCGGCTAG.
In another non-limiting example, the APC
may express human BCL-6 as set forth in
SEQ ID NO: 71:
MGSPAAPEGALGYVREFTRHSSDVLGNLNELRLRGILTDVTLLVG
GQPLRAHKAVLIACSGFFYSIFRGRAGVGVDVLSLPGGPEARGFA
PLLDFMYTSRLRLSPATAPAVLAAATYLQMEHVVQACHRFIQASY
EPLGISLRPLEAEPPTPPTAPPPGSPRRSEGHPDPPTESRSCSQG
PPSPASPDPKACNWKKYKYIVLNSQASQAGSLVGERSSGQPCPQA
RLPSGDEASSSSSSSSSSSEEGPIPGPQSRLSPTAATVQFKCGAP
ASTPYLLTSQAQDTSGSPSERARPLPGSEFFSCONCEAVAGCSSG
LDSLVPGDEDKPYKCQLCRSSFRYKGNLASHRTVHTGEKPYHCSI
CGARFNRPANLKTHSRIHSGEKPYKCETCGSRFVQVAHLRAHVLI
HTGEKPYPCPTCGTRFRHLQTLKSHVRIHTGEKPYHCDPCGLHFR
HKSQLRLHLRQKHGAATNTKVHYHILGGP;
or as encoded by the DNA sequence set forth in
SEQ ID NO: 72:
ATGGGTTCCCCCGCCGCCCCGGAGGGAGCGCTGGGCTACGTCCGC
GAGTTCACTCGCCACTCCTCCGACGTGCTGGGCAACCTCAACGAG
CTGCGCCTGCGCGGGATCCTCACTGACGTCACGCTGCTGGTTGGC
GGGCAACCCCTCAGAGCACACAAGGCAGTTCTCATCGCCTGCAGT
GGCTTCTTCTATTCAATTTTCCGGGGCCGTGCGGGAGTCGGGGTG
GACGTGCTCTCTCTGCCCGGGGGTCCCGAAGCGAGAGGCTTCGCC
CCTCTATTGGACTTCATGTACACTTCGCGCCTGCGCCTCTCTCCA
GCCACTGCACCAGCAGTCCTAGCGGCCGCCACCTATTTGCAGATG
GAGCACGTGGTCCAGGCATGCCACCGCTTCATCCAGGCCAGCTAT
GAACCTCTGGGCATCTCCCTGCGCCCCCTGGAAGCAGAACCCCCA
ACACCCCCAACGGCCCCTCCACCAGGTAGTCCCAGGCGCTCCGAA
GGACACCCAGACCCACCTACTGAATCTCGAAGCTGCAGTCAAGGC
CCCCCCAGTCCAGCCAGCCCTGACCCCAAGGCCTGCAACTGGAAA
AAGTACAAGTACATCGTGCTAAACTCTCAGGCCTCCCAAGCAGGG
AGCCTGGTCGGGGAGAGAAGTTCTGGTCAACCTTGCCCCCAAGCC
AGGCTCCCCAGTGGAGACGAGGCCTCCAGCAGCAGCAGCAGCAGC
AGCAGCAGCAGTGAAGAAGGACCCATTCCTGGTCCCCAGAGCAGG
CTCTCTCCAACTGCTGCCACTGTGCAGTTCAAATGTGGGGCTCCA
GCCAGTACCCCCTACCTCCTCACATCCCAGGCTCAAGACACCTCT
GGATCACCCTCTGAACGGGCTCGTCCACTACCGGGAAGTGAATTT
TTCAGCTGCCAGAACTGTGAGGCTGTGGCAGGGTGCTCATCGGGG
CTGGACTCCTTGGTTCCTGGGGACGAAGACAAACCCTATAAGTGT
CAGCTGTGCCGGTCTTCGTTCCGCTACAAGGGCAACCTTGCCAGT
CATCGTACAGTGCACACAGGGGAAAAGCCTTACCACTGCTCAATC
TGCGGAGCCCGTTTTAACCGGCCAGCAAACCTGAAAACGCACAGC
CGCATCCATTCGGGAGAGAAGCCGTATAAGTGTGAGACGTGCGGC
TCGCGCTTTGTACAGGTGGCACATCTGCGGGCGCACGTGCTGATC
CACACCGGGGAGAAGCCCTACCCTTGCCCTACCTGCGGAACCCGC
TTCCGCCACCTGCAGACCCTCAAGAGCCACGTTCGCATCCACACC
GGAGAGAAGCCTTACCACTGCGACCCCTGTGGCCTGCATTTCCGG
CACAAGAGTCAACTGCGGCTGCATCTGCGCCAGAAACACGGAGCT
GCTACCAACACCAAAGTGCACTACCACATTCTCGGGGGGCCCTAG.
In another non-limiting example, the APC
may express murine BCL-6 as set forth in
SEQ ID NO: 73:
MASPADSCIQFTRHASDVLLNLNRLRSRDILTDVVIVVSREQFRA
HKTVLMACSGLFYSIFTDQLKCNLSVINLDPEISPEGFCILLDFM
YTSRLNLREGNIMAVMTTAMYLQMEHVVDTCRKFIKASEAEMAPA
LKPPREEFLNSRMLMPHDIMAYRGREVVENNMPLRNTPGCESRAF
APPLYSGLSTPPASYPMYSHLPLSTFLFSDEELRDAPRMPVANPF
PKERALPCDSARQVPNEYSRPAMEVSPSLCHSNIYSPKEAVPEEA
RSDIHYSVPEGPKPAVPSARNAPYFPCDKASKEEERPSSEDEIAL
HFEPPNAPLNRKGLVSPQSPQKSDCQPNSPTESCSSKNACILQAS
GSPPAKSPTDPKACNWKKYKFIVLNSLNQNAKPEGSEQAELGRLS
PRAYPAPPACQPPMEPANLDLQSPTKLSASGEDSTIPQASRLNNL
VNRSLAGSPRSSSESHSPLYMHPPKCTSCGSQSPQHTEMCLHTAG
PTFPEEMGETQSEYSDSSCENGTFFCNECDCRFSEEASLKRHTLQ
THSDKPYKCDRCQASFRYKGNLASHKTVHTGEKPYRCNICGAQFN
RPANLKTHTRIHSGEKPYKCETCGARFVQVAHLRAHVLIHTGEKP
YPCEICGTRFRHLQTLKSHLRIHTGEKPYHCEKCNLHFRHKSQLR
LHLRQKHGAITNTKVQYRVSAADLPPELPKAC;
or as encoded by the DNA sequence set forth in
SEQ ID NO: 74:
ATGGCCTCCCCGGCTGACAGCTGTATCCAGTTTACCCGGCACGCT
AGTGATGTTCTTCTCAACCTTAATCGCCTCCGGAGTCGGGACATC
TTGACGGACGTTGTCATCGTGGTGAGCCGTGAGCAGTTTAGAGCC
CATAAGACAGTGCTCATGGCCTGCAGCGGCCTGTTCTACAGTATC
TTCACTGACCAGTTGAAATGCAACCTTAGTGTAATCAATCTAGAT
CCTGAAATCAGCCCTGAGGGGTTTTGCATCCTCCTGGACTTCATG
TACACATCTAGGCTCAACCTGAGGGAAGGCAATATCATGGCGGTG
ATGACCACAGCCATGTACCTGCAGATGGAGCATGTTGTCGACACA
TGCAGGAAGTTCATCAAGGCCAGTGAAGCAGAAATGGCCCCTGCA
CTTAAACCTCCCCGTGAAGAGTTCCTGAACAGCCGGATGCTGATG
CCCCATGACATCATGGCCTACCGAGGTCGTGAGGTCGTGGAGAAC
AATATGCCACTGAGAAATACTCCCGGGTGTGAGAGCAGAGCTTTT
GCTCCTCCTCTGTACAGTGGCCTGTCAACACCACCAGCCTCTTAT
CCCATGTACAGCCATCTCCCGCTCAGCACCTTCCTCTTCTCTGAT
GAGGAGCTCCGAGATGCCCCCCGAATGCCTGTGGCCAACCCTTTT
CCCAAGGAGCGTGCCCTCCCCTGCGACAGTGCCAGGCAAGTCCCT
AATGAGTATAGCAGGCCAGCCATGGAGGTGTCCCCCAGTTTGTGT
CACAGCAACATCTACTCGCCCAAGGAGGCAGTCCCAGAGGAGGCT
CGGAGTGACATACACTACAGTGTGCCTGAGGGCCCCAAGCCTGCT
GTCCCTTCTGCTCGGAATGCTCCATACTTCCCCTGTGACAAAGCC
AGCAAAGAAGAAGAGAGACCTTCTTCGGAGGATGAGATTGCCCTG
CATTTCGAGCCCCCCAATGCACCCTTGAACCGGAAGGGTCTGGTT
AGTCCCCAGAGTCCCCAGAAATCCGACTGCCAGCCCAACTCACCC
ACAGAGTCCTGCAGCAGCAAGAACGCCTGCATCCTTCAGGCCTCT
GGCTCTCCGCCAGCCAAGAGCCCCACTGACCCGAAAGCCTGCAAC
TGGAAGAAGTATAAGTTCATCGTTCTCAACAGCCTCAATCAGAAT
GCCAAACCCGAGGGCTCTGAGCAGGCAGAGCTGGGTCGCCTCTCC
CCTCGAGCCTACCCTGCACCGCCCGCTTGCCAGCCGCCTATGGAG
CCCGCGAACCTTGATCTCCAGTCCCCGACCAAGCTCAGTGCCAGT
GGGGAGGACTCTACCATCCCCCAAGCCAGCCGGCTCAATAATCTC
GTGAACAGGTCCCTGGCAGGCTCCCCCCGAAGCAGCAGTGAGAGT
CACTCACCACTCTACATGCACCCCCCAAAGTGCACATCCTGCGGC
TCTCAGTCCCCACAGCATACAGAGATGTGCCTCCATACTGCTGGG
CCCACGTTCCCGGAGGAGATGGGGGAAACCCAGTCAGAGTATTCG
GATTCTAGCTGTGAGAATGGGACCTTCTTCTGCAACGAATGTGAC
TGCCGTTTCTCTGAGGAGGCCTCGCTCAAGAGGCACACGCTGCAG
ACGCACAGTGACAAACCATACAAATGTGATCGCTGCCAGGCCTCC
TTCCGCTACAAGGGCAACCTCGCCAGCCACAAGACTGTCCACACG
GGTGAGAAACCCTATCGCTGTAACATTTGTGGAGCGCAGTTCAAT
CGGCCAGCCAACCTGAAGACCCACACTCGAATTCACTCTGGAGAA
AAGCCCTACAAATGTGAAACCTGTGGGGCCAGGTTTGTTCAGGTG
GCCCACCTCCGTGCCCACGTGCTCATCCACACTGGAGAGAAGCCG
TACCCCTGTGAAATCTGTGGCACTCGCTTCCGGCACCTTCAGACT
CTGAAGAGCCATCTGCGCATCCACACAGGAGAGAAACCTTACCAT
TGTGAGAAGTGTAACCTGCACTTTCGTCACAAAAGCCAACTGCGA
CTTCATTTGCGCCAGAAGCACGGCGCCATCACCAACACCAAGGTG
CAATACCGCGTGTCGGCCGCTGACCTGCCTCCGGAGCTCCCCAAA
GCCTGCTGA.
In another non-limiting example, the APC
may express human BCLXL as set forth in
SEQ ID NO: 75:
MSQSNRELVVDFLSYKLSQKGYSWSQFSDVEENRTEAPEGTESEM
ETPSAINGNPSWHLADSPAVNGATGHSSSLDAREVIPMAAVKQAL
REAGDEFELRYRRAFSDLTSQLHITPGTAYQSFEQVVNELFRDGV
NWGRIVAFFSFGGALCVESVDKEMQVLVSRIAAWMATYLNDHLEP
WIQENGGWDTFVELYGNNAAAESRKGQERFNRWFLTGMTVAGVVL
LGSLFSRK;
or as encoded by the DNA sequence set forth in
SEQ ID NO: 76:
ATGTCTCAGAGCAACCGGGAGCTGGTGGTTGACTTTCTCTCCTAC
AAGCTTTCCCAGAAAGGATACAGCTGGAGTCAGTTTAGTGATGTG
GAAGAGAACAGGACTGAGGCCCCAGAAGGGACTGAATCGGAGATG
GAGACCCCCAGTGCCATCAATGGCAACCCATCCTGGCACCTGGCA
GACAGCCCCGCGGTGAATGGAGCCACTGGCCACAGCAGCAGTTTG
GATGCCCGGGAGGTGATCCCCATGGCAGCAGTAAAGCAAGCGCTG
AGGGAGGCAGGCGACGAGTTTGAACTGCGGTACCGGCGGGCATTC
AGTGACCTGACATCCCAGCTCCACATCACCCCAGGGACAGCATAT
CAGAGCTTTGAACAGGTAGTGAATGAACTCTTCCGGGATGGGGTA
AACTGGGGTCGCATTGTGGCCTTTTTCTCCTTCGGCGGGGCACTG
TGCGTGGAAAGCGTAGACAAGGAGATGCAGGTATTGGTGAGTCGG
ATCGCAGCTTGGATGGCCACTTACCTGAATGACCACCTAGAGCCT
TGGATCCAGGAGAACGGCGGCTGGGATACTTTTGTGGAACTCTAT
GGGAACAATGCAGCAGCCGAGAGCCGAAAGGGCCAGGAACGCTTC
AACCGCTGGTTCCTGACGGGCATGACTGTGGCCGGCGTGGTTCTG
CTGGGCTCACTCTTCAGTCGGAAATGA.
In another non-limiting example, the APC
may express murine BCLXL as set forth in
SEQ ID NO: 77:
MSQSNRELVVDFLSYKLSQKGYSWSQFSDVEENRTEAPEETEAER
ETPSAINGNPSWHLADSPAVNGATGHSSSLDAREVIPMAAVKQAL
REAGDEFELRYRRAFSDLTSQLHITPGTAYQSFEQVVNELFRDGV
NWGRIVAFFSFGGALCVESVDKEMQVLVSRIASWMATYLNDHLEP
WIQENGGWDTFVDLYGNNAAAESRKGQERFNRWFLTGMTVAGVVL
LGSLFSRK;
or as encoded by the DNA sequence set forth in
SEQ ID NO: 78:
ATGTCTCAGAGCAACCGGGAGCTGGTGGTCGACTTTCTCTCCTAC
AAGCTTTCCCAGAAAGGATACAGCTGGAGTCAGTTTAGTGATGTC
GAAGAGAATAGGACTGAGGCCCCAGAAGAAACTGAAGCAGAGAGG
GAGACCCCCAGTGCCATCAATGGCAACCCATCCTGGCACCTGGCG
GATAGCCCGGCCGTGAATGGAGCCACTGGCCACAGCAGCAGTTTG
GATGCGCGGGAGGTGATTCCCATGGCAGCAGTGAAGCAAGCGCTG
AGAGAGGCAGGCGATGAGTTTGAACTGCGGTACCGGAGAGCGTTC
AGTGATCTAACATCCCAGCTTCACATAACCCCAGGGACCGCGTAT
CAGAGCTTTGAGCAGGTAGTGAATGAACTCTTTCGGGATGGAGTA
AACTGGGGTCGCATCGTGGCCTTTTTCTCCTTTGGCGGGGCACTG
TGCGTGGAAAGCGTAGACAAGGAGATGCAGGTATTGGTGAGTCGG
ATTGCAAGTTGGATGGCCACCTATCTGAATGACCACCTAGAGCCT
TGGATCCAGGAGAACGGCGGCTGGGACACTTTTGTGGATCTCTAC
GGGAACAATGCAGCAGCCGAGAGCCGGAAAGGCCAGGAGCGCTTC
AACCGCTGGTTCCTGACGGGCATGACTGTGGCTGGTGTGGTTCTG
CTGGGCTCACTCTTCAGTCGGAAGTGA.
In another non-limiting example, the APC
may express human BCL 2 as set forth in
SEQ ID NO: 79:
MAHAGRTGYDNREIVMKYIHYKLSQRGYEWDAGDVGAAPPGAAPA
PGIFSSQPGHTPHPAASRDPVARTSPLQTPAAPGAAAGPALSPVP
PVVHLTLRQAGDDFSRRYRRDFAEMSSQLHLTPFTARGRFATVVE
ELFRDGVNWGRIVAFFEFGGVMCVESVNREMSPLVDNIALWMTEY
LNRHLHTWIQDNGGWDAFVELYGPSMRPLFDFSWLSLKTLLSLA;
or as encoded by the DNA sequence set forth in
SEQ ID NO: 80:
ATGGCGCACGCTGGGAGAACAGGGTACGATAACCGGGAGATAGTG
ATGAAGTACATCCATTATAAGCTGTCGCAGAGGGGCTACGAGTGG
GATGCGGGAGATGTGGGCGCCGCGCCCCCGGGGGCCGCCCCCGCA
CCGGGCATCTTCTCCTCCCAGCCCGGGCACACGCCCCATCCAGCC
GCATCCCGGGACCCGGTCGCCAGGACCTCGCCGCTGCAGACCCCG
GCTGCCCCCGGCGCCGCCGCGGGGCCTGCGCTCAGCCCGGTGCCA
CCTGTGGTCCACCTGACCCTCCGCCAGGCCGGCGACGACTTCTCC
CGCCGCTACCGCCGCGACTTCGCCGAGATGTCCAGCCAGCTGCAC
CTGACGCCCTTCACCGCGCGGGGACGCTTTGCCACGGTGGTGGAG
GAGCTCTTCAGGGACGGGGTGAACTGGGGGAGGATTGTGGCCTTC
TTTGAGTTCGGTGGGGTCATGTGTGTGGAGAGCGTCAACCGGGAG
ATGTCGCCCCTGGTGGACAACATCGCCCTGTGGATGACTGAGTAC
CTGAACCGGCACCTGCACACCTGGATCCAGGATAACGGAGGCTGG
GATGCCTTTGTGGAACTGTACGGCCCCAGCATGCGGCCTCTGTTT
GATTTCTCCTGGCTGTCTCTGAAGACTCTGCTCAGTTTGGCCCTG
GTGGGAGCTTGCATCACCCTGGGTGCCTATCTGGGCCACAAGTGA.
In another non-limiting example, the APC
may express murine BCL 2 as set forth in
SEQ ID NO: 81:
MAQAGRTGYDNREIVMKYIHYKLSQRGYEWDAGDADAAPLGAAPT
PGIFSFQPESNPMPAVHRDMAARTSPLRPLVATAGPALSPVPPVV
HLTLRRAGDDFSRRYRRDFAEMSSQLHLTPFTARGRFATVVEELF
RDGVNWGRIVAFFEFGGVMCVESVNREMSPLVDNIALWMTEYLNR
HLHTWIQDNGGWDAFVELYGPSMRPLFDFSWLSLKTLL;
or as encoded by the DNA sequence set forth in
SEQ ID NO: 82:
ATGGCGCAAGCCGGGAGAACAGGGTATGATAACCGGGAGATCGTG
ATGAAGTACATACATTATAAGCTGTCACAGAGGGGCTACGAGTGG
GATGCTGGAGATGCGGACGCGGCGCCCCTGGGGGCTGCCCCCACC
CCTGGCATCTTCTCCTTCCAGCCTGAGAGCAACCCAATGCCCGCT
GTGCACCGGGACATGGCTGCCAGGACGTCTCCTCTCAGGCCCCTC
GTTGCCACCGCTGGGCCTGCGCTCAGCCCTGTGCCACCTGTGGTC
CATCTGACCCTCCGCCGGGCTGGGGATGACTTCTCTCGTCGCTAC
CGTCGTGACTTCGCAGAGATGTCCAGTCAGCTGCACCTGACGCCC
TTCACCGCGAGGGGACGCTTTGCCACGGTGGTGGAGGAACTCTTC
AGGGATGGGGTGAACTGGGGGAGGATTGTGGCCTTCTTTGAGTTC
GGTGGGGTCATGTGTGTGGAGAGCGTCAACAGGGAGATGTCACCC
CTGGTGGACAACATCGCCCTGTGGATGACTGAGTACCTGAACCGG
CATCTGCACACCTGGATCCAGGATAACGGAGGCTGGGATGCCTTT
GTGGAACTATATGGCCCCAGCATGCGACCTCTGTTTGATTTCTCC
TGGCTGTCTCTGAAGACCCTGCTCAGCCTGGCCCTGGTCGGGGCC
TGCATCACTCTGGGTGCATACCTGGGCCACAAGTGA.
In another non-limiting example, the APC
may express human MCL 1 as set forth in
SEQ ID NO: 83:
MFGLKRNAVIGLNLYCGGAGLGAGSGGATRPGGRLLATEKEASAR
REIGGGEAGAVIGGSAGASPPSTLTPDSRRVARPPPIGAEVPDVT
ATPARLLFFAPTRRAAPLEEMEAPAADAIMSPEEELDGYEPEPLG
KRPAVLPLLELVGESGNNTSTDGSLPSTPPPAEEEEDELYRQSLE
IISRYLREQATGAKDTKPMGRSGATSRKALETLRRVGDGVQRNHE
TAFQGMLRKLDIKNEDDVKSLSRVMIHVFSDGVTNWGRIVTLISF
GAFVAKHLKTINQESCIEPLAESITDVLVRTKRDWLVKQRGWDGF
VEFFHVEDLEGGIRNVLLAFAGVAGVGAGLAYLIR;
or as encoded by the DNA sequence set forth in
SEQ ID NO: 84:
ATGTTTGGCCTCAAAAGAAACGCGGTAATCGGACTCAACCTCTAC
TGTGGGGGGGCCGGCTTGGGGGCCGGCAGCGGCGGCGCCACCCGC
CCGGGAGGGCGACTTTTGGCTACGGAGAAGGAGGCCTCGGCCCGG
CGAGAGATAGGGGGAGGGGAGGCCGGCGCGGTGATTGGCGGAAGC
GCCGGCGCAAGCCCCCCGTCCACCCTCACGCCAGACTCCCGGAGG
GTCGCGCGGCCGCCGCCCATTGGCGCCGAGGTCCCCGACGTCACC
GCGACCCCCGCGAGGCTGCTTTTCTTCGCGCCCACCCGCCGCGCG
GCGCCGCTTGAGGAGATGGAAGCCCCGGCCGCTGACGCCATCATG
TCGCCCGAAGAGGAGCTGGACGGGTACGAGCCGGAGCCTCTCGGG
AAGCGGCCGGCTGTCCTGCCGCTGCTGGAGTTGGTCGGGGAATCT
GGTAATAACACCAGTACGGACGGGTCACTACCCTCGACGCCGCCG
CCAGCAGAGGAGGAGGAGGACGAGTTGTACCGGCAGTCGCTGGAG
ATTATCTCTCGGTACCTTCGGGAGCAGGCCACCGGCGCCAAGGAC
ACAAAGCCAATGGGCAGGTCTGGGGCCACCAGCAGGAAGGCGCTG
GAGACCTTACGACGGGTTGGGGATGGCGTGCAGCGCAACCACGAG
ACGGCCTTCCAAGGCATGCTTCGGAAACTGGACATCAAAAACGAA
GACGATGTGAAATCGTTGTCTCGAGTGATGATCCATGTTTTCAGC
GACGGCGTAACAAACTGGGGCAGGATTGTGACTCTCATTTCTTTT
GGTGCCTTTGTGGCTAAACACTTGAAGACCATAAACCAAGAAAGC
TGCATCGAACCATTAGCAGAAAGTATCACAGACGTTCTCGTAAGG
ACAAAACGGGACTGGCTAGTTAAACAAAGAGGCTGGGATGGGTTT
GTGGAGTTCTTCCATGTAGAGGACCTAGAAGGTGGCATCAGGAAT
GTGCTGCTGGCTTTTGCAGGTGTTGCTGGAGTAGGAGCTGGTTTG
GCATATCTAATAAGATAG.
In another non-limiting example, the APC
may express murine MCL 1 as set forth in
SEQ ID NO: 85:
MFGLRRNAVIGLNLYCGGASLGAGGGSPAGARLVAEEAKARREGG
GEAALLPGARVVARPPPVGAEDPDVTASAERRLHKSPGLLAVPPE
EMAASAAAAIVSPEEELDGCEPEAIGKRPAVLPLLERVSEAAKSS
GADGSLPSTPPPPEEEEDDLYRQSLEIISRYLREQATGSKDSKPL
GEAGAAGRRALETLRRVGDGVQRNHETAFQGMLRKLDIKNEGDVK
SFSRVMVHVFKDGVTNWGRIVTLISFGAFVAKHLKSVNQESFIEP
LAETITDVLVRTKRDWLVKQRGWDGFVEFFHVQDLEGGIRNVLLA
FAGVAGVGAGLAYLIR;
or as encoded by the DNA sequence set forth in
SEQ ID NO: 86:
ATGTTTGGCCTGCGGAGAAACGCGGTCATCGGCTTGAACCTGTAC
TGCGGCGGCGCCAGCCTCGGCGCGGGCGGCGGTTCTCCGGCAGGG
GCGCGCCTGGTGGCCGAGGAGGCCAAGGCGCGGCGCGAGGGGGGA
GGGGAGGCCGCCCTGCTGCCCGGCGCGCGGGTGGTCGCCCGGCCG
CCGCCCGTGGGCGCCGAGGACCCCGACGTCACCGCGTCGGCCGAA
AGGCGGCTGCATAAGTCGCCCGGCCTCCTCGCCGTGCCGCCCGAG
GAGATGGCCGCGTCGGCCGCCGCCGCCATCGTGTCTCCGGAGGAG
GAACTGGACGGCTGCGAGCCGGAGGCCATCGGCAAGCGCCCGGCC
GTGCTGCCCCTCCTGGAGCGCGTGAGCGAGGCGGCCAAGAGCTCC
GGGGCCGACGGCTCTCTGCCCTCCACGCCGCCGCCGCCCGAGGAG
GAAGAGGACGACCTATACCGCCAGTCGCTGGAGATCATCTCGCGC
TACTTGCGGGAGCAGGCGACCGGCTCCAAGGACTCGAAGCCTCTG
GGCGAGGCGGGCGCGGCGGGCCGGAGAGCGCTGGAGACCCTGCGG
CGCGTGGGCGACGGCGTGCAGCGCAACCACGAGACGGCCTTCCAG
GGCATGCTCCGGAAACTGGACATTAAAAACGAAGGCGATGTTAAA
TCTTTTTCTCGAGTAATGGTCCATGTTTTCAAAGATGGCGTAACA
AACTGGGGCAGGATTGTGACTCTTATTTCTTTCGGTGCCTTTGTG
GCCAAACACTTAAAGAGCGTAAACCAAGAAAGCTTCATCGAACCA
TTAGCAGAAACTATCACAGATGTTCTTGTAAGGACGAAACGGGAC
TGGCTTGTCAAACAAAGAGGCTGGGATGGGTTTGTGGAGTTCTTC
CACGTACAGGACCTAGAAGGCGGCATCAGAAATGTGCTGCTGGCT
TTTGCGGGTGTTGCTGGAGTAGGGGCTGGTCTGGCATATCTAATA
AGATAG.
In another non-limiting example, the APC
may express human IL-2 as set forth in
SEQ ID NO: 87:
MYRMQLLSCIALSLALVTNSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT;
or as encoded by the DNA sequence set forth in
SEQ ID NO: 88:
ATGTACAGGATGCAACTCCTGTCTTGCATTGCACTAAGTCTTGCA
CTTGTCACAAACAGTGCACCTACTTCAAGTTCTACAAAGAAAACA
CAGCTACAACTGGAGCATTTACTGCTGGATTTACAGATGATTTTG
AATGGAATTAATAATTACAAGAATCCCAAACTCACCAGGATGCTC
ACATTTAAGTTTTACATGCCCAAGAAGGCCACAGAACTGAAACAT
CTTCAGTGTCTAGAAGAAGAACTCAAACCTCTGGAGGAAGTGCTA
AATTTAGCTCAAAGCAAAAACTTTCACTTAAGACCCAGGGACTTA
ATCAGCAATATCAACGTAATAGTTCTGGAACTAAAGGGATCTGAA
ACAACATTCATGTGTGAATATGCTGATGAGACAGCAACCATTGTA
GAATTTCTGAACAGATGGATTACCTTTTGTCAAAGCATCATCTCA
ACACTGACTTGA.
In another non-limiting example, the APC
may express murine IL-2 as set forth in
SEQ ID NO: 89:
MYSMQLASCVTLTLVLLVNSAPTSSSTSSSTAEAQQQQQQQQQQQ
QHLEQLLMDLQELLSRMENYRNLKLPRMLTFKFYLPKQATELKDL
QCLEDELGPLRHVLDLTQSKSFQLEDAENFISNIRVTVVKLKGSD
NTFECQFDDES;
or as encoded by the DNA sequence set forth in
SEQ ID NO: 90:
ATGTACAGCATGCAGCTCGCATCCTGTGTCACATTGACACTTGTG
CTCCTTGTCAACAGCGCACCCACTTCAAGCTCCACTTCAAGCTCT
ACAGCGGAAGCACAGCAGCAGCAGCAGCAGCAGCAGCAGCAGCAG
CAGCACCTGGAGCAGCTGTTGATGGACCTACAGGAGCTCCTGAGC
AGGATGGAGAATTACAGGAACCTGAAACTCCCCAGGATGCTCACC
TTCAAATTTTACTTGCCCAAGCAGGCCACAGAATTGAAAGATCTT
CAGTGCCTAGAAGATGAACTTGGACCTCTGCGGCATGTTCTGGAT
TTGACTCAAAGCAAAAGCTTTCAATTGGAAGATGCTGAGAATTTC
ATCAGCAATATCAGAGTAACTGTTGTAAAACTAAAGGGCTCTGAC
AACACATTTGAGTGCCAATTCGATGATGAGTCAGCAACTGTGGTG
GACTTTCTGAGGAGATGGATAGCCTTCTGTCAAAGCATCATCTCA
ACAAGCCCTCAATAA.
In another non-limiting example, the APC
may express human CD40L as set forth in
SEQ ID NO: 91:
MIETYNQTSPRSAATGLPISMKIFMYLLTVFLITQMIGSALFAVY
LHRRLDKIEDERNLHEDFVFMKTIQRCNTGERSLSLLNCEEIKSQ
FEGFVKDIMLNKEETKKENSFEMQKGDQNPQIAAHVISEASSKTT
SVLQWAEKGYYTMSNNLVTLENGKQLTVKRQGLYYIYAQVTFCSN
REASSQAPFIASLCLKSPGRFERILLRAANTHSSAKPCGQQSIHL
GGVFELQPGASVFVNVTDPSQVSHGTGFTSFGLLKL;
or as encoded by the DNA sequence set forth in
SEQ ID NO: 92:
ATGATCGAAACATACAACCAAACTTCTCCCCGATCTGCGGCCACT
GGACTGCCCATCAGCATGAAAATTTTTATGTATTTACTTACTGTT
TTTCTTATCACCCAGATGATTGGGTCAGCACTTTTTGCTGTGTAT
CTTCATAGAAGGTTGGACAAGATAGAAGATGAAAGGAATCTTCAT
GAAGATTTTGTATTCATGAAAACGATACAGAGATGCAACACAGGA
GAAAGATCCTTATCCTTACTGAACTGTGAGGAGATTAAAAGCCAG
TTTGAAGGCTTTGTGAAGGATATAATGTTAAACAAAGAGGAGACG
AAGAAAGAAAACAGCTTTGAAATGCAAAAAGGTGATCAGAATCCT
CAAATTGCGGCACATGTCATAAGTGAGGCCAGCAGTAAAACAACA
TCTGTGTTACAGTGGGCTGAAAAAGGATACTACACCATGAGCAAC
AACTTGGTAACCCTGGAAAATGGGAAACAGCTGACCGTTAAAAGA
CAAGGACTCTATTATATCTATGCCCAAGTCACCTTCTGTTCCAAT
CGGGAAGCTTCGAGTCAAGCTCCATTTATAGCCAGCCTCTGCCTA
AAGTCCCCCGGTAGATTCGAGAGAATCTTACTCAGAGCTGCAAAT
ACCCACAGTTCCGCCAAACCTTGCGGGCAACAATCCATTCACTTG
GGAGGAGTATTTGAATTGCAACCAGGTGCTTCGGTGTTTGTCAAT
GTGACTGATCCAAGCCAAGTGAGCCATGGCACTGGCTTCACGTCC
TTTGGCTTACTCAAACTCTGA.
In another non-limiting example, the APC
may express murine CD40L as set forth in
SEQ ID NO: 93:
MIETYSQPSPRSVATGLPASMKIFMYLLTVFLITQMIGSVLFAVY
LHRRLDKVEEEVNLHEDFVFIKKLKRCNKGEGSLSLLNCEEMRRQ
FEDLVKDITLNKEEKKENSFEMQRGDEDPQIAAHVVSEANSNAAS
VLQWAKKGYYTMKSNLVMLENGKQLTVKREGLYYVYTQVTFCSNR
EPSSQRPFIVGLWLKPSSGSERILLKAANTHSSSQLCEQQSVHLG
GVFELQAGASVFVNVTEASQVIHRVGFSSFGLLKL;
or as encoded by the DNA sequence set forth in
SEQ ID NO: 94:
ATGATAGAAACATACAGCCAACCTTCCCCCAGATCCGTGGCAACT
GGACTTCCAGCGAGCATGAAGATTTTTATGTATTTACTTACTGTT
TTCCTTATCACCCAAATGATTGGATCTGTGCTTTTTGCTGTGTAT
CTTCATAGAAGATTGGATAAGGTCGAAGAGGAAGTAAACCTTCAT
GAAGATTTTGTATTCATAAAAAAGCTAAAGAGATGCAACAAAGGA
GAAGGATCTTTATCCTTGCTGAACTGTGAGGAGATGAGAAGGCAA
TTTGAAGACCTTGTCAAGGATATAACGTTAAACAAAGAAGAGAAA
AAAGAAAACAGCTTTGAAATGCAAAGAGGTGATGAGGATCCTCAA
ATTGCAGCACACGTTGTAAGCGAAGCCAACAGTAATGCAGCATCC
GTTCTACAGTGGGCCAAGAAAGGATATTATACCATGAAAAGCAAC
TTGGTAATGCTTGAAAATGGGAAACAGCTGACGGTTAAAAGAGAA
GGACTCTATTATGTCTACACTCAAGTCACCTTCTGCTCTAATCGG
GAGCCTTCGAGTCAACGCCCATTCATCGTCGGCCTCTGGCTGAAG
CCCAGCAGTGGATCTGAGAGAATCTTACTCAAGGCGGCAAATACC
CACAGTTCCTCCCAGCTTTGCGAGCAGCAGTCTGTTCACTTGGGC
GGAGTGTTTGAATTACAAGCTGGTGCTTCTGTGTTTGTCAACGTG
ACTGAAGCAAGCCAAGTGATCCACAGAGTTGGCTTCTCATCTTTT
GGCTTACTCAAACTCTGA.
In another non-limiting example, the APC
may express human GITR-L as set forth in
SEQ ID NO: 95:
MCLSHLENMPLSHSRTQGAQRSSWKLWLFCSIVMLLFLCSFSWLI
FIFLQLETAKEPCMAKFGPLPSKWQMASSEPPCVNKVSDWKLEIL
QNGLYLIYGQVAPNANYNDVAPFEVRLYKNKDMIQTLTNKSKIQN
VGGTYELHVGDTIDLI;
or as encoded by the DNA sequence set forth in
SEQ ID NO: 96:
ATGTGTTTGAGCCACTTGGAAAATATGCCTTTAAGCCATTCAAGA
ACTCAAGGAGCTCAGAGATCATCCTGGAAGCTGTGGCTCTTTTGC
TCAATAGTTATGTTGCTATTTCTTTGCTCCTTCAGTTGGCTAATC
TTTATTTTTCTCCAATTAGAGACTGCTAAGGAGCCCTGTATGGCT
AAGTTTGGACCATTACCCTCAAAATGGCAAATGGCATCTTCTGAA
CCTCCTTGCGTGAATAAGGTGTCTGACTGGAAGCTGGAGATACTT
CAGAATGGCTTATATTTAATTTATGGCCAAGTGGCTCCCAATGCA
AACTACAATGATGTAGCTCCTTTTGAGGTGCGGCTGTATAAAAAC
AAAGACATGATACAAACTCTAACAAACAAATCTAAAATCCAAAAT
GTAGGAGGGACTTATGAATTGCATGTTGGGGACACCATAGACTTG
ATATTCAACTCTGAGCATCAGGTTCTAAAAAATAATACATACTGG
GGTATCATTTTACTAGCAAATCCCCAATTCATCTCCTAG.
In another non-limiting example, the APC
may express murine GITR-L as set forth in
SEQ ID NO: 97:
MEEMPLRESSPQRAERCKKSWLLCIVALLLMLLCSLGTLIYTSLK
PTAIESCMVKFELSSSKWHMTSPKPHCVNTTSDGKLKILQSGTYL
IYGQVIPVDKKYIKDNAPFVVQIYKKNDVLQTLMNDFQILPIGGV
YELHAGDNIYLKFNSKDHIQKTNTYWGII;
or as encoded by the DNA sequence set forth in
SEQ ID NO: 98:
ATGGAGGAAATGCCTTTGAGAGAATCAAGTCCTCAAAGGGCAGAG
AGGTGCAAGAAGTCATGGCTCTTGTGCATAGTGGCTCTGTTACTG
ATGTTGCTCTGTTCTTTGGGTACACTGATCTATACTTCACTCAAG
CCAACTGCCATCGAGTCCTGCATGGTTAAGTTTGAACTATCATCC
TCAAAATGGCACATGACATCTCCCAAACCTCACTGTGTGAATACG
ACATCTGATGGGAAGCTGAAGATACTGCAGAGTGGCACATATTTA
ATCTACGGCCAAGTGATTCCTGTGGATAAGAAATACATAAAAGAC
AATGCCCCCTTCGTAGTACAGATATATAAAAAGAATGATGTCCTA
CAAACTCTAATGAATGATTTTCAAATCTTGCCTATAGGAGGGGTT
TATGAACTGCATGCTGGAGATAACATATATCTGAAGTTCAACTCT
AAAGACCATATTCAGAAAACTAACACATACTGGGGGATCATCITA
ATGCCTGATCTACCATTCATCTCTTAG.
In another non-limiting example, the APC
may express human CD66a as set forth in
SEQ ID NO: 99:
MGHLSAPLHRVRVPWQGLLLTASLLTFWNPPTTAQLTTESMPFNVA
EGKEVLLLVHNLPQQLFGYSWYKGERVDGNRQIVGYAIGTQQATP
GPANSGRETIYPNASLLIQNVTQNDTGFYTLQVIKSDLVNEEATG
QFHVYPELPKPSISSNNSNPVEDKDAVAFTCEPETQDTTYLWWIN
NQSLPVSPRLQLSNGNRTLTLLSVTRNDTGPYECEIQNPVSANRS
DPVTLNVTYGPDTPTISPSDTYYRPGANLSLSCYAASNPPAQYSW
LINGTFQQSTQELFIPNITVNNSGSYTCHANNSVTGCNRTTVKTI
IVTELSPVVAKPQIKASKTTVTGDKDSVNLTCSTNDTGISIRWFF
KNQSLPSSERMKLSQGNTTLSINPVKREDAGTYWCEVFNPISKNQ
SDPIMLNVNYNALPQENGLSPGAIAGIVIGVVALVALIAVALACF
LHFGKTGSSGPLQ;
or as encoded by the DNA sequence set forth in
SEQ ID NO: 100:
ATGGGGCACCTCTCAGCCCCACTTCACAGAGTGCGTGTACCCTGG
CAGGGGCTTCTGCTCACAGCCTCACTTCTAACCTTCTGGAACCCG
CCCACCACTGCCCAGCTCACTACTGAATCCATGCCATTCAATGTT
GCAGAGGGGAAGGAGGTTCTTCTCCTTGTCCACAATCTGCCCCAG
CAACTTTTTGGCTACAGCTGGTACAAAGGGGAAAGAGTGGATGGC
AACCGTCAAATTGTAGGATATGCAATAGGAACTCAACAAGCTACC
CCAGGGCCCGCAAACAGCGGTCGAGAGACAATATACCCCAATGCA
TCCCTGCTGATCCAGAACGTCACCCAGAATGACACAGGATTCTAC
ACCCTACAAGTCATAAAGTCAGATCTTGTGAATGAAGAAGCAACT
GGACAGTTCCATGTATACCCGGAGCTGCCCAAGCCCTCCATCTCC
AGCAACAACTCCAACCCTGTGGAGGACAAGGATGCTGTGGCCTTC
ACCTGTGAACCTGAGACTCAGGACACAACCTACCTGTGGTGGATA
AACAATCAGAGCCTCCCGGTCAGTCCCAGGCTGCAGCTGTCCAAT
GGCAACAGGACCCTCACTCTACTCAGTGTCACAAGGAATGACACA
GGACCCTATGAGTGTGAAATACAGAACCCAGTGAGTGCGAACCGC
AGTGACCCAGTCACCTTGAATGTCACCTATGGCCCGGACACCCCC
ACCATTTCCCCTTCAGACACCTATTACCGTCCAGGGGCAAACCTC
AGCCTCTCCTGCTATGCAGCCTCTAACCCACCTGCACAGTACTCC
TGGCTTATCAATGGAACATTCCAGCAAAGCACACAAGAGCTCTTT
ATCCCTAACATCACTGTGAATAATAGTGGATCCTATACCTGCCAC
GCCAATAACTCAGTCACTGGCTGCAACAGGACCACAGTCAAGACG
ATCATAGTCACTGAGCTAAGTCCAGTAGTAGCAAAGCCCCAAATC
AAAGCCAGCAAGACCACAGTCACAGGAGATAAGGACTCTGTGAAC
CTGACCTGCTCCACAAATGACACTGGAATCTCCATCCGTTGGTTC
TTCAAAAACCAGAGTCTCCCGTCCTCGGAGAGGATGAAGCTGTCC
CAGGGCAACACCACCCTCAGCATAAACCCTGTCAAGAGGGAGGAT
GCTGGGACGTATTGGTGTGAGGTCTTCAACCCAATCAGTAAGAAC
CAAAGCGACCCCATCATGCTGAACGTAAACTATAATGCTCTACCA
CAAGAAAATGGCCTCTCACCTGGGGCCATTGCTGGCATTGTGATT
GGAGTAGTGGCCCTGGTTGCTCTGATAGCAGTAGCCCTGGCATGT
TTTCTGCATTTCGGGAAGACCGGCAGCTCAGGACCACTCCAATGA.
In another non-limiting example, the APC
may express murine CD66a as set forth in
SEQ ID NO: 101:
MELASAHLHKGQVPWGGLLLTASLLASWSPATTAEVTIEAVPPQV
AEDNNVLLLVHNLPLALGAFAWYKGNTTAIDKEIARFVPNSNMNF
TGQAYSGREIIYSNGSLLFQMITMKDMGVYTLDMTDENYRRTQAT
VRFHVHPILLKPNITSNNSNPVEGDDSVSLTCDSYTDPDNINYLW
SRNGESLSEGDRLKLSEGNRTLTLLNVTRNDTGPYVCETRNPVSV
NRSDPFSLNIIYGPDTPIISPSDIYLHPGSNLNLSCHAASNPPAQ
YFWLINEKPHASSQELFIPNITTNNSGTYTCFVNNSVTGLSRTTV
KNITVLEPVTQPFLQVTNTTVKELDSVTLTCLSNDIGANIQWLFN
SQSLQLTERMTLSQNNSILRIDPIKREDAGEYQCEISNPVSVRRS
NSIKLDIIFDPTQGGLSDGAIAGIVIGVVAGVALIAGLAYFLYSR
KSGGSGSF;
or as encoded by the DNA sequence set forth in
SEQ ID NO: 102:
ATGGAGCTGGCCTCAGCACATCTCCACAAAGGGCAGGTTCCCTGG
GGAGGACTACTGCTCACAGCCTCACTTTTAGCCTCCTGGAGCCCT
GCCACCACTGCTGAAGTCACCATTGAGGCTGTGCCGCCCCAGGTT
GCTGAAGACAACAATGTTCTTCTACTTGTTCACAATCTGCCCCTG
GCGCTTGGAGCCTTTGCCTGGTACAAGGGAAACACTACGGCTATA
GACAAAGAAATTGCACGATTTGTACCAAATAGTAATATGAATTTC
ACGGGGCAAGCATACAGCGGCAGAGAGATAATATACAGCAATGGA
TCCCTGCTCTTCCAAATGATCACCATGAAGGATATGGGAGTCTAC
ACACTAGATATGACAGATGAAAACTATCGTCGTACTCAGGCGACT
GTGCGATTTCATGTACACCCCATATTATTAAAGCCCAACATCACA
AGCAACAACTCCAATCCCGTGGAGGGTGACGACTCCGTATCATTA
ACCTGTGACTCTTACACTGACCCTGATAATATAAACTACCTGTGG
AGCAGAAATGGTGAAAGCCTTTCAGAAGGTGACAGGCTGAAGCTG
TCTGAGGGCAACAGGACTCTCACTTTACTCAATGTCACGAGGAAT
GACACAGGACCCTATGTGTGTGAAACCCGGAATCCAGTGAGTGTC
AACCGAAGTGACCCATTCAGCCTGAACATTATCTATGGTCCGGAC
ACCCCGATTATATCCCCCTCAGATATTTATTTGCATCCAGGGTCA
AACCTCAACCTCTCCTGCCATGCAGCCTCTAACCCACCTGCACAG
TACTTTTGGCTTATCAATGAGAAGCCCCATGCATCCTCCCAAGAG
CTCTTTATCCCCAACATCACTACTAATAATAGCGGAACCTATACC
TGCTTCGTCAATAACTCTGTCACTGGCCTCAGTAGGACCACAGTC
AAGAACATTACAGTCCTTGAGCCAGTGACTCAGCCCTTCCTCCAA
GTCACCAACACCACAGTCAAAGAACTAGACTCTGTGACCCTGACC
TGCTTGTCGAATGACATTGGAGCCAACATCCAGTGGCTCTTCAAT
AGCCAGAGTCTTCAGCTCACAGAGAGAATGACACTCTCCCAGAAC
AACAGCATCCTCAGAATAGACCCTATTAAGAGGGAAGATGCCGGC
GAGTATCAGTGTGAAATCTCGAATCCAGTCAGCGTCAGGAGGAGC
AACTCAATCAAGCTGGACATAATATTTGACCCAACACAAGGAGGC
CTCTCAGATGGCGCCATTGCTGGCATCGTGATTGGAGTTGTGGCT
GGGGTGGCTCTAATAGCAGGGCTGGCATATTTCCTCTATTCCAGG
AAGTCTGGCGGATCTGGCTCCTTCTGA.

In a particular embodiment, the APCs, in particular the B cells, have been engineered to express nucleic acids encoding OX40L (SEQ ID NO:1), 4-1BB (SEQ ID NO:5) and/or IL-12 (SEQ ID NO:49). In a particular embodiment, the APCs, in particular the B cells, have been engineered to express nucleic acids encoding at least two of OX40L (SEQ ID NO:1), 4-1BB (SEQ ID NO:5) and/or IL-12 (SEQ ID NO:49). In a particular embodiment, the APCs, in particular the B cells, have been engineered to express nucleic acids encoding OX40L (SEQ ID NO:1), 4-1BB (SEQ ID NO:5) and IL-12 (SEQ ID NO:49). In certain embodiments, the nucleic acids encoding OX40L (SEQ ID NO:1), 4-1BB (SEQ ID NO:5) and/or IL-12 (SEQ ID NO:49) are mRNAs that have been transfected into the expanded B cells prior to the contacting with the lymphocytes.

In certain embodiments, the APC culture should be at least 50% % B cells, with a detectable cytokine secretion either in the B cell culture itself or during the co-culture with leucocytes, e.g. T cells.

### 3.5 Expansion Culture and Culture Media

The lymphocyte culture is an expansion culture, i.e. selectively expanding those desired classes or subclasses of lymphocytes (preferably human lymphocytes, more preferably primary human lymphocytes, and most preferably primary human T cells (including TILs)) specific for desired antigens (e.g. express by a subject sample of tumor or infected tissue). Expansion can be performed in any suitable bioreactor known in the art or described herein, including but not limited to, GREX (Wilson Wolff), Cytiva Wave bioreactor, Ori (Ori Biotech), Cocoon (Lonza), and ADVA (ADVA Biotech). To select and harvest cells equipment such as ADVA (ADVA Biotech), LOVO (Fresenius Kabi), EKKO Millipore Sigma), Sepia (Cytiva), Elite, Miltenyi Prodigy, or similar cell selection equipment can also be used.

It is preferred herein that the method of the invention is performed in a “controlled single culture vessel”. That is, the entire expansion protocol from a patient-derived sample to the final cell population is preferably performed within a single culture vessel, without the need to transfer the culture to a larger vessel once the volume of the cell culture increases.

Within the present invention, the single culture vessel is preferably the growth chamber of a bioreactor. The growth chamber may have a shape that allows adjusting the volume of the cell culture throughout the process. In certain embodiments, the growth chamber has the shape of an inverted cone or any other shape that is tapered towards the bottom of the growth chamber. Growth chambers having such shapes allow initial culturing in relatively small volumes. At the same time, such growth chambers offer the possibility to increase the initial culture volume multifold, thereby allowing the initial cell population to expand extensively without the need to switch to a larger vessel.

It is preferred herein that the single culture vessel is “controlled”. A culture vessel is controlled if at least one parameter of the culture medium in the culture vessel can be monitored and, if necessary, adjusted. Preferably, one or more of the parameters of the culture medium that are disclosed herein can be monitored and adjusted in the controlled single culture vessel according to the invention.

Any suitable cell medium known in the art or described herein can be used for expansion. Non-limiting embodiments include commercially available media such as PRIME-XV (Irvine Scientific), X-Vivo (Lonza), Excellerate (R&D Systems), CTS Optimizer (Thermo Fisher), LymphoOne T Cell Medium (Takara), Stemline, ATCC Media (LGC Standards), and ImmunoCult TM-XF T cell expansion media. The expansion medium may contain IL-2 or a variant IL 2, which variant version, in non-limiting embodiments, includes any of the following mutations alone or in combination: M1 (Q22V, Q126A, 1129D, S130G), M2 (L18N, Q126Y, S136R, M3 Q13Y, Q126Y, 1129D, S1230R), and/or M4 (L18N, Q22V, T123A, S130R). In addition, the IL-2 variant may be any of the IL-2 variants disclosed in WO 2011/063770 or U.S. Pat. No. 8,759,486, which are fully incorporated herein by reference.

The medium can further comprise glucose from 0.5 g/l to 20 g/l, additional vitamins including MEM Vitamin mix, Glutamine, Pluronic, and one or more mitogens, including but not limited to phytohemagglutinin (PHA), concanavalin A (ConA), pokeweed mitogen (PWM), mezerein (Mzn) and/or tetradecanoyl phorbol acetate (TPA).

Preferably, the lymphocytes are cultured in an ADVA bioreactor, in particular an ADVA X3 bioreactor.

The culture medium may contain IL-2 or a variant thereof under conditions that favor the growth of lymphocytes (preferably human lymphocytes, more preferably primary human lymphocytes, and most preferably primary human T cells (including (TILs)) over tumor and other cells. In some embodiments, the IL is recombinant human IL-2 (rhIL-2). The culture medium may comprise about 5,000 IU/mL to about 9,000 IU/mL of IL-2, about 6,000 IU/mL to about 8,000 IU/mL of IL-2, or about 6,000 IU/mL to about 7,000 IU/mL of IL-2, The culture medium may comprise about 10,000 IU/mL of IL-2, about 9,000 IU/mL of IL-2, about 8,000 IU/mL of IL-2, about 7,000 IU/mL of IL-2, about 6000 IU/mL of IL-2, about 5,000 IU/mL of IL-2, about 4, 000 IU/mL, about 3,000 IU/mL of IL-2, or about 1,000 IU/mL of IL-2. Preferably, the medium is supplemented with IL-2, or an active variant thereof, throughout the entire culturing process. Preferably, IL-2, or an active variant thereof, is added to the culture medium to a final concentration of about 3000 IU/mL.

Additionally or alternatively, the culture medium may comprise human AB serum (hABs). The culture medium may comprise a final concentration of about 1% to about 20% of hABs, about 4% to about 18% of hABs, about 6% to about 15% of hABs, or about 8% to about 12% of hABs.

The culture medium may comprise about 2.5% of hABs, about 5% of hABs, about 7.5% of hABs, about 10% of hABs, about 12.5% of hABs, about 15% of hABs, about 17.5 of hABs, or about 20% of hABs. Instead of hABs, alternatives to hABs, such as human serum (huS) or platelet lysate (hPL) may be used or any synthetic hABs variants known in the art may be used.

Additionally or alternatively, the culture medium may comprise IL-15. The culture medium may comprise about 100 IU/mL to about 500 IU/mL of IL-15, about 100 IU/mL to about 400 IU/mL of IL-15, about 100 IU/mL to about 300 IU/mL of IL-15, or about 100 IU/ml to about 200 IU/mL of IL-15. The culture medium may comprise about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15.

Additionally or alternatively, the culture medium may comprise IL-21. The culture medium may comprise about 0.5 IU/mL to about 20 IU/mL of IL-21, about 0.5 IU/mL to about 15 IU/mL of IL-21, 0.5 IU/mL to about 12 IU/mL of IL-21, about 0.5 IU/mL to about 10 IU/mL of IL-21, about 0.5 IU/mL to about 5 IU/mL of IL-21, or about 0.5 IU/mL to about 1 IU/mL of IL-21. The culture medium may comprise about 20 IU/mL, about 15 IU/mL, about 12 IU/mL, about 10 IU/mL, about 5 IU/mL, about 4 IU/mL, about 3 IU/mL, about 2 IU/mL, about 1 IU/mL, or about 0.5 IU/mL of IL-21.

It is preferred herein that the APCs in the culture are genetically engineered to produce IL-12. However, instead of using genetically engineered APCs, IL-12 may also be added to the culture medium as a supplement at any suitable concentration to support expansion of lymphocytes.

The cell culture medium may also comprise one or more TNFRSF agonists. In some embodiments, the TNFRSF agonist comprises a 4-1BB agonist, which may in non-limiting examples be urelumab, utomilumab, EU-101, or a fusion protein, fragment, derivative, variant, or biosimilar thereof; the TNSFR agonist may also comprise combinations of the agonists listed herein and/or as known in the art. The TNFRSF agonist may be added at a concentration sufficient to achieve a concentration in the cell culture medium of between 0.1 μg/mL and 100 μg/mL, or between 20 μg/mL and 40 μg/mL.

It is preferred that the method of the present invention comprises the following modes:
a) Batch mode: during this step, tumor samples are co-cultured with APCs in batch mode. During this static expansion step, none or only very limited expansion of the lymphocytes takes place. Preferably, pH and dissolved oxygen (DO) concentration are monitored and controlled during the expansion initiation step and adjusted if necessary.
b) fed-batch mode: once the lymphocytes expand in the batch culture, changes in the composition of the culture medium will be observed. In particular, the concentration of glucose in the culture medium will drop and lactate will accumulate. To maintain glucose and lactate concentration within a defined range, fresh medium (containing glucose and free of lactate) is fed into the growth chamber to increase glucose concentration and to reduce the lactate concentration in the culture medium. During fed-batch mode, it is preferred that pH, DO concentration, glucose concentration and lactate concentration of the culture medium are be monitored and, if necessary, adjusted. Due to the addition of culture medium during fed-batch mode, the culture volume will increase. Fed-batch mode is preferably continued until the defined volume of the bioreactor is reached.
c) circulation mode: once the defined volume of the bioreactor is reached, the culture medium is circulated in/from the growth chamber. That is, culture medium may be removed from the growth chamber and then circulated back into the growth chamber. During circulation mode, it is preferred that pH, DO concentration, glucose concentration and lactate concentration of the culture medium are be monitored. pH and DO concentration may be adjusted to a defined value if necessary. Circulation mode is preferably performed until glucose and/or lactate concentration will be outside of a predefined acceptable range.
d) perfusion mode: once glucose and/or lactate concentration are no longer within a predefined acceptable range, the bioreactor will switch to perfusion mode. That is, growth medium is constantly or stepwise removed from the growth chamber into the waste, and fresh culture medium is added at the same time. During perfusion mode, it is preferred that pH, DO concentration, glucose concentration and lactate concentration of the culture medium are be monitored. pH and DO concentration may be adjusted to a defined value if necessary. Glucose and lactate concentration may be fine-tuned by adjusting the perfusion rate.

In a first step, tumor samples are cultured during batch mode in the growth chamber of a bioreactor for a period of 1, 2, 3, 4, 5, 6, 7, 8, 9, 10, 11 or 12 days. During this, TILs comprised in the tumor samples will migrate out of the tumor sample. However, it is to be understood that the lymphocytes may also expand at least to some degree during batch mode, for example through activation by an APC. It is preferred herein that batch mode is performed directly before the subsequent expansion steps in the same bioreactor. However, batch mode may also be omitted or shortened if the tumor sample is processed/before it is added to the bioreactor. For example, the tumor fragments may be enzymatically digested and the obtained TILs may then be transferred to a bioreactor for the expansion steps.

The batch mode is preferably performed in a batch culture, that is, no fresh culture medium is added to the cells during this step. However, it is preferred that pH and dissolved oxygen levels are regulated and monitored during batch mode and maintained in a predefined range if needed.

It is preferred that APCs and/or at least one antigen is added to the growth chamber together with the tumor samples during batch mode. However, the APCs and/or the antigens may also be added to the TILs at a later time point.

Preferably, the APCs and the antigens are added to the tumor samples in the growth chamber before the addition of an activating anti-CD3 antibody. Preferably, the APCs, and optionally the antigens, are added to the tumor samples at least 1, 2, 3, 4, 5, 6, 7, 8, 9 or 10 days before the activating anti-CD3 antibody.

In a preferred embodiment, lymphocytes are co-cultured with antigen-presenting cells (APCs), in particular with B cells. Lymphocytes and APCs may be mixed at a ratio that allows sufficient availability of MHC-presented antigenic peptides to the lymphocytes.

Further, APCs, and in particular B cells, are known to secrete cytokines that can activate T cells and thus trigger T cell expansion. As such, lymphocytes and APCs may be mixed at a ratio that allows sufficient availability of APC-secreted cytokines and co-stimulation to the lymphocytes In certain embodiments, B cells are cultured with tumor fragments that are known or suspected to contain lymphocytes, in particular TILs. In particular, it is preferred that one tumor fragment having a size of 1-3 mm³ is contacted with about 1×10⁴, 5×10⁴, 10×10⁴, 25×10⁴, 50×10⁴, 75×10⁴, 100×10⁴, 250×10⁴, 500×10⁴, 750×10⁴ or 1000×10⁴, 2500×10⁴, 5000×10⁴, 7500×10⁴, 10000×10⁴ B cells. In a particularly preferred embodiment, one tumor fragment having a size of 1-3 mm³ is contacted with about 105-107 B cells, more preferably with about 10⁶ B cells.

In certain embodiments, between 10 and 1000 tumor fragments having a size of 1-3 mm³ are added to the culture. In certain embodiments, between 25 and 500, preferably between 50 and 250, more preferably between 50 and 150, most preferably between 50 and 100 tumor fragments having a size of 1-3 mm³ are added to the culture.

Alternatively, B cells may be cultured with isolated lymphocytes, in particular isolated T cells. In certain embodiments, the T cells may be isolated from blood by any method known in the art. In certain embodiments, the T cells may be tumor-infiltrating lymphocytes that have been isolated from tumor samples, for example by enzymatic digestion of the tumor sample. In certain embodiments, the initial ratio of T cells to B cells in the culture is about 1:10000, 1:9000, 1:8000, 1:7000, 1:6000, 1:5000, 1:4000, 1:3000, 1:2000 1:1000, 1:900, 1:800, 1:700, 1:600, 1:500, 1:400, 1:300, 1:200, 1:100, 1:90, 1:80, 1:70, 1:60, 1:50, 1:40, 1:30, 1:20, 1:10, 1:9, 1:8, 1:7, 1:6, 1:5, 1:4, 1:3, 1:2 or 1:1. Preferably, the initial ratio of T cells to B cells is between 1:10000 and 1:100, more preferably between 1:3000 and 1:300.

After an initial lag period, the lymphocytes in the growth chamber start expanding in the presence of an antigen-presenting cell displaying a suitable antigen. It is preferred herein that once the lymphocytes start expanding, the composition and/or the volume of the growth medium is adjusted based on the expansion rate of the lymphocytes (transition from batch mode to fed-batch mode). For that, it is required that certain parameters of the culture medium are continuously monitored.

The batch mode is followed by a fed-batch mode, during which fresh culture medium is added to the growth chamber with the aim to adjust and/or maintain the composition of the culture medium in the growth chamber. For that, it is required that one or more parameters of the culture medium in the growth chamber are monitored and adjusted to a predefined range or value if needed. The parameters comprise, without limitation, pH, dissolved oxygen (DO) concentration, glucose concentration, lactate concentration, glutamine concentration, glutamate concentration and temperature. It is preferred herein that the concentration of glucose and lactate, and optionally glutamate and/or glutamine are adjusted by adding fresh culture medium to the growth chamber. pH and/or FO may adjusted by adjusting the oxygen and/or carbon dioxide levels in the headspace of the growth chamber. Temperature of the culture medium may be adjusted with a heating element.

When fresh culture medium is added to the growth chamber during fed-batch mode, it is preferred that the fresh culture medium is added near the bottom of the growth chamber, such that fresh medium that enters the growth chamber will be in direct contact with the lymphocytes. Preferably, the lymphocytes are separated from the inlet near the bottom of the growth chamber with a membrane or perforated barrier.

Fed-batch mode will ultimately result in an increase in culture volume. As the rate with which fresh culture medium is added to the growth chamber is dependent on the consumption of nutrients (i.e. glucose) and/or the production of metabolites (i.e. lactate), the volume of the cell culture during fed-batch mode correlates with the expansion rate of the lymphocytes. Thus, in certain embodiments, the method according to the invention comprises a step of adjusting the volume of the culture medium according to the expansion rate of the lymphocytes in the growth chamber.

In certain embodiments, the culture volume will increase during fed-batch mode at least by a factor of 2, 3, 4, 5 or 6. Preferably, fed-batch mode is performed until the maximal volume or a defined volume of the growth chamber is reached.

Once a defined cell culture volume is reached in the growth chamber, such as the maximal volume of the growth chamber, the bioreactor may be set to circulation mode. That is culture medium may be removed from the growth chamber and added back to the growth chamber. Preferably, culture medium is removed near the surface of the culture medium in the growth chamber and added back to the bottom of the growth chamber, such that a flow of culture medium will be created along the lymphocytes in the growth chamber.

During circulation mode, it is preferred that the same parameters are monitored as during fed-batch mode. Since the culture reached its final volume, no nutrients in the form of fresh media can be added. However, pH (by means of CO₂), DO (by means of O₂) and temperature (by means of a heating element) may be adjusted during circulation mode.

It has to be noted that circulation is mainly performed to reduce the consumption of fresh medium. However, the circulation mode may be omitted and instead the fed-batch mode may be directly followed by a perfusion mode.

During the final perfusion mode, medium is constantly or stepwise removed from the growth chamber and replaced with fresh medium. As for the circulation mode, used medium is preferably removed near the surface of the culture medium in the growth chamber and fresh medium is added to the bottom of the growth chamber such that it will be in contact with the lymphocytes in the growth chamber.

During perfusion mode, it is preferred that the same parameters are monitored as disclosed above for fed-batch and circulation mode. The perfusion rate may be adjusted according to the consumption of nutrients (i.e. glucose) or the formation of metabolites (i.e. lactate).

It is preferred herein that the bioreactor comprises a conditioning chamber which is connected to the growth chamber via at least one outlet. That is, culture medium can be added from the conditioning chamber into the growth chamber. Preferably, the conditioning chamber further comprises at least one inlet through which medium from the growth chamber can be pumped into the conditioning chamber. A conditioning chamber that is connected to the growth chamber via at least one inlet and at least one outlet may be used for circulating culture medium in the growth chamber.

The conditioning chamber may be used to adjust the temperature of the culture medium before it is added to the growth chamber during fed-batch mode, circulation mode and/or perfusion mode. Furthermore, on or more parameters of used culture medium may be adjusted in the conditioning chamber before the conditioned medium is added to the growth chamber.

The conditioning chamber and/or the growth chamber preferably comprises one or more sensors that allow monitoring one or more parameters of the culture medium. That is, the conditioning chamber may comprise sensors to monitor at least one parameter of the culture medium selected from: pH, dissolved oxygen (DO) concentration, glucose concentration, lactate concentration, glutamine concentration, glutamate concentration and temperature. However, the bioreactor may also comprise an analytical unit in which one or more parameters of the culture medium are determined. The analytical unit may be connected to the growth chamber such that culture medium can be transferred from the growth chamber to the analytical unit either constantly or at defined intervals, In certain embodiments, glucose and lactate concentrations, and optionally glutamate/glutamine concentrations, are measured in the analytical unit with any suitable method known in the art.

For each parameter of the culture medium, an acceptable range may be defined. It is then monitored for each individual parameter if the culture medium in the growth chamber is within the predefined acceptable range for said parameter. Certain parameters can be monitored constantly, e.g. pH, dO or temperature. However, determination of other parameters, such as glucose or lactate concentration, may be more time consuming and may thus be performed in certain intervals. For example and without limitation, certain parameters may be determined every minute, every 5 minutes, every 10 minutes, every 15 minutes, every 30 minutes or every 60 minutes.

Expansion of lymphocytes results in consumption of media components (such as glucose, glutamate or glutamine) and in the accumulation of metabolites (such as lactate or ammonium) in the culture medium. These changes in the composition of the culture medium may result in one or more parameters to no longer fall within a predefined acceptable range or to cross a predefined threshold value. If this is the case, the culture medium in the growth chamber is supplemented such that each parameter will again be within the acceptable range.

Is to be understood that the bioreactor for the process described above is equipped with at least a growth chamber which is connected to a supply of fresh media and a waste container and further comprises the necessary pumps to add fresh media to the growth chamber and to remove used media from the growth chamber.

However, it is preferred herein that the bioreactor for the process described above further comprises a conditioning chamber and the necessary pumps to circulate the culture medium between the growth chamber and the conditioning chamber. Further pumps will be required to connect the growth chamber and/or the conditioning chamber to a supply of fresh culture medium and/or to a waste container. Further, the growth chamber and/or the conditioning chamber may be equipped with the suitable sensors to monitor the parameters of the culture medium throughout the entire process. Suitable devices for the single step expansion of lymphocytes as described above are known in the art and comprise, without limitation, the ADVA X3 bioreactor. Further, a bioreactor as disclosed in WO2021/148878 may be used for the method according to the invention. WO2021/148878 is fully incorporated herein by reference.

The growth chamber is a chamber that is suitable for culturing lymphocytes, in particular T cells. It is preferred herein that the growth chamber is suitable for culturing lymphocytes by circulation and/or perfusion mode, i.e. that the growth chamber comprises at least one inlet for adding fresh or conditioned culture medium to the growth chamber and at least one outlet for removing culture medium from the growth chamber (either to a waste container or to the conditioning chamber).

Preferably, the inlet through which fresh or conditioned medium can be added to the growth chamber is located near the bottom of the growth chamber and the outlet is located at the top part of the growth chamber such that the culture medium can be removed from near the surface of the culture medium in the growth chamber. Adding culture medium to the bottom of the growth chamber and removing it from the top of the growth chamber will generate a flow of culture medium along the lymphocytes to efficiently provide them with nutrients.

In certain embodiments, the growth chamber may comprise multiple outlets in the top part of the growth chamber, wherein the outlets are arranged at different heights. Having multiple outlets at different heights allows that the growth chamber can be filled with different volumes of culture medium, while still being able to remove culture medium near the surface of the culture medium in the growth chamber.

Preferably, the cells are separated from the inlet at the bottom of the growth chamber by a perforated barrier. Growth chambers that may be used in the method of the present invention for the culturing of lymphocytes are disclosed in WO2018037402, which is fully incorporated herein by reference.

When the lymphocytes are provided with recycled, circulated culture medium, it is preferred that the bioreactor comprises a conditioning chamber in which the composition of the culture medium can be adjusted according to predefined parameters. The conditioning chamber preferably comprises one or more inlets through which the culture medium in the conditioning chamber can be supplemented. Further, the conditioning chamber may comprise one or more sensors to monitor the parameters of the culture medium in the conditioning chamber. Further, the conditioning chamber may comprise a stirrer to facilitate the mixing of the culture medium in the conditioning chamber with the supplements. To maintain the culture medium at a predefined temperature, the conditioning chamber may further comprise a heating element.

As mentioned above, the bioreactor may comprise multiple sensors to monitor the parameters in the culture medium. The sensors are preferably located in the growth chamber and/or the conditioning chamber. Alternatively or additionally, one or more sensors may also be located in the connections between the growth chamber and the conditioning chamber and/or in an analytical unit that is connected to the growth chamber and/or the conditioning chamber.

The conditioned culture medium may be based on any culture medium that is suitable for culturing lymphocytes. In particular, the conditioned growth medium may be based on any culture medium that is suitable for culturing T cells. In particular, the conditioned growth medium may be based on any T cell medium disclosed herein.

In certain embodiments, the conditioned culture medium is maintained at a defined pH range. Sensors to measure the pH of a fluid are well known in the art and are commonly used in bioreactors. The conditioned growth medium according to the invention is preferably maintained at a pH range from 6 to 8, preferably from 6.5 to 7.5, more preferably from 7.0 to 7.4. Maintaining the pH in the culture medium may be achieved by titrating the culture medium with acid or base or, more preferably, by adjusting the CO₂ concentration in the growth chamber and/or the conditioning chamber.

In certain embodiments, a defined dissolved oxygen (DO) concentration is maintained in the conditioned growth medium. Sensors or probes for measuring the dissolved oxygen concentration in a fluid are well known in the art and are commonly used in bioreactors. The conditioned growth medium according to the invention is preferably maintained at a DO concentration ranging from 10% to 100% DO, preferably from 20% to 90% DO, more preferably from 30% to 80% DO. Maintaining the DO concentration in the culture medium may be achieved by sparging air or oxygen into the culture medium.

In certain embodiments, a defined glucose concentration is maintained in the conditioned growth medium. Sensors or methods for continuously measuring the glucose concentration in a fluid are known in the art and are commonly used in bioreactors. The conditioned growth medium according to the invention is preferably maintained at a glucose concentration ranging from 0.5 to 10 g/L glucose, preferably from 1 to 8 g/L glucose, more preferably from 2 to 6 g/L glucose. Maintaining the glucose concentration in the culture medium may be achieved by adding a concentrated glucose solution to the culture medium. However, within the present invention, it is preferred that glucose concentration in the culture medium is maintained by supplementing the culture medium with fresh culture medium.

In certain embodiments, a defined glutamate concentration is maintained in the conditioned growth medium. Sensors or methods for continuously measuring the glutamate concentration in a fluid are known in the art and are commonly used in bioreactors. Maintaining the glutamate concentration in the culture medium may be achieved by adding a concentrated glutamate solution to the culture medium. However, within the present invention, it is preferred that glutamate concentration in the culture medium is maintained by supplementing the culture medium with fresh culture medium.

In certain embodiments, a defined glutamine concentration is maintained in the conditioned growth medium. Sensors or methods for continuously measuring the glutamine concentration in a fluid are known in the art and are commonly used in bioreactors. Maintaining the glutamine concentration in the culture medium may be achieved by adding a concentrated glutamine solution to the culture medium. However, within the present invention, it is preferred that glutamine concentration in the culture medium is maintained by supplementing the culture medium with fresh culture medium.

In certain embodiments, a defined lactate concentration is maintained in the conditioned growth medium. Sensors or methods for continuously measuring the lactate concentration in a fluid are known in the art and are commonly used in bioreactors. The culture medium according to the invention is preferably conditioned such that the lactate concentration is maintained below 15 mM g/L lactate, preferably 10 mM g/L lactate, more preferably 5 mM g/L lactate. Maintaining the lactate concentration in the culture medium below a defined threshold may be achieved by diluting the culture medium with fresh culture medium.

In certain embodiments, the conditioned growth medium is maintained at a defined temperature. Sensors for continuously measuring the temperature of a fluid are known in the art and are commonly used in bioreactors. The culture medium according to the invention is preferably maintained at a temperature ranging from 35 to 39° C., preferably 36 to 38° C., more preferably 36.5 to 37.5° C. Maintaining the temperature of the culture medium in a defined range may be achieved by heating means comprised within the bioreactor.

While it would be possible to supplement the growth medium in the growth chamber, it is preferred that the growth medium is supplemented in the conditioning chamber to prevent direct contact between the lymphocytes and highly concentrated supplements. However, DO and pH are preferably directly adjusted in the growth chamber by adjusting the composition of CO₂ and O₂ in the headspace of the growth chamber.

In certain embodiments, the conditioned culture medium is a medium in which at least one of the parameters pH, DO, glucose concentration, lactate concentration, glutamate concentration, glutamine concentration and/or temperature is maintained within a defined range as disclosed herein.

In certain embodiments, the conditioned culture medium is a medium in which at least two of the parameters pH, DO, glucose concentration, lactate concentration, glutamate concentration, glutamine concentration and/or temperature are maintained within a defined range as disclosed herein.

In certain embodiments, the conditioned culture medium is a medium in which at least three of the parameters pH, DO, glucose concentration, lactate concentration, glutamate concentration, glutamine concentration and/or temperature are maintained within a defined range as disclosed herein.

In certain embodiments, the conditioned culture medium is a medium in which at least four of the parameters pH, DO, glucose concentration, lactate concentration, glutamate concentration, glutamine concentration and/or temperature are maintained within a defined range as disclosed herein.

In certain embodiments, the conditioned culture medium is a medium in which at least five of the parameters pH, DO, glucose concentration, lactate concentration, glutamate concentration, glutamine concentration and/or temperature are maintained within a defined range as disclosed herein.

In certain embodiments, the conditioned culture medium is a medium in which at least six of the parameters pH, DO, glucose concentration, lactate concentration, glutamate concentration, glutamine concentration and/or temperature are maintained within a defined range as disclosed herein.

In certain embodiments, the conditioned culture medium is a medium in which all of the parameters pH, DO, glucose concentration, lactate concentration, glutamate concentration, glutamine concentration and temperature are maintained within a defined range as disclosed herein.

In certain embodiments, the conditioned culture medium is a medium in which all of the parameters pH, DO, glucose concentration, lactate concentration, and temperature are maintained within a defined range as disclosed herein.

It is to be noted that further parameters may be controlled in the conditioned growth medium. Further parameters and suitable probes/methods for determining the above-mentioned parameters are summarized in Reyes et al., Processes 2022, 10, 189. https://doi.org/10.3390/pr10020189, which is fully incorporated herein by reference.

It is to be noted that during operating the bioreactors and bioreactor systems of the present application, a liquid, e.g., a growth medium can be supplied by perfusion (constant replacement of media in and waste out), by circulation (constant replacement of media by recirculation), or by fed-batch (addition of specific nutrients to the growth medium)).

It is preferred herein that during the expansion phase, lymphocytes are perfused with conditioned culture medium. That is, during the expansion phase, conditioned culture medium is supplied to the lymphocytes while growth medium is simultaneously removed from the bioreactor. Preferably, perfusion of the lymphocytes is performed as disclosed in WO 2018/037402, which is fully incorporated herein by reference.

Expansion of lymphocytes requires the presence of an activating signal. Within the method of the present invention, it is preferred that lymphocytes are initially activated by a population of antigen presenting cells (APCs) that are co-cultured with the lymphocytes. It is preferred herein that lymphocytes are co-cultured with APCs for a period of 1, 2, 3, 4, 5, 6, 7, 8, 9, 10, 11 or 12 days. The APCs are preferably the activated B cells disclosed herein.

It is to be understood that most APCs survive in T cell medium only for a limited number of days. As such, it is preferred that an additional activator is added to the lymphocytes during the process.

In certain embodiments, the activator is an anti-CD3 antibody. Any anti-CD3 antibody that has the potential to activate lymphocytes, in particular T cells, may be used in the method of the present invention. Preferably, the anti-CD3 antibody OKT-3 is used for activating the lymphocytes in the culture.

The cell culture medium may be supplemented with an OKT-3 antibody component alone or in combination with one or more of the cytokines disclosed herein. The culture medium may comprise a final concentration of about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, or about 1 μg/mL of an OKT-3 antibody. The cell culture medium may comprise between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, or between 50 ng/mL and 100 ng/mL of OKT-3 antibody. In some embodiments, the cell culture medium does not comprise an OKT-3 antibody. In a preferred embodiment, the OKT-3 antibody is added to the culture medium to obtain a final concentration of about 100 ng/mL.

It is preferred herein that the anti-CD3 antibody, in particular the OKT-3 antibody, is added to the cell culture after the addition of the APCs. Preferably, the anti-CD3 antibody, in particular the OKT-3 antibody, is added to the culture after the lymphocytes have been cultured for at least 1, 2, 3, 4, 5, 6, 7, 8, 9, 10, 11 or 12 days in the presence of APCs. In a particularly preferred embodiment, the anti-CD3 antibody, in particular the OKT-3 antibody, is added to the culture after the lymphocytes have been cultured for 8-12 days, even more preferably for 9-11 days, most preferably for 10 days, in the presence of APCs.

In certain embodiments, lymphocytes are initially cultured together with B cells and a pool of peptides for 8-12 days, even more preferably for 9-11 days, most preferably for 10 days, before the anti-CD3 antibody, in particular the OKT-3 antibody is added to the culture.

In certain embodiments, an activator, such as an anti-CD3 antibody, may be added to the lymphocytes more than once. That is, in certain embodiments, an anti-CD3 antibody, such as OKT-3, may be added to the lymphocytes twice, wherein the second dose of the antibody is given 1, 2, 3, 4, 5, 6, 7, 8, 9 or 10 days after the first days. In certain embodiments, an anti-CD3 antibody, such as OKT-3, may be added to the lymphocytes multiple times, for example in intervals of 1, 2, 3, 4, 5, 6, 7, 8, 9 or 10 days.

The expansion phase may last from 5 to 35 days. The expansion phase may be from 5 to 30 days, from 5 to 25 days, from 5 to 20 days, or from 5 to 15 days. In certain embodiment, the expansion phase is no more than 15 days. In certain embodiments, the expansion phase may be from 25 to 50 days, from 25 to 45 days, from 25 to 40 days or from 25 to 35 days. It is further preferred that the sample comprising the lymphocytes and/or the T cells have been maintained at above 0° C. prior to expansion and are maintained throughout expansion at above 0° C.

That is, it is preferred that once obtained from the source, the sample of cells and/or the T cells subjected to expansion are not frozen at any point until the desired yield is reached, preferably at least 1×10⁷ cells. The expansion can be continued under the conditions as explained herein until at least 1×10⁷, 5×10⁷, 10×10⁷, 15×10⁷, 20×10⁷, 25×10⁷, 30×10⁷, 35×10⁷, 40×10⁷, 45×10⁷, 50×10⁷, 55×10⁷, 60×10⁷, 65×10⁷, 70×10⁷, 75×10⁷, 80×10⁷,85x 107, 90×10⁷, 95×10⁷, or at least 100×10⁷ T cells are obtained. Preferably, the expansion is continued under the conditions as explained herein until at least 10×10⁸T cells are obtained.

As described herein, the culture may also comprise feeder cells as known in the art, which may be autologous or allogenic cells such as B cells, dendritic cells, T cells, macrophages and/or PBMCs. It is also possible to replace feeder cells by cytokines in the media. Feeder cells can be added before start of the culture or any day of the expansion culture. The final yield of the expansion is preferably between 1×10⁷ and 1000×10⁷, more preferably between 10×10⁷ and 1000×10⁷ target cells (e.g. T cells). In preferred embodiments, the population after the expansion is at least 90% CD3+, comprises at least 15% cells that react to the desired antigens, e.g. neoantigens retrieved from/identified in the patients, comprises a majority of CD8+ cells, and has at least 70% viability. It is further preferred that at least half the T cells responding to a stimulation by neoantigen peptides create a durable response in the patient. For that, peripheral lymphocytes may be retrieved from the patient and tested in the presence of a neoantigen in an ELISpot assay.

Specific populations of lymphocytes can be separated from the other components of the samples and/or culture. Methods for separating a specific population of desired cells from the sample are known and include, but are not limited to, e.g. leukapheresis for obtaining T cells from the peripheral blood sample from a patient or from a donor; isolating/obtaining specific populations from the sample using a FACSort apparatus; and selecting specific populations from fresh biopsy specimens comprising living leucocytes by hand or by using a micromanipulator (see, e.g., Dudley, Immunother. 26(2003), 332-342; Robbins, Clin. Oncol. 29(20011), 917-924; Leisegang, J. Mol. Med. 86(2008), 573-58). The term “fresh biopsy specimens” refers to a tissue sample (e.g. a tumor tissue, infected tissue, or blood sample) that has been or is to be removed and/or isolated from a subject by surgical or any other known means.

As is well known in the art, it is also possible to isolate/obtain and culture/select one or more specific sub-populations of leucocytes, e.g. as most preferred T cells. Such methods include but are not limited to isolation and culture of sub-populations such as CD3+, CD28+, CD4+, CD8+, and γδ subclasses of lymphocytes, as well as the isolation and culture of other primary lymphocyte populations such as NK T cells, B cells or macrophages. Such selection methods can comprise positive and/or negative selection techniques, e.g. wherein the sample is incubated with specific combinations of antibodies and/or cytokines to select for the desired subpopulation. The skilled person can readily adjust the components of the selection medium and/or method and length of the selection using well known methods in the art. Longer incubation times may be used to isolate desired populations in any situation where there is or are expected to be fewer desired cells relative to other cell types, e.g. such as in isolating tumor-infiltrating lymphocytes (TIL) from tumor tissue or from immunocompromised individuals. The skilled person will also recognize that multiple rounds of selection can be used in the disclosed methods.

Enrichment of the desired population is also possible by negative selection, e.g. achieved with a combination of antibodies directed to surface markers unique to the negatively selected cells. In a non-limiting example, cell sorting and/or selection via negative magnetic immunoadherence or flow cytometry that uses a cocktail of monoclonal antibodies directed to cell surface markers present on the cells negatively selected can be used. For example, to enrich for CD4+ cells by negative selection, a monoclonal antibody cocktail typically including antibodies specific for, e.g. CD14, CD20, CD11b, CD16, HLA-DR, and CD8, may be used. The methods disclosed herein also encompass removing regulatory immune cells, e.g. CD25+ T cells, from the population to be expanded or otherwise included in the culture. Such methods include using an anti-CD25 antibody, or fragment thereof, or a CD25-binding ligand, such as IL-2.

The donor and/or recipient of the leucocytes and/or populations of leucocytes as disclosed herein, including the subject to be treated with the allogenic or autologous leucocytes, may be any living organism in which an immune response can be elicited (e.g., mammals). Examples of donors and/or recipients as used herein include humans, dogs, cats, mice, rats, monkeys and apes, as well as transgenic species thereof, and are preferably humans.

### 3.6 Antigens and Neoantigens

Within the present invention, it is preferred that the T cells comprised in the population of lymphocytes specifically recognize one or more predetermined antigens. This can be achieved by exposing the lymphocytes to predetermined antigens during the culturing process, which will promote expansion of T cells that specifically recognize these antigens.

As disclosed in more detail above, antigens are preferably presented to the lymphocytes by antigen-presenting cells, in particular B cells. Methods for achieving presentation of a specific antigen by an APC are disclosed herein and comprise genetic engineering of APCs or the addition of synthesized peptides to the APCs. Alternatively, homogenized tumor samples may be added to the APCs.

Neoantigens result from somatic mutations in tumor cells and are thus expressed only in tumor cells but not in normal cells. Because normal cells do not express neoantigens, they are considered non-self by the immune system. Consequently, targeting neoantigens does not easily induce autoimmunity. Thus, neoantigens are ideal targets for therapeutic cancer vaccines and T cell-based cancer immunotherapy. By taking advantage of the immune activity of neoantigens, synthetic neoantigen drugs can be designed according to the situation of tumor cell mutation to achieve the effect of treatment.

In particular embodiments, the antigens presented are neoantigens retrieved by sequencing tumors or peripheral blood cells or other potential sources of antigens of the patient to be treated (e.g. a tumor sample or sample of infected tissue) and identified by a relevant algorithm. Such algorithms are well known in the art and include, e.g. Neon (Neon Therapeutics) and Achilles (Achilles Therapeutics). The identification of neoantigens in tumor samples has been disclosed, without limitation, in WO 2017/106638, WO 2011/143656, WO 2017/011660, WO 2018/213803 or WO 2021/116714, which are fully incorporated herein by reference.

Neoantigenic peptides that may be used in the method according to the invention are disclosed in WO 2016/187508, which is fully incorporated herein by reference.

Within the method according to the invention, it is preferred that the lymphocytes, and preferably the APCs, are contacted with a pool of chemically synthesized peptides.

The pool of chemically synthesized peptides may be specifically designed for the subject that will be treated with the population of lymphocytes. For example, the pool of peptides may comprise a plurality of antigenic and/or neoantigenic peptides that are known to be associated with the specific type of cancer the subject is suffering from.

Alternatively, the pool of peptides may be personalized for the subject that is suffering from cancer. That is, the pool of peptides may comprise antigenic and/or neoantigenic peptides that have been identified to be present in the subject's tumor.

The pool of peptides may also comprise a mixture of “known” and “personalized” antigenic and/or neoantigenic peptides-

It is preferred that the pool of chemically synthesized peptides consists of or comprises neoantigenic peptides. It is further preferred that the neoantigenic peptides comprised in the pool of chemically synthesized peptides have been identified in a tumor sample of the same subject from which the lymphocytes for the culturing process have been obtained.

The identified neoantigens are peptides that can vary in length from between 6 and 20 amino acids or from 9 to 25 amino acids. Alternatively, full MHC complexes (maximum size of 45KDa) loaded with a neoantigenic peptide may be contacted with the population of cells. In certain embodiments, the invention also encompasses the use of the antigens as described herein (whether already known or identified according to the methods of the invention) to attract and retrieve peripheral immune cells (including T Cells, B Cells, NK Cells or Macrophages).

In certain embodiments the neoantigens are not individually identified, but are rather presented by adding a sample, in particular an encapsulated sample of a tumor or an infected tissue, to the lymphocyte culture.

### 3.7 Genetic Engineering

One or more cells of use in the methods disclosed herein may be genetically engineered, e.g. a lymphocyte (preferably human lymphocytes, more preferably primary human lymphocytes, and most preferably primary human T cells (including (TILs)), a feed cell and/or an APC (such as a B cell), so that it presents a desired antigen suitable to stimulate and/or activate a T cell specific for that antigen. The genetically engineered lymphocyte may transiently or stably express the encoded polypeptide. The expression can be constitutive or constitutional, depending on the system used as is known in the art. The encoding nucleic acid may or may not be stably integrated into the engineered cell's genome.

Methods for genetically engineering cells (e.g. feeder cells and/or one or more APC such as B cells) to express polypeptides of interest are known in the art and can generally be divided into physical, chemical, and biological methods. The appropriate method for given cell type and intended use can readily be determined by the skilled person using common general knowledge. Such methods for genetically engineering cells by introduction of nucleic acid molecules/sequences encoding the polypeptide of interest (e.g., in an expression vector) include but are not limited to chemical- and electroporation methods, calcium phosphate methods, cationic lipid methods, and liposome methods. The nucleic acid molecule/sequence to be transduced can be conventionally and highly efficiently transduced by using a commercially available transfection reagent and/or by any suitable method known in the art or described herein. In addition to methods of genetically engineering cells with nucleic acid molecules comprising or consisting of DNA sequences, the methods disclosed herein can also be performed with mRNA transfection. “mRNA transfection” refers to a method well known to those skilled in the art to transiently express a protein of interest.

Physical methods for introducing a polynucleotide into a host cell include calcium phosphate precipitation, lipofection, particle bombardment, microinjection, electroporation, and the like; see, e.g., Sambrook et al., 2012, Molecular Cloning: A Laboratory Manual, volumes 1-4, Cold Spring Harbor Press, NY.

Biological methods for introducing a polynucleotide of interest into a host cell include the use of DNA and RNA vectors. Viral vectors, and especially retroviral vectors, have become the most widely used method for inserting genes into mammalian cells. Accordingly, retroviral vectors are preferred for use in the methods and cells disclosed herein. Viral vectors can be derived from a variety of different viruses, including but not limited to lentivirus, poxviruses, herpes simplex virus I, adenoviruses and adeno-associated viruses; see, e.g. U.S. Pat. Nos. 5,350,674 and 5,585,362. Non-limiting examples of suitable retroviral vectors for transducing T cells include SAMEN CMV/SRa (Clay et al., J. Immunol. 163(1999), 507-513), LZRS-id3-IHRES (Heemskerk et al., J. Exp. Med. 186(1997), 1597-1602), FeLV (Neil et al., Nature 308(1984), 814-820), SAX (Kantoff et al., Proc. Natl. Acad. Sci. USA 83(1986), 6563-6567), pDOL (Desiderio, J. Exp. Med. 167(1988), 372-388), N2 (Kasid et al., Proc. Natl. Acad. Sci. USA 87(1990), 473-477), LNL6 (Tiberghien et al., Blood 84(1994), 1333-1341), pZipNEO (Chen et al., J. Immunol. 153(1994), 3630-3638), LASN (Mullen et al., Hum. Gene Ther. 7(1996), 1123-1129), pG1XsNa (Taylor et al., J. Exp. Med. 184(1996), 2031-2036), LCNX (Sun et al., Hum. Gene Ther. 8(1997), 1041-1048), SFG (Gallardo et al., Blood 90(1997), LXSN (Sun et al., Hum. Gene Ther. 8(1997), 1041-1048), SFG (Gallardo et al., Blood 90(1997), 952-957), HMB-Hb-Hu (Vieillard et al., Proc. Natl. Acad. Sci. USA 94(1997), 11595-11600), pMV7 (Cochlovius et al., Cancer Immunol. Immunother. 46(1998), 61-66), pSTITCH (Weitjens et al., Gene Ther 5(1998), 1195-1203), pLZR (Yang et al., Hum. Gene Ther. 10(1999), 123-132), pBAG (Wu et al., Hum. Gene Ther. 10(1999), 977-982), rKat.43.267bn (Gilham et al., J. Immunother. 25(2002), 139-151), pLGSN (Engels et al., Hum. Gene Ther. 14(2003), 1155-1168), pMP71 (Engels et al., Hum. Gene Ther. 14(2003), 1155-1168), pGCSAM (Morgan et al., J. Immunol. 171(2003), 3287-3295), pMSGV (Zhao et al., J. Immunol. 174(2005), 4415-4423), or pMX (de Witte et al., J. Immunol. 181(2008), 5128-5136). Most preferred are lentiviral vectors. Non-limiting examples of suitable lentiviral vectors for transducing T cells are, e.g. PL-SIN lentiviral vector (Hotta et al., Nat Methods. 6(2009), 370-376), p156RRL-sinPPT-CMV-GFP-PRE/Nhel (Campeau et al., PLoS One 4(2009), e6529), pCMVR8.74 (Addgene Catalogoue No.:22036), FUGW (Lois et al., Science 295(2002), 868-872, pLVX-EF1 (Addgene Catalogue No.: 64368), pLVE (Brunger et al., Proc Natl Acad Sci USA 111(2014), E798-806), pCDH1-MCS1-EF1 (Hu et al., Mol Cancer Res. 7(2009), 1756-1770), pSLIK (Wang et al., Nat Cell Biol. 16(2014), 345-356), pUM1 (Solomon et al., Nat Genet. 45(2013), 1428-30), pLX302 (Kang et al., Sci Signal. 6(2013), rs13), pHR-IG (Xie et al., J Cereb Blood Flow Metab. 33(2013), 1875-85), pRRLSIN (Addgene Catalogoue No.: 62053), pLS (Miyoshi et al., J Virol. 72(1998), 8150-8157), pLL3.7 (Lazebnik et al., J Biol Chem. 283(2008), 11078-82), FRIG (Raissi et al., Mol Cell Neurosci. 57(2013), 23-32), pWPT (Ritz-Laser et al., Diabetologia. 46(2003), 810-821), pBOB (Marr et al., J Mol Neurosci. 22(2004), 5-11), and pLEX (Addgene Catalogue No.: 27976).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Regardless of the method used to introduce exogenous nucleic acids into a host cell (e.g a lymphocyte (preferably human lymphocytes, more preferably primary human lymphocytes, and most preferably primary human T cells (including (TILs)), a feeder cell and/or an APC (such as a B cell)), in order to confirm the presence of the recombinant DNA sequence in the target cell (i.e., to confirm that the cell has been genetically engineered according to the methods disclosed herein), a variety of assays may be performed. Such assays include, for example, “molecular biological” assays well known to those of skill in the art, such as Southern and Northern blotting, RT-PCR and PCR; “biochemical” assays, such as detecting the presence or absence of a particular polypeptide, e.g., by immunological means (ELISAs and/or Western blots) or by assays described herein to identify whether the cell exhibits a property or activity associated with the engineered polypeptide, i.e. assays to assess whether the lymphocyte (more preferably a human primary lymphocyte such as an NK cell or T cell) exhibits CCR8 activity. Such assays are also recognized to be applicable for the testing of the expression of endogenously expressed proteins and or endogenous activity, e.g. for assessing endogenous function and/or sorting of populations based thereon.

The cells of the invention may be engineered with nucleic acid molecules to express other polypeptides suspected or known to be of use in adoptive lymphocyte therapy, e.g. with a nucleic acid sequence encoding an exogenous T cell receptor, a chimeric antigen receptor (CAR) specific for a tumor of interest, an exogenous cytokine receptor (which sequence may or may not be modified relative to the endogenous/wild-type sequence), and/or an endogenous cytokine receptor having a sequence modified relative to the wild-type sequence (i.e a modified endogenous cytokine receptor). Alternately or additionally, one or more of the T cells in the population of the invention can be further genetically modified to disrupt the expression of the endogenous T cell receptor, such that it is not expressed or expressed at a reduced level as compared to a T cell absent such modification.

As used herein, an “exogenous T cell receptor” or “exogenous TCR” refers to a TCR whose sequence is introduced into the genome of a lymphocyte (preferably human lymphocytes, more preferably primary human lymphocytes, and most preferably primary human T cells (including (TILs)) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell). Such exogenous T cell receptors can comprise alpha and beta chains or, alternatively, may comprise gamma and delta chains. Exogenous TCRs useful in the invention may have specificity to any antigen or epitope of interest.

The population of lymphocytes of the invention (preferably human lymphocytes, more preferably primary human lymphocytes, and most preferably primary human T cells (including (TILs)) may be further modified to express a chimeric antigen receptor as known in the art (also referenced as a “CAR”). Chimeric antigen receptors (CARs) are well known in the art and refer to an engineered receptor that confers or grafts specificity for an antigen onto a lymphocyte (e.g., most preferably a human primary T cell). A CAR typically comprises an extracellular ligand-binding domain or moiety and an intracellular domain that comprises one or more stimulatory domains that transduce the signals necessary for lymphocyte (e.g., T cell) activation. In some embodiments, the extracellular ligand-binding domain or moiety can be in the form of single-chain variable fragments derived from a monoclonal antibody (scFvs), which provide specificity for a particular epitope or antigen (e.g., an epitope or antigen associated with cancer, such as preferentially express on the surface of a cancer cell or other disease-causing cell). The extracellular ligand-binding domain can be specific for any antigen or epitope of interest. The intracellular stimulatory domain typically comprises the intracellular domain signaling domains of non-TCR T cell stimulatory/agonistic receptors. Such cytoplasmic signaling domains can include, for example, but not limited to, the intracellular signaling domain of CD3(, CD28, 4-1BB, OX40, or a combination thereof. A chimeric antigen receptor can further include additional structural elements, including a transmembrane domain that is attached to the extracellular ligand-binding domain via a hinge or spacer sequence.

One or more lymphocytes in the population of lymphocytes of the invention (preferably human lymphocytes, more preferably primary human lymphocytes, and most preferably primary human T cells (including (TILs)) may be genetically modified to express one or more further exogenous cytokine receptors (which may have a wild-type sequence or may have an amino acid sequence modified relative to that of the endogenous/wild-type sequence) and/or one or more endogenous cytokine receptors having a sequence modified from that of the endogenous sequence. As used herein, an “exogenous cytokine receptor” refers to a cytokine receptor whose sequence is introduced into the genome of a lymphocyte (preferably human lymphocyte, more preferably a primary human lymphocyte, and most preferably a primary human T cell (including (TIL)) that does not endogenously express the receptor. Similarly, “endogenous cytokine receptor” refers to a receptor whose sequence is introduced into the genome of such a lymphocyte that endogenously expresses the receptor. The introduced exogenous or endogenous cytokine receptor may be modified to alter the function of the receptor normally exhibited in its endogenous environment. For example, dominant-negative mutations to receptors are known that bind ligand but which ligand-receptor interaction does not elicit the endogenous activity normally associated with such interaction. Expression of an exogenous cytokine receptor (modified or not) and/or a modified endogenous receptor can confer ligand-specific activity not normally exhibited by the lymphocyte or, in the case of dominant-negative modifications, can act as ligand-sinks to bind cytokines and prevent and/or decrease the ligand-specific activity.

### 3.8 Non-Alloreactive T Cells

The population of lymphocytes obtainable by the methods described herein (preferably a human lymphocyte, more preferably a primary human lymphocyte, and most preferably a primary human T cell (such as a TIL)) are of use as a medicament, e.g., in the treatment of cancer. They and the treatment(s) based on their use may be either part of an autologous immunotherapy or part of an allogenic immunotherapy treatment. As understood in the art, “autologous” in the context of immunotherapy methods refers to the situation where the origin of the population used in the treatment is from the patient to be treated, the donor of the lymphocytes and the recipient of the immunotherapy (i.e., cell transfer) are the same. “Allogenic” in the context of immunotherapy methods refers to the situation where the origin the lymphocytes or population of lymphocytes used for the immunotherapy originate from a genetically distinct donor relative to the patient.

The populations of lymphocytes of the invention and/or obtainable by the methods disclosed herein may be genetically modified prior to, during or subsequent to expansion such that they can be used in allogenic treatments. As is known in the art, this is an effort to promote not only proper engraftment, but also to minimize undesired graft-versus-host immune reactions. In the context of the invention, such non-alloreactive engineering can be actively performed in combination with the other methods of genetic engineering herein, e.g., occurring before, concurrently with or subsequent to the methods of genetic engineering (e.g. for expression of exogenous T cell receptors and/or CARs) and/or at any time prior, during or subsequent to expansion. Accordingly, the methods of the invention may include steps of procuring a sample known or suspected to comprise lymphocytes (in particular T cells (preferably TILs) from a donor and inactivating genes thereof involved in MHC recognition as well known in the art. Such methods are generally reliant on disruption of the endogenous TCR. The TCR comprises two peptide chains, alpha and beta, which assemble to form a heterodimer that further associates with the CD3-transducing subunits to form the T cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft-versus-host immune reactions, which, when severe can present as graft-versus-host disease (GVHD). It is known that normal surface expression of the TCR depends on the coordinated synthesis and assembly of all seven components of the complex. The inactivation of TCRalpha or TCRbeta gene (and, thus, the expressed peptide) can result in the elimination of the TCR from the surface of T cells, preventing recognition of alloantigen (and, thus, GVHD) rendering the cells non-allogenic.

Alternatively, the non-alloreactive engineering methods can have been performed separately, such as to establish a universal, patient-independent source or cells, e.g., as would be available for purchase from a depository of prepared cells and which can be subsequently expanded according to the methods disclosed herein. Accordingly, the invention also encompasses the use of lymphocytes (i.e., off the shelf lymphocytes), preferably primary lymphocytes, purchased from depositories and/or that have already been engineered for the expression of one or more desirable peptides disclosed herein, e.g. engineering to express an exogenous TCR or CAR. Accordingly, the methods disclosed herein are applicable to primary lymphocytes (preferably human lymphocytes, more preferably primary human lymphocytes, and most preferably primary human T cells (including (TILs)), that are non-allogenic, i.e., “off the shelf” primary human lymphocytes.

In a similar manner the population of lymphocytes of the invention or obtainable by a method disclosed herein can be additionally or alternatively further engineered prior to, concurrently with, or subsequent to expansion to eliminate or reduce the ability to elicit an immune response, and/or to eliminate or reduce recognition by the host immune system. This is an effort to minimize or eliminate host-versus-graft immune reactions. As with the non-alloreactive engineering, the engineering of the cells to reduce or eliminate the susceptibility to the host immune system (and/or the ability to elicit a host immune reaction) can be performed before, concurrently with, or after any other engineering methods as disclosed herein. As a non-limiting exemplary embodiment, engineering the cells to reduce or eliminate the susceptibility to the host immune system (and/or the ability to elicit a host immune reaction) can be performed by reducing or eliminating expression of the endogenous major histocompatibility complex.

### 3.9 Pharmaceutical Compositions

In a particular embodiment, the invention relates to a pharmaceutical composition comprising the population of lymphocytes according to the invention.

The population of lymphocytes of the invention is intended for use in adoptive cell transfer (ACT) therapy in humans. That is, the cells comprised in the population of lymphocytes are preferably suspended in a liquid that is suitable for injection into the human bodies. Suitable liquids for suspending the cells comprised in the population of lymphocytes include, without limitation, pharmaceutically acceptable buffers.

In certain embodiments, the pharmaceutically acceptable buffer may be a sodium chloride buffer. In certain embodiments, the pharmaceutically acceptable buffer may be a 0.9% NaCl buffer. In certain embodiments, the pharmaceutically acceptable buffer may be supplemented with at least 5%, 10%, 15% or 20% DMSO to allow freezing of the population of lymphocytes. In certain embodiments, the pharmaceutically acceptable buffer may comprise between 0 and 15% DMSO. That is, the pharmaceutically acceptable buffer may comprise 0.9% NaCl and 0%, 1%, 2%, 3%, 4%, 5%, 6%, 7%, 8%, 9%, 10%, 11%, 12%, 13%, 14% or 15% DMSO.

It is preferred that the pharmaceutical composition is substantially free of bacterial contaminants, in particular mycoplasma. The absence of bacteria/mycoplasma can be tested with devices or kits known in the art such as, without limitation, with a BacTec device and/or a MycoSeq kit. Further, it is preferred that the pharmaceutical composition is substantially free of endotoxins.

The term “medicament” is used interchangeably with the term “pharmaceutical composition” and relates to a composition suitable for administration to a patient, preferably a human patient. Accordingly, the invention provides a population of lymphocytes (preferably human lymphocytes, more preferably primary human lymphocytes, and most preferably primary human T cells (including (TILs)—which may or may not be further genetically engineered to express one or more desired peptides or receptors) for use as a medicament and methods of producing such populations of lymphocytes for such use. The medicament/pharmaceutical composition may be administered to an allogenic recipient, i.e. to recipient that is a different individual from that donating the T cells, or to an autologous recipient, i.e. wherein the recipient patient also donated the T cells. Alternately the medicament/pharmaceutical composition may comprise non-allogenic lymphocytes, (“off the shelf” lymphocytes as known in the art). Regardless of the species of the patient, the donor and recipient (patient) are of the same species. It is preferred that the patient/recipient is a human.

In the manufacture of a pharmaceutical formulation according to the invention, the expanded population of lymphocytes (preferably human lymphocytes, more preferably primary human lymphocytes, and most preferably primary human T cells (including (TILs)) are typically admixed with a pharmaceutically acceptable carrier excipient and/or diluent and the resulting composition is administered to a subject. The carrier must, of course, be acceptable in the sense of being compatible with any other ingredients in the formulation and must not be deleterious to the subject or engineered cells. Examples of suitable pharmaceutical carriers are well known in the art and include phosphate buffered saline solutions, water, emulsions, such as oil/water emulsions, various types of wetting agents, sterile solutions etc. The carrier may be a solution that is isotonic with the blood of the recipient. Compositions comprising such carriers can be formulated by well-known conventional methods. The pharmaceutical compositions of the invention can further comprise one or more additional agents useful in the treatment of a disease in the subject. The pharmaceutical compositions of the invention can further include biological molecules known to be advantageous to lymphocyte function or activity, including but not limited to cytokines (e.g. IL-2, IL-7, IL-15, and/or IL-21), which promote in vivo cell proliferation and engraftment. The population of lymphocytes of the invention can be administered in the same composition as the one or more additional agent or biological molecule or, alternatively, can be co-administered in separate compositions.

The pharmaceutical compositions described herein can be used in combination with a chemotherapeutic agent. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General chemotherapeutic agents considered for use in combination therapies include anastrozole, bicalutamide, bleomycin sulfate, busulfan, capecitabine, N4-pentoxycarbonyl-5-deoxy-5-fluorocytidine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, Gemcitabine, hydroxyurea (Hydrea®), Idarubicin, ifosfamide, irinotecan, L-asparaginase, leucovorin calcium, melphalan, 6-mercaptopurine, methotrexate, mitoxantrone, mylotarg, paclitaxel, Yttrium90/MX-DTPA, pentostatin, tamoxifen citrate, teniposide, 6-thioguanine, thiotepa, tirapazamine, topotecan hydrochloride, vinblastine, vincristine, and vinorelbine.

Anti-cancer agents for use in combination with the populations of lymphocytes of the invention include but are not limited to, anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatin, pemetrexed, raltitrexed, cladribine, clofarabine, azacitidine, decitabine and gemcitabine.

Exemplary alkylating agents include, without limitation, nitrogen mustards, uracil mustard, ethylenimine derivatives, alkyl sulfonates, nitrosoureas, triazenes, chlormethine, cyclophosphamide, ifosfamide, melphalan, chlorambucil, pipobroman, triethylenemelamine, triethylenethiophosphoramine, temozolomide, thiotepa, busulfan, carmustine, lomustine, streptozocin, dacarbazine, oxaliplatin, temozolomide, dactinomycin, melphalan, altretamine, carmustine, bendamustine, busulfan, carboplatin, lomustine, cisplatin, chlorambucil, cyclophosphamide, dacarbazine, altretamine, ifosfamide, prednumustine, procarbazine, mechlorethamine, streptozocin, thiotepa, cyclophosphamide, and bendamustine HCl.

### 3.10 Therapeutic Applications

The populations of the lymphocytes of the invention or obtainable by the methods disclosed herein (preferably a population of human lymphocytes, more preferably primary human lymphocytes, and most preferably primary human T cells (including (TILs)) are envisioned as for use as a medicament in the treatment of diseases including, but not limited to, cancers or precancerous conditions. The term “cancer” or “proliferative disease” as used herein means any disease, condition, trait, genotype or phenotype characterized by unregulated cell growth or replication as is known in the art. Because the characteristic feature of the cancer/proliferative disease or precancerous condition is irrelevant to the methods disclosed herein, i.e. the population of lymphocytes is specifically expanded to be selective for the desired antigens, e.g. neoantigens of the specific cancer, the cancers/proliferative diseases that can be treated according to the methods and with the populations of lymphocytes disclosed herein include all types of tumors, lymphomas, and carcinomas.

Non-limiting examples of such cancers include colorectal cancer, brain cancer, ovarian cancer, prostate cancer, pancreatic cancer, breast cancer, renal cancer, nasopharyngeal carcinoma, hepatocellular carcinoma, melanoma, skin cancer, oral cancer, head and neck cancer, esophageal cancer, gastric cancer, cervical cancer, bladder cancer, lymphoma, chronic or acute leukemia (such as B, T, and myeloid derived), sarcoma, lung cancer and multidrug resistant cancer.

The terms “treatment”, “treating” and the like are used herein to generally mean obtaining a desired pharmacological and/or physiological effect. The effect may be prophylactic in terms of completely or partially preventing a disease or symptom thereof, and/or may be therapeutic in terms of partially or completely curing the disease or condition, and/or adverse effect attributed to the disease or condition. The term “treatment” as used herein covers any treatment of a disease or condition in a subject and includes: (a) preventing and/or ameliorating a proliferative disease (preferably cancer) from occurring in a subject that may be predisposed to the disease; (b) inhibiting the disease, i.e., arresting its development, such as inhibition of cancer progression; (c) relieving the disease, i.e. causing regression of the disease, such as the repression of cancer; and/or (d) preventing, inhibiting or relieving any symptom or adverse effect associated with the disease or condition. Preferably, the term “treatment” as used herein relates to medical intervention of an already manifested disorder, e.g., the treatment of a diagnosed cancer.

The treatment or therapy (i.e., comprising the use of a medicament/pharmaceutical composition comprising a population of lymphocytes disclosed herein or obtainable by the methods disclosed herein) may be administered alone or in combination with appropriate treatment protocols for the particular disease or condition as known in the art. Non-limiting examples of such protocols include but are not limited to, administration of pain medications, administration of chemotherapeutics, therapeutic radiation, and surgical handling of the disease, condition or symptom thereof. Accordingly the treatment regimens disclosed herein encompass the administration of the population of lymphocytes as disclosed herein or obtainable by the methods disclosed herein together with none, one, or more than one treatment protocol suitable for the treatment or prevention of a disease, condition or a symptom thereof, either as described herein or as known in the art. Administration “in combination” or the use “together” with other known therapies encompasses the administration of the medicament/pharmaceutical composition of the invention before, during, after or concurrently with any of the co-therapies disclosed herein or known in the art.

The pharmaceutical composition/medicament disclosed herein can be administered alone or in combination with other therapies or treatments during periods of active disease, or during a period of remission or less active disease.

When administered in combination, the population of lymphocytes of the invention or obtainable with a method of the invention, can be administered in an amount or dose that is higher, lower or the same than the amount or dosage where each therapy or agent would be used individually, e.g., as a monotherapy. In certain embodiments, the administered amount or dosage of the lymphocyte therapy, and/or at least one additional agent or therapy is lower (e.g., at least 20%, at least 30%, at least 40%, or at least 50%) than the amount or dosage of the corresponding therapy(ies) or agent(s) used individually.

The population of lymphocytes of the invention and/or obtainable by a method disclosed herein may further be rendered resistant to chemotherapy drugs that are used as standards of care as described herein or known in the art. Engineering such resistance into the populations of lymphocytes of the invention is expected to help the selection and expansion of such engineered lymphocytes in vivo in patients undergoing chemotherapy or immunosuppression.

The population of lymphocytes of the invention and/or obtainable by a method disclosed herein may undergo robust in vivo T cell expansion upon administration to a patient, and may remain persist in the body fluids for an extended amount of time, preferably for a week, more preferably for 2 weeks, even more preferably for at least one month. The population of lymphocytes of the invention and/or obtainable by a method disclosed herein may also be additionally engineered with safety switches that allow for potential control of the cell therapeutics. Such safety switches of potential use in cell therapies are known in the art and include (but are not limited to) the engineering of the cells to express targets allowing antibody depletion (e.g., truncated EGFR; Paszkiewicz et al., J Clin Invest 126(2016), 4262-4272), introduction of artificial targets for small molecule inhibitors (e.g., HSV-TK; Liang et al., Nature 563(2018), 701-704) and introduction of inducible cell death genes (e.g., icaspase; Minagawa et al., Methods Mol Biol 1895(2019), 57-73).

The administration of the population of lymphocytes according to the present invention may be carried out in any convenient manner, including by aerosol inhalation, injection, ingestion, transfusion, implantation or transplantation. The medicaments and compositions described herein may be administered subcutaneously, intradermaly, intratumorally, intranodally, intramedullary, intramuscularly, by intravenous or intralymphatic injection, or intraperitoneally. The lymphocytes, medicament and/or compositions of the present invention are preferably administered by intravenous injection.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. For example, the population of lymphocytes of the invention and/or obtainable by a method disclosed herein may be administered to the subject at a dose of 10⁴ to 10¹⁰ T cells/kg body weight, preferably 10⁵ to 10⁶ T cells/kg body weight. In the context of the present invention the lymphocytes may be administered in such a way that an upscaling of the T cells to be administered is performed by starting with a subject dose of about 10⁵ to 10⁶ T cells/kg body weight and then increasing to dose of 10¹⁰ T cells/kg body weight. The cells or population of cells can be administrated in one or more doses.

In a particular embodiment, the invention relates to a method for treating cancer, the method comprising the steps of:
a) providing a population of lymphocytes according to the invention or a pharmaceutical composition according to the invention; and
b) infusing the population of lymphocytes or the pharmaceutical composition into a subject suffering from cancer.

It is preferred herein that the population of lymphocytes or the pharmaceutical composition according to the invention is used in autologous cell therapy, in particular for the treatment of cancer. That is, it is preferred herein that the lymphocytes comprised in the population of lymphocytes or the pharmaceutical composition according to the invention are obtained by expanding a sample of lymphocytes that has been obtained from a subject suffering from cancer. Subsequently, the population of lymphocytes, preferably in the form of a pharmaceutical composition, may be infused back into the same subject.

When used in autologous cell therapy, it is preferred that the lymphocytes in the composition of lymphocytes specifically attack the subject's tumor. For that, it is required that at least part of the lymphocytes in the population of lymphocytes recognize an antigen that is present in the subject's tumor. To ensure that at least part of the lymphocytes in the population of lymphocytes recognize an antigen that is present in the subject's tumor, it is preferred that the lymphocytes are expanded in the presence of an antigenic peptide that has previously been identified as being present in the subject's tumor.

That is, in a particular embodiment the invention relates to a method for treating cancer in a subject, the method comprising the steps of:
a) surgically removing a tumor from a subject or taking a biopsy from a subject's tumor;
b) identifying at least one tumor antigen in the tumor sample obtained in step (a);
c) expanding lymphocytes in the tumor sample obtained in step (a) with the method according to the invention, wherein the lymphocytes are expanded in the presence of at least antigen that has been identified in step (b) to be present in the tumor sample;
d) infusing the expanded lymphocytes into the subject from which the tumor sample has been obtained.

The term “tumor antigen” as used throughout this specification refers to an antigen that is uniquely or differentially expressed by a tumor cell, whether intracellular or on the tumor cell surface (preferably on the tumor cell surface), compared to a normal or non-neoplastic cell. By means of example, a tumor antigen may be present in or on a tumor cell and not typically in or on normal cells or non-neoplastic cells (e.g., only expressed by a restricted number of normal tissues, such as testis and/or placenta), or a tumor antigen may be present in or on a tumor cell in greater amounts than in or on normal or non-neoplastic cells, or a tumor antigen may be present in or on tumor cells in a different form than that found in or on normal or non-neoplastic cells. The term thus includes tumor-specific antigens (TSA), including tumor-specific membrane antigens, tumor-associated antigens (TAA), including tumor-associated membrane antigens, embryonic antigens on tumors, growth factor receptors, growth factor ligands, etc. The term further includes cancer/testis (CT) antigens.

Examples of tumor antigens include, without limitation, β-human chorionic gonadotropin (DHCG), glycoprotein 100 (gp100/Pmel17), carcinoembryonic antigen (CEA), tyrosinase, tyrosinase-related protein 1 (gp75/TRP-1), tyrosinase-related protein 2 (TRP-2), NY-BR-1, NY-CO-58, NY-ESO-1, MN/gp250, idiotypes, telomerase, synovial sarcoma X breakpoint 2 (SSX2), mucin 1(MUC1), antigens of the melanoma-associated antigen (MAGE) family, high molecular weight melanoma-associated antigen (HMW-MAA), melanoma antigen recognized by T cells 1 (MART1), Wilms' tumor gene 1 (WT1), HER2/neu, mesothelin (MSLN), alphafetoprotein (AFP), cancer antigen 125 (CA-125), and abnormal forms of ras or p53 (see also, WO2016187508A2). Tumor antigens may also be subject specific (e.g., subject specific neoantigens; see, e.g., U.S. Pat. No. 9,115,402; and international patent application publication numbers WO 2016/100977, WO 2014/168874, WO 2015/085233, and WO 2015/095811)

In a preferred embodiment, the population of lymphocytes for use in the treatment of cancer comprises Neo-TILs. Neo-TILs are tumor-infiltrating lymphocytes, preferably T cells, which specifically recognize a neoantigen. Neo-TILs may be specifically expanded by contacting tumor samples or T cells obtained from tumor samples with a neoantigenic peptide as described in more detail herein. It is preferred that the presence of the neoantigen has been confirmed in the patient which receives the population of lymphocytes comprising the Neo-TILs.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

### 4. EXAMPLES

### 4.1 Preparation of B Cells

B cells are obtained from frozen apheresis sample. After thawing, the apheresis sample is washed and B cells are isolated using a commercial B cell isolation kit. The isolated B cells are then activated by adding IL-4 (final concentration: 200 IU/ml) and CD40L (final concentration: 1 μg/ml).

Subsequent to the activation step and prior to the contacting with the T lymphocytes, B cell are transfected with mRNAs encoding 4-1BB, OX40L and IL-12. For that, B cells and mRNAs are mixed and cells are transfected using an electroporation device and a suitable electroporation buffer.

Electroporated B cells are resuspended in medium supplemented with 200 μg/mL Pen-Strep and 10% human AB serum (hABS). Resuspended B cells are stored or directly used as antigen-presenting cells (APCs) for the expansion of T lymphocytes.

The aim is to prepare 100×10⁶ B cells in a volume of 40 mL.

### 4.2 Preparation of Tumor Samples

Tumor specimens (fresh or cryopreserved) are cut into small fragments (1-3 mm³). The aim is to prepare 60 tumor fragments in 50 mL of the supplemented medium.

Alternatively, tumor samples are dissociated with a commercial kit (including a step of enzymatic digestion of the tumor samples) and the obtained lymphocytes are prepared in supplemented media.

### 4.3 Preparation of Peptide Solution

A stock solution of chemically synthesized peptides (peptide library comprising 2 to 100 different peptides having a length of 9 to 25 amino acids) is prepared. Aimed peptide stock concentration is 100 μg/mL is dissolved in 20% DMSO.

### 4.4 Expansion of T Lymphocytes

60 tumor fragments or equivalent and electroporated B cells are seeded within appropriate media into the ADVA bioreactor (ADVA biotechnology).
100×10⁶ B cells in 40 mL medium supplemented with 200 μg/mL Pen-Strep and 10% human AB serum (hABS) (see section 4.1).
60 tumor fragments (1-2 mm³) in 50 mL medium supplemented with 200 μg/mL Pen-Strep, 10% hABS and 6000 iU/mL IL-2 (see Section 4.2).

B cells and tumor fragments are cultured in batch mode in ADVA X3 bioreactor for 1 day. (pH and dO are monitored and CO₂/O₂ are adjusted in the headspace of the growth chamber if necessary. After 24 h peptides are added to ADVA X3 bioreactor.

Batch mode is continued while pH, dO, glucose and lactate concentrations are monitored. Culture volume is increased by adding fresh medium to keep the four parameters within range

### Day 10: Activate Lymphocytes (+/−5 Days)

Add 15 mL activation medium comprising the anti-CD3 antibody OKT3 to obtain a final OKT3 concentration of 100 ng/mL in the culture.

Subsequently, add IL-2 every 3 days to keep the IL-2 concentration high.

Continue increasing culture media based on pH, DO, Glucose and Lactate concentration. Based on process parameters, switch from fed-batch to circulation mode and finally to perfusion mode.

Harvest the cells with the ADVA X3, exchange media and prepare cells for final formulation. Formulated cells are distributed/aliquoted and frozen for storage until analysis.

1. A method for expansion of a population of lymphocytes specific for one or more antigens in a controlled single culture vessel, the method comprising:
a) culturing a tissue or blood sample from a subject in the presence of said one or more antigens, wherein said tissue or blood sample is known or suspected to contain lymphocytes; or
b) culturing lymphocytes in the presence of said one or more antigens, wherein said lymphocytes are isolated from a tissue or blood sample from a subject;
wherein the lymphocytes are cultured in a conditioned culture medium.

2. The method according to claim 1, wherein the conditioned culture medium is a culture medium in which at least one, two, three, four or all of culture medium parameters is/are monitored and adjusted if necessary, said culture medium parameters comprising: pH, dissolved oxygen (DO) concentration, glucose concentration, lactate concentration and/or temperature.

3. (canceled)

4. The method according to claim 1, the expansion of the lymphocytes exhibits an expansion rate, and wherein the method further comprises adjusting volume of the conditioned culture medium according to the expansion rate of the lymphocytes.

5. The method according to claim 4, wherein the conditioned culture medium volume increases at least by a factor of 2, 3, 4, 5 or 6 during the expansion of the lymphocytes.

6. The method according to claim 1, the method further comprising dynamic culturing the lymphocytes with the conditioned culture medium.

7. The method according to claim 1, wherein
the tissue sample is derived from a tumor, in particular wherein the tissue sample is a tumor sample, optionally, wherein the tumor and/or tumor sample comprises at least one neoantigen.

8. (canceled)

9. The method according to claim 1, wherein the lymphocytes comprise tumor-infiltrating lymphocytes, in particular wherein the tumor-infiltrating lymphocytes are T cells.

10. The method according to claim 1, wherein one or more antigens are added to the conditioned culture medium in the form of peptides, optionally, wherein the peptides are added to the conditioned culture medium at a concentration of 0.1 to 10 μg/ml.

11. (canceled)

12. The method according to claim 1, wherein said culturing of (b) comprises a step of co-culturing the lymphocytes with antigen-presenting cells (APCs), optionally, wherein the antigen-presenting cells (APCs) are engineered to present one or more antigens.

13. (canceled)

14. The method according to claim 12, wherein the antigen-presenting cells (APCs) comprise or are B cells, optionally, wherein the B cells are obtained by apheresis.

15. (canceled)

16. The method according to claim 14, wherein the B cells are activated before addition to the lymphocytes, optionally, wherein the B cells are activated with IL-4 and/or CD40L.

17. (canceled)

18. The method according to claim 12, wherein the antigen-presenting cells (APCs) are genetically engineered to express one or more transgene, optionally, wherein the genetically engineered APCs have been obtained by transfecting the APCs with nucleic acids encoding the one or more transgene, optionally, wherein at least one of the one or more transgene encodes an immunomodulator.

19. (canceled)

20. (canceled)

21. The method according to claim 18, wherein the immunomodulator is selected from the group consisting of: OX40L, 4-1BBL, CD80, CD86, CD83, CD70, CD40L, GITR-L, CD127L, CD30L (CD153), LIGHT, BTLA, ICOS-L (CD275), SLAM (CD150), CD662L, interleukin-12, interleukin-7, interleukin-15, interleukin-17, interleukin-21, interleukin-4, Bcl6, BCLXL, BCL-2, MCL1, STAT-5, and activators of one or more signaling pathways, comprising one or more of JAK/STAT pathway, Akt/PKB signaling pathway, BCR signaling pathway, and/or AFF/BAFFR signaling pathway), optionally, wherein the immunomodulator is one or more of OX40L, 4-1BB and/or interleukin 12.

22. (canceled)

23. The method according to claim 7, wherein the method comprises confirming the presence of at least one of the one or more antigens in the tumor sample comprising the lymphocytes prior to the culturing step, and/or wherein at least one of the one or more antigens is a neoantigen and wherein method comprises confirming the presence of said neoantigen in the tumor sample comprising the lymphocytes prior to the culturing step.

24. (canceled)

25. The method according to claim 23, wherein confirming the presence of at least one of the one or more antigens in the tumor sample comprises sequencing genomic DNA that has been obtained from the tumor sample.

26. The method according to claim 1, wherein the method further comprises activating the lymphocytes during culturing, optionally, wherein activating of lymphocytes comprises addition of a CD3 agonist to the conditioned culture medium, optionally, wherein the CD3 agonist is added to the conditioned culture medium after 1, 2, 3, 4, 5, 6, 7, 8, 9, 10, 11 or 12 days.

27. (canceled)

28. (canceled)

29. The method according to claim 1, wherein the conditioned culture medium is supplemented with human AB serum and/or IL-2.

30. The method according to claim 9, wherein said culturing is continued until said T cells reaches at least 10⁷ cells.

31. The method according to claim 1, wherein said culturing is performed at temperatures of greater than 0° C.

32. The method according to claim 1, wherein said sample or said lymphocytes are maintained at temperatures greater than 0° C. subsequent to isolation from said subject and prior to said culture.

33. A population of lymphocytes obtainable by the method of claim 1.

34. A population of lymphocytes comprising at least 90% CD3+ T cells and less than 5% B cells, wherein at least 70% of said T cells are viable, at least 20% are CD27/CD28 double positive and less than 10% are triple positive for CD45RA, CD57 and KLRG1.

35. The population of lymphocytes according to claim 34, wherein said T cells are specific for one or more antigens.

36. The population of lymphocytes according to claim 34, wherein less than 15% of said T cells secrete at least one protein from the group consisting of: TNF-α, IL-4 and IL-5, and/or wherein at least 50%, at least 60%, at least 70%, at least 80% or at least 90% of the T cells are CD8+ T cells.

37. (canceled)

38. The population of lymphocytes according to claim 34, wherein at least two T cells of said T cells are directed against different antigens, optionally, wherein at least one antigen is a neoantigen.

39. (canceled)

40. The population of lymphocytes according to claim 34, wherein said T cells comprises at least 10⁷ T cells.

41. A pharmaceutical composition comprising the population of lymphocytes according to claim 33.

42. The pharmaceutical composition according to claim 41, wherein the lymphocytes are suspended in a pharmacologically acceptable buffer.

43. The pharmaceutical composition according to claim 42, wherein the pharmaceutically acceptable buffer comprises about 0.9% NaCl and, optionally, up to 15% DMSO.

44. (canceled)

45. (canceled)

46. (canceled)

47. (canceled)

48. A method of treating cancer, the method comprising:
a) providing a population of lymphocytes according to claim 33; and
b) infusing the population of lymphocytes into a subject suffering from cancer.

49. A method of treating cancer in a subject, the method comprising:
a) obtaining a tumor sample by surgically removing a tumor from a subject or taking a biopsy from a subject's tumor, wherein the tumor sample comprises lymphocytes;
b) identifying at least one tumor antigen in the tumor sample obtained in step (a);
c) expanding the lymphocytes comprised in the tumor sample obtained in step (a) with the method according to claim 1, wherein the lymphocytes are expanded in the presence of the at least one tumor antigen that has been identified in step (b) to be present in the tumor sample; and
d) infusing the expanded lymphocytes obtained in step (c) into the subject from which the tumor sample has been obtained.

50. The method according to claim 49, wherein the at least one tumor antigen is a tumor-associated antigen or a tumor-specific antigen.

51. The method according to claim 48, wherein the lymphocytes comprise tumor-infiltrating lymphocytes (TILs), optionally, wherein the TILs specifically recognize one or more tumor antigens, optionally, wherein at least one tumor antigen is a neoantigen.

52. (canceled)

53. (canceled)

54. The method according to claim 49, wherein the lymphocytes comprise tumor-infiltrating lymphocytes (TILs), optionally, wherein the TILs specifically recognize one or more tumor antigens, optionally, wherein the at least one tumor antigen is a neoantigen.


SEQUENCE LISTING (102 sequences)

SEQ ID NO: 1 - protein, 133 aa, Homo sapiens
  OX40L
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - DNA, 402 bp, Homo sapiens
  OX40L
atggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 3 - protein, 197 aa, Mus musculus
  OX40L
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDHFREDHNPISIPMLNDGRRIVFTVVASLAFKDKVYLTVNAPDTLCEHLQINDGELIVVQLTPGYCAPEGSYHSTVNQVPL

SEQ ID NO: 4 - DNA, 597 bp, Mus musculus
  OX40L
atggaaggggaaggggttcaacccctggatgagaatctggaaaacggatcaaggccaagattcaagtggaagaagacgctaaggctggtggtctctgggatcaagggagcagggatgcttctgtgcttcatctatgtctgcctgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtga

SEQ ID NO: 5 - protein, 254 aa, Homo sapiens
  4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - DNA, 765 bp, Homo sapiens
  4-1BBL
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 7 - protein, 309 aa, Mus musculus
  4-1BBL
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 8 - DNA, 930 bp, Mus musculus
  4-1BBL
atggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatga

SEQ ID NO: 9 - protein, 285 aa, Homo sapiens
  CD80
MEVPPPAPRSFLCRALCLFPRVFAAEAVTADSEVLEERQKRLPYVPEPYYPESGWDRLRELFGKDEQQRISKDLANICKTAATAGIIGWVYGGIPAFIHAKQQYIEQSQAEIYHNRFDAVQSAHRAATRGFIRYGWRWGWRTAVFVTIFNTVNTSLNVYRNKDALSHFVIAGAVTGSLFRINVGLRGLVAGGIIGALLGTPVGGLLMAFQKYSGETVQERKQKDRKALHELKLEEWKGRLQVTEHLPEKIESSLQEDEPENDAKKIEALLNLPRNPSVIDKQDKD

SEQ ID NO: 10 - DNA, 858 bp, Homo sapiens
  CD80
atggaggtgccgccaccggcaccgcggagctttctctgtagagcattgtgcctatttccccgagtctttgctgccgaagctgtgactgccgattcggaagtccttgaggagcgtcagaagcggcttccctacgtcccagagccctattacccggaatctggatgggaccgcctccgggagctgtttggcaaagatgaacagcagagaatttcaaaggaccttgctaatatctgtaagacggcagctacagcaggcatcattggctgggtgtatgggggaataccagcttttattcatgctaaacaacaatacattgagcagagccaggcagaaatttatcataaccggtttgatgctgtgcaatctgcacatcgtgctgccacacgaggcttcattcgttatggctggcgctggggttggagaactgcagtgtttgtgactatattcaacacagtgaacactagtctgaatgtataccgaaataaagatgccttaagccattttgtaattgcaggagctgtcacgggaagtctttttaggataaacgtaggcctgcgtggcctggtggctggtggcataattggagccttgctgggcactcctgtaggaggcctgctgatggcatttcagaagtactctggtgagactgttcaggaaagaaaacagaaggatcgaaaggcactccatgagctaaaactggaagagtggaaaggcagactacaagttactgagcacctccctgagaaaattgaaagtagtttacaggaagatgaacctgagaatgatgctaagaaaattgaagcactgctaaaccttcctagaaacccttcagtaatagataaacaagacaaggactga

SEQ ID NO: 11 - protein, 306 aa, Mus musculus
  CD80
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 12 - DNA, 918 bp, Mus musculus
  CD80
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctt

SEQ ID NO: 13 - protein, 247 aa, Homo sapiens
  CD86
MGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 14 - DNA, 744 bp, Homo sapiens
  CD86
atgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtgttatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaa

SEQ ID NO: 15 - protein, 309 aa, Mus musculus
  CD86
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAE

SEQ ID NO: 16 - DNA, 930 bp, Mus musculus
  CD86
atggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggagattacagcttcagttactgtggccctcctccttgtgatgctgctcatcattgtatgtcacaagaagccgaatcagcctagcaggcccagcaacacagcctctaagttagagcgggatagtaacgctgacagagagactatcaacctgaaggaacttgaaccccaaattgcttcagcaaaaccaaatgcagagtga

SEQ ID NO: 17 - protein, 146 aa, Homo sapiens
  CD83
METPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELV

SEQ ID NO: 18 - DNA, 441 bp, Homo sapiens
  CD83
atggagacaccccaggaagaccacctcaggggacagcactatcatcagaaggggcaaaatggttctttcgacgcccccaatgaaaggccctattccctgaagatccgaaacactaccagctgcaactcggggacatacaggtgcactctgcaggacccggatgggcagagaaacctaagtggcaaggtgatcttgagagtgacaggatgccctgcacagcgtaaagaagagacttttaagaaatacagagcggagattgtcctgctgctggctctggttattttctacttaacactcatcattttcacttgtaagtttgcacggctacagagtatcttcccagatttttctaaagctggcatggaacgagcttttctcccagttacctccccaaataagcatttagggctagtgactcctcacaagacagaactggtatga

SEQ ID NO: 19 - protein, 196 aa, Mus musculus
  CD83
MSQGLQLLFLGCACSLAPAMAMREVTVACSETADLPCTAPWDPQLSYAVSWAKVSESGTESVELPESKQNSSFEAPRRRAYSLTIQNTTICSSGTYRCALQELGGQRNLSGTVVLKVTGCPKEATESTFRKYRAEAVLLFSLVVFYLTLIIFTCKFARLQSIFPDISKPGTEQAFLPVTSPSKHLGPVTLPKTETV

SEQ ID NO: 20 - DNA, 591 bp, Mus musculus
  CD83
atgtcgcaaggcctccagctcctgtttctaggctgcgcctgcagcctggcacccgcgatggcgatgcgggaggtgacggtggcttgctccgagaccgccgacttgccttgcacagcgccctgggacccgcagctctcctatgcagtgtcctgggccaaggtctccgagagtggcactgagagtgtggagctcccggagagcaagcaaaacagctccttcgaggcccccaggagaagggcctattccctgacgatccaaaacactaccatctgcagctcgggcacctacaggtgtgccctgcaggagctcggagggcagcgcaacttgagcggcaccgtggttctgaaggtgacaggatgccccaaggaagctacagagtcaactttcaggaagtacagggcagaagctgtgttgctcttctctctggttgttttctacctgacactcatcattttcacctgcaaatttgcacgactacaaagcattttcccagatatttctaaacctggtacggaacaagcttttcttccagtcacctccccaagcaaacatttggggccagtgacccttcctaagacagaaacggtatga

SEQ ID NO: 21 - protein, 208 aa, Homo sapiens
  CD70
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGLFGFWNWGLKVKCFLRHLIWTAHCFIPLTQLVFMQALQSWRNHHCSHFTDEENRGVNR

SEQ ID NO: 22 - DNA, 627 bp, Homo sapiens
  CD70
atgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaagggctttttggattttggaactggggactcaaagtcaagtgcttcttacggcatttaatatggactgcacactgttttatcccattaactcagctcgtgttcatgcaagccctacaaagctggaggaatcatcattgttcccatttcacagatgaggaaaacagaggcgtaaaccgttga

SEQ ID NO: 23 - protein, 195 aa, Mus musculus
  CD70
MPEEGRPCPWVRWSGTAFQRQWPWLLLVVFITVFCCWFHCSGLLSKQQQRLLEHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICP

SEQ ID NO: 24 - DNA, 588 bp, Mus musculus
  CD70
atgccggaggaaggtcgcccttgcccctgggttcgctggagcgggaccgcgttccagcgccaatggccatggctgctgctggtggtgtttattactgtgttttgctgttggtttcattgtagcggactactcagtaagcagcaacagaggctgctggagcaccctgagccgcacacagctgagttacagctgaatctcacagttcctcggaaggaccccacactgcgctggggagcaggcccagccttgggaaggtccttcacacacggaccagagctggaggagggccatctgcgtatccatcaagatggcctctacaggctgcatatccaggtgacactggccaactgctcttccccaggcagcaccctgcagcacagggccaccctggctgtgggcatctgctcccccgctgcgcacggcatcagcttgctgcgtgggcgctttggacaggactgtacagtggcattacagcgcctgacatacctggtccacggagatgtcctctgtaccaacctcaccctgcctctgctgccgtcccgcaacgctgatgagaccttctttggagttcagtggatatgcccttga

SEQ ID NO: 25 - protein, 261 aa, Homo sapiens
  IL7/CD127
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGLSLSYGPVSPIIRRLWNIFVRNQEK

SEQ ID NO: 26 - DNA, 786 bp, Homo sapiens
  IL7/CD127
atgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggattaagcctatcgtatggcccagtctccccgatcataagaagactctggaacatctttgtaagaaaccaagaaaagtga

SEQ ID NO: 27 - protein, 286 aa, Mus musculus
  IL7/CD127
MMALGRAFAIVFCLIQAVSGESGNAQDGDLEDADADDHSFWCHSQLEVDGSQHLLTCAFNDSDINTANLEFQICGALLRVKCLTLNKLQDIYFIKTSEFLLIGSSNICVKLGQKNLTCKNMAINTIVKAEAPSDLKVVYRKEANDFLVTFNAPHLKKKYLKKVKHDVAYRPARGESNWTHVSLFHTRTTIPQRKLRPKAMYEIKVRSIPHNDYFKGFWSEWSPSSTFETPEPKNQGGWDPVLPSVTILSLFSVFLLVILAHVLWKKRIKPVVWPSLPDHKKTLEQL

SEQ ID NO: 28 - DNA, 861 bp, Mus musculus
  IL7/CD127
atgatggctctgggtagagctttcgctatagttttctgcttaattcaagctgtttctggagaaagtggaaatgcccaggatggagacctagaagatgcagacgcggacgatcactccttctggtgccacagccagttggaagtggatggaagtcaacatttattgacttgtgcttttaatgactcagacatcaacacagctaatctggaatttcaaatatgtggggctcttttacgagtgaaatgcctaactcttaacaagctgcaagatatatattttataaagacatcagaattcttactgattggtagcagcaatatatgtgtgaagcttggacaaaagaatttaacttgcaaaaatatggctataaacacaatagttaaagccgaggctccctctgacctgaaagtcgtttatcgcaaagaagcaaatgattttttggtgacatttaatgcacctcacttgaaaaagaaatatttaaaaaaagtaaagcatgatgtggcctaccgcccagcaaggggtgaaagcaactggacgcatgtatctttattccacacaagaacaacaatcccacagagaaaactacgaccaaaagcaatgtatgaaatcaaagtccgatccattccccataacgattacttcaaaggcttctggagcgagtggagtccaagttctaccttcgaaactccagaacccaagaatcaaggaggatgggatcctgtcttgccaagtgtcaccattctgagtttgttctctgtgtttttgttggtcatcttagcccatgtgctatggaaaaaaaggattaaacctgtcgtatggcctagtctccccgatcataagaaaactctggaacaactatag

SEQ ID NO: 29 - protein, 164 aa, Homo sapiens
  CD30L
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPDYCGMILHHSHSTLDSGKGHCCLETLQP

SEQ ID NO: 30 - DNA, 495 bp, Homo sapiens
  CD30L
atggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagggctccattcaagaagtcatgggcctacctccaagtggcaaagcatctaaacaaaaccaagttgtcttggaacaaagatggcattctccatggagtcagatatcaggatgggaatctggtgatccaattccctgattactgtggcatgatcctccaccattcacactctaccctggactctgggaagggacactgctgccttgaaactctacaaccctga

SEQ ID NO: 31 - protein, 220 aa, Mus musculus
  CD30L
MEPGLQQAGSCGAPSPDPAMQVQPGSVASPWRSTRPWRSTSRSYFYLSTTALVCLVVAVAIILVLVVQKKDSTPNTTEKAPLKGGNCSEDLFCTLKSTPSKKSWAYLQVSKHLNNTKLSWNEDGTIHGLIYQDGNLIVQFPGLYFIVCQLQFLVQCSNHSVDLTLQLLINSKIKKQTLVTVCESGVQSKNIYQNLSQFLLHYLQVNSTISVRVDNFQYVD

SEQ ID NO: 32 - DNA, 720 bp, Mus musculus
  CD30L
atggagccagggctgcaacaagcaggcagctgtggggctccttcccctgacccagccatgcaggtgcagcccggctcggtagccagcccctggagaagcacgaggccctggagaagcacaagtcgcagctacttctacctcagcaccaccgcactggtgtgccttgttgtggcagtggcgatcattctggtactggtagtccagaaaaaggactccactccaaatacaactgagaaggccccccttaaaggaggaaattgctcagaggatctcttctgtaccctgaaaagtactccatccaagaagtcatgggcctacctccaagtgtcaaagcatctcaacaataccaaactgtcatggaacgaagatggcaccatccacggactcatataccaggacgggaacctgatagtccaattccctggcttgtacttcatcgtttgccaactgcagttcctcgtgcagtgctcaaatcattctgtggacctgacattgcagctcctcatcaattccaagatcaaaaagcagacgttggtaacagtgtgtgagtctggagttcagagtaagaacatctaccagaatctctctcagtttttgctgcattacttacaggtcaactctaccatatcagtcagggtggataatttccagtatgtggatacaaacactttccctcttgataatgtgctatccgtcttcttatatagtagctcagactga

SEQ ID NO: 33 - protein, 231 aa, Homo sapiens
  LIGHT
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPR

SEQ ID NO: 34 - DNA, 694 bp, Homo sapiens
  LIGHT
atggagcctcctggagactgggggcctcctccctggagatccacccccaaaaccgacgtcttgaggctggtgctgtatctcaccttcctgggagccccctgctacgccccagctctgccgtcctgcaaggaggacgagtacccagtgggctccgagtgctgccccaagtgcagtccaggttatcgtgtgaaggaggcctgcggggagctgacgggcacagtgtgtgaaccctgccctccaggcacctacattgcccacctcaatggcctaagcaagtgtctgcagtgccaaatgtgtgacccagccatgggcctgcgcgcgagccggaactgctccaggacagagaacgccgtgtgtggctgcagcccaggccacttctgcatcgtccaggacggggaccactgcgccgcgtgccgcgcttacgccacctccagcccgggccagagggtgcagaagggaggcaccgagagtcaggacaccctgtgtcagaactgccccccggggaccttctctcccaatgggaccctggaggaatgtcagcaccagaccaagtgcagctggctggtgacgaaggccggagctgggaccagcagctcccactgggtatggtggtttctctcagggagcctcgtcatcgtcattgtttgctccacagttggcctaatcatatgtgtgaaaagaagaaagccaaggg

SEQ ID NO: 35 - protein, 239 aa, Mus musculus
  LIGHT
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 36 - DNA, 720 bp, Mus musculus
  LIGHT
atggagagtgtggtacagccttcagtgtttgtggtggatggacagacggacatcccattcaggcggctggaacagaaccaccggagacggcgctgtggcactgtccaggtcagcctggccctggtgctgctgctaggtgctgggctggccactcagggctggtttctcctgagactgcatcaacgtcttggagacatagtagctcatctgccagatggaggcaaaggctcctgggagaagctgatacaagatcaacgatctcaccaggccaacccagcagcacatcttacaggagccaacgccagcttgataggtattggtggacctctgttatgggagacacgacttggcctggccttcttgaggggcttgacgtatcatgatggggccctggtgaccatggagcccggttactactatgtgtactccaaagtgcagctgagcggcgtgggctgcccccaggggctggccaatggcctccccatcacccatggactatacaagcgcacatcccgctacccgaaggagttagaactgctggtcagtcggcggtcaccctgtggccgggccaacagctcccgagtctggtgggacagcagcttcctgggcggcgtggtacatctggaggctggggaagaggtggtggtccgcgtgcctggaaaccgcctggtcagaccacgtgacggcaccaggtcctatttcggagctttcatggtctga

SEQ ID NO: 37 - protein, 289 aa, Homo sapiens
  BTLA
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 38 - DNA, 870 bp, Homo sapiens
  BTLA
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccagtgcttcctaatgacaatgggtcataccgctgttctgcaaattttcagtctaatctcattgaaagccactcaacaactctttatgtgacagatgtaaaaagtgcctcagaacgaccctccaaggacgaaatggcaagcagaccctggctcctgtatcgtttacttcctttggggggattgcctctactcatcactacctgtttctgcctgttctgctgcctgagaaggcaccaaggaaagcaaaatgaactctctgacacagcaggaagggaaattaacctggttgatgctcaccttaagagtgagcaaacagaagcaagcaccaggcaaaattcccaagtactgctatcagaaactggaatttatgataatgaccctgacctttgtttcaggatgcaggaagggtctgaagtttattctaatccatgcctggaagaaaacaaaccaggcattgtttatgcttccctgaaccattctgtcattggaccgaactcaagactggcaagaaatgtaaaagaagcaccaacagaatatgcatccatatgtgtgaggagttaa

SEQ ID NO: 39 - protein, 305 aa, Mus musculus
  BTLA
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLIISDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 40 - DNA, 918 bp, Mus musculus
  BTLA
atgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataatatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaa

SEQ ID NO: 41 - protein, 309 aa, Homo sapiens
  ICOS-L
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTESWNLLLLLS

SEQ ID NO: 42 - DNA, 930 bp, Homo sapiens
  ICOS-L
atgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactgaatcctggaacctgctccttctgctctcgtga

SEQ ID NO: 43 - protein, 343 aa, Mus musculus
  ICOS-L
CPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTDTWAPVPYQDYLIPRYLMSPCLKTRGLP

SEQ ID NO: 44 - DNA, 1033 bp, Mus musculus
  ICOS-L
gtgtccctgttttgtgtccttgggaaccaggcagcctgtttggaagaagctccatgtttctagcgggttcttttctggtcttggtctgttcttgctgctgttgagcagcctctgtgctgcctctgcagagactgaagtcggtgcaatggtgggcagcaatgtggtgctcagctgcattgacccccacagacgccatttcaacttgagtggtctgtatgtctattggcaaatcgaaaacccagaagtttcggtgacttactacctgccttacaagtctccagggatcaatgtggacagttcctacaagaacaggggccatctgtccctggactccatgaagcagggtaacttctctctgtacctgaagaatgtcacccctcaggatacccaggagttcacatgccgggtatttatgaatacagccacagagttagtcaagatcttggaagaggtggtcaggctgcgtgtggcagcaaacttcagtacacctgtcatcagcacctctgatagctccaacccgggccaggaacgtacctacacctgcatgtccaagaatggctacccagagcccaacctgtattggatcaacacaacggacaatagcctaatagacacggctctgcagaataacactgtctacttgaacaagttgggcctgtatgatgtaatcagcacattaaggctcccttggacatctcgtggggatgttctgtgctgcgtagagaatgtggctctccaccagaacatcactagcattagccaggcagaaagtttcactggaaataacacaaagaacccacaggaaacccacaataatgagttaaaagtccttgtccccgtccttgctgtactggcggcagcggcattcgtttccttcatcatatacagacgcacgcgtccccaccgaagctatacaggacccaagactgtacagcttgaacttacagacacttgggctcccgtcccctaccaggactatttgattccaagatatttgatgtctccatgcctcaaaacacgtggtttaccataa

SEQ ID NO: 45 - protein, 335 aa, Homo sapiens
  CD150
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 46 - DNA, 1008 bp, Homo sapiens
  CD150
atggatcccaaggggctcctctccttgaccttcgtgctgtttctctccctggcttttggggcaagctacggaacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatgagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcagaaacaaaaccatgggcagtgtatgctgggctgttagggggtgtcatcatgattctcatcatggtggtaatactacagttgagaagaagaggtaaaacgaaccattaccagacaacagtggaaaaaaaaagccttacgatctatgcccaagtccagaaaccaggtcctcttcagaagaaacttgactccttcccagctcaggacccttgcaccaccatatatgttgctgccacagagcctgtcccagagtctgtccaggaaacaaattccatcacagtctatgctagtgtgacacttccagagagctga

SEQ ID NO: 47 - protein, 343 aa, Mus musculus
  CD150
MDPKGSLSWRILLFLSLAFELSYGTGGGVMDCPVILQKLGQDTWLPLTNEHQINKSVNKSVRILVTMATSPGSKSNKKIVSFDLSKGSYPDHLEDGYHFQSKNLSLKILGNRRESEGWYLVSVEENVSVQQFCKQLKLYEQVSPPEIKVLNKTQENENGTCSLLLACTVKKGDHVTYSWSDEAGTHLLSRANRSHLLHITLSNQHQDSIYNCTASNPVSSISRTFNLSSQACKQESSSESSPWMQYTLVPLGVVIIFILVFTAIIMMKRQGKSNHCQPPVEEKSLTIYAQVQKSGPQEKKLHDALTDQDPCTTIYVAATEPAPESVQEPNPTTVYASVTLPES

SEQ ID NO: 48 - DNA, 1032 bp, Mus musculus
  CD150
atggatcccaaaggatccctttcctggagaatacttctgtttctctccctggcttttgagttgagctacggaacaggtggaggtgtgatggattgcccagtgattctccagaagctgggacaggacacgtggctgcccctgacgaatgaacatcagataaataagagcgtgaacaaaagtgtccgcatcctcgtcaccatggcgacgtccccaggaagcaaatccaacaagaaaattgtgtcttttgatctctctaaagggagctatccagatcacctggaggatggctaccactttcaatcaaaaaacctgagcctgaagatcctcgggaacaggcgggagagtgaaggatggtacttggtgagcgtggaggagaacgtttctgttcagcaattctgcaagcagctgaagctttatgaacaggtctcccctccagagattaaagtgctaaacaaaacccaggagaacgagaatgggacctgcagcttgctgttggcctgcacagtgaagaaaggggaccatgtgacttacagctggagtgatgaggcaggcacccacctgctgagccgagccaaccgctcccacctcctgcacatcactcttagcaaccagcatcaagacagcatctacaactgcaccgcaagcaaccctgtcagcagtatctctaggaccttcaacctatcatcgcaagcatgcaagcaggaatcctcctcagaatcgagtccatggatgcaatatactcttgtaccactgggggtcgttataatcttcatcctggttttcacggcaataataatgatgaaaagacaaggtaaatcaaatcactgccagccaccagtggaagaaaaaagccttactatttatgcccaagtacagaaatcagggcctcaagagaagaaacttcatgatgccctaacagatcaggacccctgcacaaccatttatgtggctgccacagagcctgccccagagtctgtccaggaaccaaaccccaccacagtttatgccagtgtgacactgccagagagctga

SEQ ID NO: 49 - protein, 215 aa, Homo sapiens
  IL-12
MWPPGSASQPPPSPAAATGLHPAARPVSLQCRLSMCPARSLLLVATLVLLDHLSLARNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 50 - DNA, 648 bp, Homo sapiens
  IL-12
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 51 - protein, 335 aa, Mus musculus
  IL-12
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 52 - DNA, 1008 bp, Mus musculus
  IL-12
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 53 - protein, 133 aa, Homo sapiens
  IL-7
MFHVSFRYIFGLPPLILVLLPVASSDCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 54 - DNA, 401 bp, Homo sapiens
  IL-7
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactg

SEQ ID NO: 55 - protein, 154 aa, Mus musculus
  IL-7
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 56 - DNA, 465 bp, Mus musculus
  IL-7
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataa

SEQ ID NO: 57 - protein, 162 aa, Homo sapiens
  IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 58 - DNA, 489 bp, Homo sapiens
  IL-15
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 59 - protein, 180 aa, Homo sapiens
  IL-17
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 60 - DNA, 543 bp, Homo sapiens
  IL-17
atggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctga

SEQ ID NO: 61 - protein, 158 aa, Mus musculus
  IL-17
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 62 - DNA, 477 bp, Mus musculus
  IL-17
atgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaa

SEQ ID NO: 63 - protein, 162 aa, Homo sapiens
  IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 64 - DNA, 489 bp, Homo sapiens
  IL-21
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 65 - protein, 146 aa, Mus musculus [sequence omitted]

SEQ ID NO: 66 - DNA, 441 bp, Mus musculus
  IL-21
atggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctag

SEQ ID NO: 67 - protein, 447 aa, Homo sapiens
  IL-1
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEGMCVMEQSKGLLL

SEQ ID NO: 68 - DNA, 1344 bp, Homo sapiens
  IL-1
atgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaaggtatgtgtgtaatggaacagagtaaaggcttattgttgtaa

SEQ ID NO: 69 - protein, 576 aa, Mus musculus
  IL-1
MENMKVLLGLICLMVPLLSLEIDVCTEYPNQIVLFLSVNEIDIRKCPLTPNKMHGDTIIWYKNDSKTPISADRDSRIHQQNEHLWFVPAKVEDSGYYYCIVRNSTYCLKTKVTVTVLENDPGLCYSTQATFPQRLHIAGDGSLVCPYVSYFKDENNELPEVQWYKNCKPLLLDNVSFFGVKDKLLVRNVAEEHRGDYICRMSYTFRGKQYPVTRVIQFITIDENKRDRPVILSPRNETIEADPGSMIQLICNVTGQFSDLVYWKWNGSEIEWNDPFLAEDYQFVEHPSTKRKYTLITTLNISEVKSQFYRYPFICVVKNTNIFESAHVQLIYPVPDFKNYLIGGFIILTATIVCCVCIYKVFKVDIVLWYRDSCSGFLPSKASDGKTYDAYILYPKTLGEGSFSDLDTFVFKLLPEVLEGQFGYKLFIYGRDDYVGEDTIEVTNENVKKSRRLIIILVRDMGGFSWLGQSSEEQIAIYNALIQEGIKIVLLELEKIQDYEKMPDSIQFIKQKHGVICWSGDFQERPQSAKTRFWKNLRYQMPAQRRSPLSKHRLLTLDPVRDTKEKLPAATHLPLG

SEQ ID NO: 70 - DNA, 1731 bp, Mus musculus
  IL-1
atggagaatatgaaagtgctactggggctcatttgtctcatggtgcctctgctgtcgctggagattgacgtatgtacagaatatccaaatcagatcgttttgtttttatctgtaaatgaaattgatattcgcaagtgtcctcttactccaaataaaatgcacggcgacaccataatttggtacaagaatgacagcaagacccccatatcagcggaccgggactccaggattcatcagcagaatgaacatctttggtttgtacctgccaaggtggaggactcaggatattactattgtatagtaagaaactcaacttactgcctcaaaactaaagtaaccgtaactgtgttagagaatgaccctggcttgtgttacagcacacaggccaccttcccacagcggctccacattgccggggatggaagtcttgtgtgcccttatgtgagttattttaaagatgaaaataatgagttacccgaggtccagtggtataagaactgtaaacctctgcttcttgacaacgtgagcttcttcggagtaaaagataaactgttggtgaggaatgtggctgaagagcacagaggggactatatatgccgtatgtcctatacgttccgggggaagcaatatccggtcacacgagtaatacaatttatcacaatagatgaaaacaagagggacagacctgttatcctgagccctcggaatgagacgatcgaagctgacccaggatcaatgatacaactgatctgcaacgtcacgggccagttctcagaccttgtctactggaagtggaatggatcagaaattgaatggaatgatccatttctagctgaagactatcaatttgtggaacatccttcaaccaaaagaaaatacacactcattacaacacttaacatttcagaagttaaaagccagttttatcgctatccgtttatctgtgttgttaagaacacaaatatttttgagtcggcgcatgtgcagttaatatacccagtccctgacttcaagaattacctcatcgggggctttatcatcctcacggctacaattgtatgctgtgtgtgcatctataaagtcttcaaggttgacatagtgctttggtacagggactcctgctctggttttcttccttcaaaagcttcagatggaaagacatacgatgcctatattctttatcccaagaccctgggagaggggtccttctcagacttagatacttttgtttttaaactgttgcctgaggtcttggagggacagtttggatacaagctgttcatttatggaagggatgactatgttggagaagataccatcgaggttactaatgaaaatgtaaagaaaagcaggaggctgattatcattctagtgagagatatgggaggcttcagctggctgggccagtcatctgaagagcaaatagccatatacaatgctctcatccaggaaggaattaaaatcgtcctgcttgagttggagaaaatccaagactatgagaaaatgccagattctattcagttcattaagcagaaacacggagtcatttgctggtcaggagactttcaagaaagaccacagtctgcaaagaccaggttctggaaaaacttaagataccagatgccagcccaacggagatcaccattgtctaaacaccgcttactaaccctggatcctgtgcgggacactaaggagaaactgccggcagcaacacacttaccactcggctag

SEQ ID NO: 71 - protein, 479 aa, Homo sapiens
  BCL-6
MGSPAAPEGALGYVREFTRHSSDVLGNLNELRLRGILTDVTLLVGGQPLRAHKAVLIACSGFFYSIFRGRAGVGVDVLSLPGGPEARGFAPLLDFMYTSRLRLSPATAPAVLAAATYLQMEHVVQACHRFIQASYEPLGISLRPLEAEPPTPPTAPPPGSPRRSEGHPDPPTESRSCSQGPPSPASPDPKACNWKKYKYIVLNSQASQAGSLVGERSSGQPCPQARLPSGDEASSSSSSSSSSSEEGPIPGPQSRLSPTAATVQFKCGAPASTPYLLTSQAQDTSGSPSERARPLPGSEFFSCQNCEAVAGCSSGLDSLVPGDEDKPYKCQLCRSSFRYKGNLASHRTVHTGEKPYHCSICGARFNRPANLKTHSRIHSGEKPYKCETCGSRFVQVAHLRAHVLIHTGEKPYPCPTCGTRFRHLQTLKSHVRIHTGEKPYHCDPCGLHFRHKSQLRLHLRQKHGAATNTKVHYHILGGP

SEQ ID NO: 72 - DNA, 1440 bp, Homo sapiens
  BCL-6
atgggttcccccgccgccccggagggagcgctgggctacgtccgcgagttcactcgccactcctccgacgtgctgggcaacctcaacgagctgcgcctgcgcgggatcctcactgacgtcacgctgctggttggcgggcaacccctcagagcacacaaggcagttctcatcgcctgcagtggcttcttctattcaattttccggggccgtgcgggagtcggggtggacgtgctctctctgcccgggggtcccgaagcgagaggcttcgcccctctattggacttcatgtacacttcgcgcctgcgcctctctccagccactgcaccagcagtcctagcggccgccacctatttgcagatggagcacgtggtccaggcatgccaccgcttcatccaggccagctatgaacctctgggcatctccctgcgccccctggaagcagaacccccaacacccccaacggcccctccaccaggtagtcccaggcgctccgaaggacacccagacccacctactgaatctcgaagctgcagtcaaggcccccccagtccagccagccctgaccccaaggcctgcaactggaaaaagtacaagtacatcgtgctaaactctcaggcctcccaagcagggagcctggtcggggagagaagttctggtcaaccttgcccccaagccaggctccccagtggagacgaggcctccagcagcagcagcagcagcagcagcagcagtgaagaaggacccattcctggtccccagagcaggctctctccaactgctgccactgtgcagttcaaatgtggggctccagccagtaccccctacctcctcacatcccaggctcaagacacctctggatcaccctctgaacgggctcgtccactaccgggaagtgaatttttcagctgccagaactgtgaggctgtggcagggtgctcatcggggctggactccttggttcctggggacgaagacaaaccctataagtgtcagctgtgccggtcttcgttccgctacaagggcaaccttgccagtcatcgtacagtgcacacaggggaaaagccttaccactgctcaatctgcggagcccgttttaaccggccagcaaacctgaaaacgcacagccgcatccattcgggagagaagccgtataagtgtgagacgtgcggctcgcgctttgtacaggtggcacatctgcgggcgcacgtgctgatccacaccggggagaagccctacccttgccctacctgcggaacccgcttccgccacctgcagaccctcaagagccacgttcgcatccacaccggagagaagccttaccactgcgacccctgtggcctgcatttccggcacaagagtcaactgcggctgcatctgcgccagaaacacggagctgctaccaacaccaaagtgcactaccacattctcggggggccctag

SEQ ID NO: 73 - protein, 707 aa, Mus musculus
  BCL-6
MASPADSCIQFTRHASDVLLNLNRLRSRDILTDVVIVVSREQFRAHKTVLMACSGLFYSIFTDQLKCNLSVINLDPEISPEGFCILLDFMYTSRLNLREGNIMAVMTTAMYLQMEHVVDTCRKFIKASEAEMAPALKPPREEFLNSRMLMPHDIMAYRGREVVENNMPLRNTPGCESRAFAPPLYSGLSTPPASYPMYSHLPLSTFLFSDEELRDAPRMPVANPFPKERALPCDSARQVPNEYSRPAMEVSPSLCHSNIYSPKEAVPEEARSDIHYSVPEGPKPAVPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGSEQAELGRLSPRAYPAPPACQPPMEPANLDLQSPTKLSASGEDSTIPQASRLNNLVNRSLAGSPRSSSESHSPLYMHPPKCTSCGSQSPQHTEMCLHTAGPTFPEEMGETQSEYSDSSCENGTFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSAADLPPELPKAC

SEQ ID NO: 74 - DNA, 2124 bp, Mus musculus
  BCL-6
atggcctccccggctgacagctgtatccagtttacccggcacgctagtgatgttcttctcaaccttaatcgcctccggagtcgggacatcttgacggacgttgtcatcgtggtgagccgtgagcagtttagagcccataagacagtgctcatggcctgcagcggcctgttctacagtatcttcactgaccagttgaaatgcaaccttagtgtaatcaatctagatcctgaaatcagccctgaggggttttgcatcctcctggacttcatgtacacatctaggctcaacctgagggaaggcaatatcatggcggtgatgaccacagccatgtacctgcagatggagcatgttgtcgacacatgcaggaagttcatcaaggccagtgaagcagaaatggcccctgcacttaaacctccccgtgaagagttcctgaacagccggatgctgatgccccatgacatcatggcctaccgaggtcgtgaggtcgtggagaacaatatgccactgagaaatactcccgggtgtgagagcagagcttttgctcctcctctgtacagtggcctgtcaacaccaccagcctcttatcccatgtacagccatctcccgctcagcaccttcctcttctctgatgaggagctccgagatgccccccgaatgcctgtggccaacccttttcccaaggagcgtgccctcccctgcgacagtgccaggcaagtccctaatgagtatagcaggccagccatggaggtgtcccccagtttgtgtcacagcaacatctactcgcccaaggaggcagtcccagaggaggctcggagtgacatacactacagtgtgcctgagggccccaagcctgctgtcccttctgctcggaatgctccatacttcccctgtgacaaagccagcaaagaagaagagagaccttcttcggaggatgagattgccctgcatttcgagccccccaatgcacccttgaaccggaagggtctggttagtccccagagtccccagaaatccgactgccagcccaactcacccacagagtcctgcagcagcaagaacgcctgcatccttcaggcctctggctctccgccagccaagagccccactgacccgaaagcctgcaactggaagaagtataagttcatcgttctcaacagcctcaatcagaatgccaaacccgagggctctgagcaggcagagctgggtcgcctctcccctcgagcctaccctgcaccgcccgcttgccagccgcctatggagcccgcgaaccttgatctccagtccccgaccaagctcagtgccagtggggaggactctaccatcccccaagccagccggctcaataatctcgtgaacaggtccctggcaggctccccccgaagcagcagtgagagtcactcaccactctacatgcaccccccaaagtgcacatcctgcggctctcagtccccacagcatacagagatgtgcctccatactgctgggcccacgttcccggaggagatgggggaaacccagtcagagtattcggattctagctgtgagaatgggaccttcttctgcaacgaatgtgactgccgtttctctgaggaggcctcgctcaagaggcacacgctgcagacgcacagtgacaaaccatacaaatgtgatcgctgccaggcctccttccgctacaagggcaacctcgccagccacaagactgtccacacgggtgagaaaccctatcgctgtaacatttgtggagcgcagttcaatcggccagccaacctgaagacccacactcgaattcactctggagaaaagccctacaaatgtgaaacctgtggggccaggtttgttcaggtggcccacctccgtgcccacgtgctcatccacactggagagaagccgtacccctgtgaaatctgtggcactcgcttccggcaccttcagactctgaagagccatctgcgcatccacacaggagagaaaccttaccattgtgagaagtgtaacctgcactttcgtcacaaaagccaactgcgacttcatttgcgccagaagcacggcgccatcaccaacaccaaggtgcaataccgcgtgtcggccgctgacctgcctccggagctccccaaagcctgctga

SEQ ID NO: 75 - protein, 233 aa, Homo sapiens
  BCLXL
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 76 - DNA, 702 bp, Homo sapiens
  BCLXL
atgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 77 - protein, 233 aa, Mus musculus
  BCLXL
MSQSNRELVVDFLSYKLSQKGYSWSQFSDVEENRTEAPEETEAERETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIASWMATYLNDHLEPWIQENGGWDTFVDLYGNNAAAESRKGQERFNRWFLTGMTVAGVVLLGSLFSRK

SEQ ID NO: 78 - DNA, 702 bp, Mus musculus
  BCLXL
atgtctcagagcaaccgggagctggtggtcgactttctctcctacaagctttcccagaaaggatacagctggagtcagtttagtgatgtcgaagagaataggactgaggccccagaagaaactgaagcagagagggagacccccagtgccatcaatggcaacccatcctggcacctggcggatagcccggccgtgaatggagccactggccacagcagcagtttggatgcgcgggaggtgattcccatggcagcagtgaagcaagcgctgagagaggcaggcgatgagtttgaactgcggtaccggagagcgttcagtgatctaacatcccagcttcacataaccccagggaccgcgtatcagagctttgagcaggtagtgaatgaactctttcgggatggagtaaactggggtcgcatcgtggcctttttctcctttggcggggcactgtgcgtggaaagcgtagacaaggagatgcaggtattggtgagtcggattgcaagttggatggccacctatctgaatgaccacctagagccttggatccaggagaacggcggctgggacacttttgtggatctctacgggaacaatgcagcagccgagagccggaaaggccaggagcgcttcaaccgctggttcctgacgggcatgactgtggctggtgtggttctgctgggctcactcttcagtcggaagtga

SEQ ID NO: 79 - protein, 224 aa, Homo sapiens
  BCL 2
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLA

SEQ ID NO: 80 - DNA, 720 bp, Homo sapiens
  BCL 2
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 81 - protein, 218 aa, Mus musculus
  BCL 2
MAQAGRTGYDNREIVMKYIHYKLSQRGYEWDAGDADAAPLGAAPTPGIFSFQPESNPMPAVHRDMAARTSPLRPLVATAGPALSPVPPVVHLTLRRAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLL

SEQ ID NO: 82 - DNA, 711 bp, Mus musculus
  BCL 2
atggcgcaagccgggagaacagggtatgataaccgggagatcgtgatgaagtacatacattataagctgtcacagaggggctacgagtgggatgctggagatgcggacgcggcgcccctgggggctgcccccacccctggcatcttctccttccagcctgagagcaacccaatgcccgctgtgcaccgggacatggctgccaggacgtctcctctcaggcccctcgttgccaccgctgggcctgcgctcagccctgtgccacctgtggtccatctgaccctccgccgggctggggatgacttctctcgtcgctaccgtcgtgacttcgcagagatgtccagtcagctgcacctgacgcccttcaccgcgaggggacgctttgccacggtggtggaggaactcttcagggatggggtgaactgggggaggattgtggccttctttgagttcggtggggtcatgtgtgtggagagcgtcaacagggagatgtcacccctggtggacaacatcgccctgtggatgactgagtacctgaaccggcatctgcacacctggatccaggataacggaggctgggatgcctttgtggaactatatggccccagcatgcgacctctgtttgatttctcctggctgtctctgaagaccctgctcagcctggccctggtcggggcctgcatcactctgggtgcatacctgggccacaagtga

SEQ ID NO: 83 - protein, 350 aa, Homo sapiens
  MCL1
MFGLKRNAVIGLNLYCGGAGLGAGSGGATRPGGRLLATEKEASARREIGGGEAGAVIGGSAGASPPSTLTPDSRRVARPPPIGAEVPDVTATPARLLFFAPTRRAAPLEEMEAPAADAIMSPEEELDGYEPEPLGKRPAVLPLLELVGESGNNTSTDGSLPSTPPPAEEEEDELYRQSLEIISRYLREQATGAKDTKPMGRSGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHVEDLEGGIRNVLLAFAGVAGVGAGLAYLIR

SEQ ID NO: 84 - DNA, 1053 bp, Homo sapiens
  MCL1
atgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggggaggccggcgcggtgattggcggaagcgccggcgcaagccccccgtccaccctcacgccagactcccggagggtcgcgcggccgccgcccattggcgccgaggtccccgacgtcaccgcgacccccgcgaggctgcttttcttcgcgcccacccgccgcgcggcgccgcttgaggagatggaagccccggccgctgacgccatcatgtcgcccgaagaggagctggacgggtacgagccggagcctctcgggaagcggccggctgtcctgccgctgctggagttggtcggggaatctggtaataacaccagtacggacgggtcactaccctcgacgccgccgccagcagaggaggaggaggacgagttgtaccggcagtcgctggagattatctctcggtaccttcgggagcaggccaccggcgccaaggacacaaagccaatgggcaggtctggggccaccagcaggaaggcgctggagaccttacgacgggttggggatggcgtgcagcgcaaccacgagacggccttccaaggcatgcttcggaaactggacatcaaaaacgaagacgatgtgaaatcgttgtctcgagtgatgatccatgttttcagcgacggcgtaacaaactggggcaggattgtgactctcatttcttttggtgcctttgtggctaaacacttgaagaccataaaccaagaaagctgcatcgaaccattagcagaaagtatcacagacgttctcgtaaggacaaaacgggactggctagttaaacaaagaggctgggatgggtttgtggagttcttccatgtagaggacctagaaggtggcatcaggaatgtgctgctggcttttgcaggtgttgctggagtaggagctggtttggcatatctaataagatag

SEQ ID NO: 85 - protein, 331 aa, Mus musculus
  MCL 1
MFGLRRNAVIGLNLYCGGASLGAGGGSPAGARLVAEEAKARREGGGEAALLPGARVVARPPPVGAEDPDVTASAERRLHKSPGLLAVPPEEMAASAAAAIVSPEEELDGCEPEAIGKRPAVLPLLERVSEAAKSSGADGSLPSTPPPPEEEEDDLYRQSLEIISRYLREQATGSKDSKPLGEAGAAGRRALETLRRVGDGVQRNHETAFQGMLRKLDIKNEGDVKSFSRVMVHVFKDGVTNWGRIVTLISFGAFVAKHLKSVNQESFIEPLAETITDVLVRTKRDWLVKQRGWDGFVEFFHVQDLEGGIRNVLLAFAGVAGVGAGLAYLIR

SEQ ID NO: 86 - DNA, 996 bp, Mus musculus
  MCL1
atgtttggcctgcggagaaacgcggtcatcggcttgaacctgtactgcggcggcgccagcctcggcgcgggcggcggttctccggcaggggcgcgcctggtggccgaggaggccaaggcgcggcgcgaggggggaggggaggccgccctgctgcccggcgcgcgggtggtcgcccggccgccgcccgtgggcgccgaggaccccgacgtcaccgcgtcggccgaaaggcggctgcataagtcgcccggcctcctcgccgtgccgcccgaggagatggccgcgtcggccgccgccgccatcgtgtctccggaggaggaactggacggctgcgagccggaggccatcggcaagcgcccggccgtgctgcccctcctggagcgcgtgagcgaggcggccaagagctccggggccgacggctctctgccctccacgccgccgccgcccgaggaggaagaggacgacctataccgccagtcgctggagatcatctcgcgctacttgcgggagcaggcgaccggctccaaggactcgaagcctctgggcgaggcgggcgcggcgggccggagagcgctggagaccctgcggcgcgtgggcgacggcgtgcagcgcaaccacgagacggccttccagggcatgctccggaaactggacattaaaaacgaaggcgatgttaaatctttttctcgagtaatggtccatgttttcaaagatggcgtaacaaactggggcaggattgtgactcttatttctttcggtgcctttgtggccaaacacttaaagagcgtaaaccaagaaagcttcatcgaaccattagcagaaactatcacagatgttcttgtaaggacgaaacgggactggcttgtcaaacaaagaggctgggatgggtttgtggagttcttccacgtacaggacctagaaggcggcatcagaaatgtgctgctggcttttgcgggtgttgctggagtaggggctggtctggcatatctaataagatag

SEQ ID NO: 87 - protein, 153 aa, Homo sapiens
  IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 88 - DNA, 462 bp, Homo sapiens
  IL-2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 89 - protein, 146 aa, Mus musculus
  IL-2
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDES

SEQ ID NO: 90 - DNA, 510 bp, Mus musculus
  IL-2
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 91 - protein, 261 aa, Homo sapiens
  CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 92 - DNA, 786 bp, Homo sapiens
  CD40L
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 93 - protein, 260 aa, Mus musculus
  CD40L
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 94 - DNA, 783 bp, Mus musculus
  CD40L
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 95 - protein, 151 aa, Homo sapiens
  GITR-L
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLI

SEQ ID NO: 96 - DNA, 534 bp, Homo sapiens
  GITR-L
atgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 97 - protein, 164 aa, Mus musculus
  GITR-L
MEEMPLRESSPQRAERCKKSWLLCIVALLLMLLCSLGTLIYTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGII

SEQ ID NO: 98 - DNA, 522 bp, Mus musculus
  GITR-L
atggaggaaatgcctttgagagaatcaagtcctcaaagggcagagaggtgcaagaagtcatggctcttgtgcatagtggctctgttactgatgttgctctgttctttgggtacactgatctatacttcactcaagccaactgccatcgagtcctgcatggttaagtttgaactatcatcctcaaaatggcacatgacatctcccaaacctcactgtgtgaatacgacatctgatgggaagctgaagatactgcagagtggcacatatttaatctacggccaagtgattcctgtggataagaaatacataaaagacaatgcccccttcgtagtacagatatataaaaagaatgatgtcctacaaactctaatgaatgattttcaaatcttgcctataggaggggtttatgaactgcatgctggagataacatatatctgaagttcaactctaaagaccatattcagaaaactaacacatactgggggatcatcttaatgcctgatctaccattcatctcttag

SEQ ID NO: 99 - protein, 464 aa, Homo sapiens
  CD66a
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 100 - DNA, 1395 bp, Homo sapiens
  CD66a
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagctcaggaccactccaatga

SEQ ID NO: 101 - protein, 458 aa, Mus musculus
  CD66a
MELASAHLHKGQVPWGGLLLTASLLASWSPATTAEVTIEAVPPQVAEDNNVLLLVHNLPLALGAFAWYKGNTTAIDKEIARFVPNSNMNFTGQAYSGREIIYSNGSLLFQMITMKDMGVYTLDMTDENYRRTQATVRFHVHPILLKPNITSNNSNPVEGDDSVSLTCDSYTDPDNINYLWSRNGESLSEGDRLKLSEGNRTLTLLNVTRNDTGPYVCETRNPVSVNRSDPFSLNIIYGPDTPIISPSDIYLHPGSNLNLSCHAASNPPAQYFWLINEKPHASSQELFIPNITTNNSGTYTCFVNNSVTGLSRTTVKNITVLEPVTQPFLQVTNTTVKELDSVTLTCLSNDIGANIQWLFNSQSLQLTERMTLSQNNSILRIDPIKREDAGEYQCEISNPVSVRRSNSIKLDIIFDPTQGGLSDGAIAGIVIGVVAGVALIAGLAYFLYSRKSGGSGSF

SEQ ID NO: 102 - DNA, 1377 bp, Mus musculus
  CD66a
atggagctggcctcagcacatctccacaaagggcaggttccctggggaggactactgctcacagcctcacttttagcctcctggagccctgccaccactgctgaagtcaccattgaggctgtgccgccccaggttgctgaagacaacaatgttcttctacttgttcacaatctgcccctggcgcttggagcctttgcctggtacaagggaaacactacggctatagacaaagaaattgcacgatttgtaccaaatagtaatatgaatttcacggggcaagcatacagcggcagagagataatatacagcaatggatccctgctcttccaaatgatcaccatgaaggatatgggagtctacacactagatatgacagatgaaaactatcgtcgtactcaggcgactgtgcgatttcatgtacaccccatattattaaagcccaacatcacaagcaacaactccaatcccgtggagggtgacgactccgtatcattaacctgtgactcttacactgaccctgataatataaactacctgtggagcagaaatggtgaaagcctttcagaaggtgacaggctgaagctgtctgagggcaacaggactctcactttactcaatgtcacgaggaatgacacaggaccctatgtgtgtgaaacccggaatccagtgagtgtcaaccgaagtgacccattcagcctgaacattatctatggtccggacaccccgattatatccccctcagatatttatttgcatccagggtcaaacctcaacctctcctgccatgcagcctctaacccacctgcacagtacttttggcttatcaatgagaagccccatgcatcctcccaagagctctttatccccaacatcactactaataatagcggaacctatacctgcttcgtcaataactctgtcactggcctcagtaggaccacagtcaagaacattacagtccttgagccagtgactcagcccttcctccaagtcaccaacaccacagtcaaagaactagactctgtgaccctgacctgcttgtcgaatgacattggagccaacatccagtggctcttcaatagccagagtcttcagctcacagagagaatgacactctcccagaacaacagcatcctcagaatagaccctattaagagggaagatgccggcgagtatcagtgtgaaatctcgaatccagtcagcgtcaggaggagcaactcaatcaagctggacataatatttgacccaacacaaggaggcctctcagatggcgccattgctggcatcgtgattggagttgtggctggggtggctctaatagcagggctggcatatttcctctattccaggaagtctggcggatctggctccttctga